SEQUENCE LISTING (11 sequences)

SEQ ID NO: 1 - protein, 11 aa, Human alphaherpesvirus 1
DALDDFDLDML

SEQ ID NO: 2 - DNA, 21 bp, Artificial Sequence
  Description of Artificial Sequence Synthetic primer
ctctggagcagatttccaagg

SEQ ID NO: 3 - DNA, 20 bp, Artificial Sequence
  Description of Artificial Sequence Synthetic primer
tgccgcagttcttttgaatg

SEQ ID NO: 4 - DNA, 22 bp, Artificial Sequence
  Description of Artificial Sequence Synthetic primer
gaaagtcctattcacgccttga

SEQ ID NO: 5 - DNA, 25 bp, Artificial Sequence
  Description of Artificial Sequence Synthetic primer
caaccttcttgctcacacatgtaag

SEQ ID NO: 6 - DNA, 20 bp, Artificial Sequence
  Description of Artificial Sequence Synthetic primer
gggaaaaacaggtcatcagc

SEQ ID NO: 7 - DNA, 18 bp, Artificial Sequence
  Description of Artificial Sequence Synthetic primer
atccaccaaccaaagggc

SEQ ID NO: 8 - DNA, 24 bp, Artificial Sequence
  Description of Artificial Sequence Synthetic primer
cccactggctcctcggttctctcc

SEQ ID NO: 9 - DNA, 25 bp, Artificial Sequence
  Description of Artificial Sequence Synthetic primer
gctbgaaaggtcagcgtatggcttc

SEQ ID NO: 10 - DNA, 20 bp, Artificial Sequence
  Description of Artificial Sequence Synthetic primer
caccctcaagagcctgagtc

SEQ ID NO: 11 - DNA, 20 bp, Artificial Sequence
  Description of Artificial Sequence Synthetic primer
gttcgggctgatgtaccagt

(19) United States
(12) Patent Application Publication
(10) Pub. No.: US 2021/0236465 A1
(43) Pub. Date: Aug. 5, 2021

(54) METHODS AND COMPOSITIONS FOR TREATING HETEROTOPIC OSSIFICATION

(71) Applicant: President and Fellows of Harvard College, Cambridge, MA (US)

(72) Inventor: Yingzi Yang, Cambridge, MA (US)

(21) Appl. No.: 17/124,640

(22) Filed: Dec. 17, 2020

Related U.S. Application Data
(60) Provisional application No. 62/949,534, filed on Dec. 18, 2019.

(51) Int. Cl. A61K 31/409; A61K 31/506; A61K 31/519; A61P 19/00; G01N 33/50

(52) U.S. Cl. CPC A61K 31/409; A61K 31/506; G01N 2333/4703; A61P 19/00; G01N 33/5023; A61K 31/519

(57) ABSTRACT

The present invention provides compositions and methods for inhibiting heterotopic ossification (HO) of a cell. Other embodiments of the invention include methods of treating a subject having heterotopic ossification or a subject at risk of developing HO, as well as methods for identifying a compound that inhibits heterotopic ossification (HO) of a cell.

### RELATED APPLICATIONS

This application claims the benefit of priority to U.S. Provisional Application No. 62/949,534, filed on Dec. 18, 2019, the entire contents of which are incorporated herein by reference.

### GOVERNMENT SUPPORT

This invention was made with Government support under DE025866, AR070877, HG000187, and HG200342 awarded by the National Institutes of Health. The government, therefore, has certain rights in the invention.

### BACKGROUND OF THE INVENTION

Heterotopic ossification (HO) is a pathological condition in which bone forms in nonskeletal tissues, occurs sporadically as a common complication after trauma or surgery or in several rare, but illustrative genetic disorders (Shore, E. M. & Kaplan, F. S. Nat Rev Rheumatol 6, 518-527 (2010)). HO is still an important medical challenge, as ossifications may destroy the healing effect after surgery or injury, causing pain and limiting to a large extent of range of motions and genetic HO can be life threatening. As in normal skeletal morphogenesis, HO can form through either an intramembranous or endochondral process, suggesting that multiple mechanisms are involved (Shore, E. M. & Kaplan, F. S. Nat Rev Rheumatol 6, 518-527 (2010)). The cellular defect lies in aberrant cell-fate determination of mesenchymal progenitor cells in soft tissues, resulting in inappropriate formation of chondrocytes or osteoblasts, or both.

HO is illustrated by two rare genetic disorders that are clinically characterized by extensive and progressive bone formation in soft tissues: fibrodysplasia ossificans progressiva (FOP) and progressive osseous heteroplasia (POH) (Rigaux, P., et al. Joint Bone Spine 72, 146-149 (2005); Shafritz, A. B., et al. N Engl J Med 335, 555-561 (1996); Kaplan, F. S., Hahn, G. V. & Zasloff, M. A. J Am Acad Orthop Surg 2, 288-296 (1994)). HO in FOP is governed by endochondral bone formation, in which cartilage formation precedes bone development. FOP is caused by heterozygous change of amino acid 206 from arginine to histidine (R206H) in the bone morphogenetic protein (BMP) type I receptor known as activin receptor-like kinase 2 (ALK2), or Activin A receptor, type I (ACVR1) (Xu, R., Hu, J., Zhou, X. & Yang, Y. Bone 109, 134-142 (2018); Chakkalakal, S. A. & Shore, E. M. Methods Mol Biol 1891, 247-255 (2019); Shore, E. M., et al. Nat Genet 38, 525-527 (2006)). On the other hand, HO in POH, which is resulted from heterozygous loss of function mutations in the human GNAS gene that encodes for Gas, is governed by intramembranous bone formation, in which osteoblasts differentiate directly from mesenchymal progenitors independently of chondrocytes (Eddy, M. C., et al. J Bone Miner Res 15, 2074-2083 (2000); Shore, E. M., et al. The New England journal of medicine 346, 99-106 (2002)). Clinically, POH is characterized by dermal ossification during infancy with progressive heterotopic ossification of subcutaneous and deep connective tissue, such as muscle and fascia, during childhood (Shore, E. M., et al. The New England journal of medicine 346, 99-106 (2002)). Over time, progressively expanded HO leads to ankylosis of affected joints and growth retardation of affected limbs.

Ossification in non-hereditary HO is regulated by both endochondral and intramembranous bone formation and is a common clinical complication after trauma, including fractures, total hip arthroplasty, deep burns, and central nerve system injury (McCarthy, E. F. & Sundaram, M. Skeletal Radiol 34, 609-619 (2005); Forsberg, J. A., et al. J Bone Joint Surg Am 91, 1084-1091 (2009); Neal, B., Gray, H., MacMahon, S. & Dunn, L. ANZ J Surg 72, 808-821 (2002); Potter, B. K., Burns, T. C., Lacap, A. P., Granville, R. R. & Gajewski, D. J Am Acad Orthop Surg 14, S191-197 (2006); van Kuijk, A. A., Geurts, A. C. & van Kuppevelt, H. J. Spinal Cord 40, 313-326 (2002)). HO has an incidence of more than 50% in blast related amputations, more than 10% after a fracture, multiple injuries or total hip arthroplasty (Rigaux, P., et al. Joint Bone Spine 72, 146-149 (2005); Morley, J., Marsh, S., Drakoulakis, E., Pape, H. C. & Giannoudis, P. V. Injury 36, 363-368 (2005); Forsberg, J. A. & Potter, B. K. J Surg Orthop Adv 19, 54-61 (2010); Potter, B. K., et al. J Bone Joint Surg Am 92 Suppl 2, 74-89 (2010); Ahrengart, L. Clin Orthop Relat Res, 49-58 (1991)).

Despite the common occurrences, HO is still a major unresolved medical challenge calling for more mechanistic studies to identify effective therapeutic targets. At present, the clinical therapy is limited to surgical excision usually combined with a perioperative oral prophylaxis with nonsteroidal anti-inflammatory drugs (NSAIDs) and/or irradiation for already formed HO. These therapies, however, are associated with extremely high recurrence rates and frequent complications (Shore, E. M., et al. Nat Genet 38, 525-527 (2006); Mavrogenis, A. F., Soucacos, P. N. & Papagelopoulos, P. J. Orthopedics 34, 177 (2011)). Accordingly, there remains an ongoing and unmet need for the development of novel therapeutic strategies to treat and/or prevent HO.

### SUMMARY OF THE INVENTION

The present invention is based, at least in part, on the discovery of a Yap-Shh feedback loop as a common mechanism underlying all forms of heterotopic ossification (HO) regardless of ossification mechanism. Specifically, the present invention demonstrates that in both genetic and injury-induced HO, activated Yap and the resulting Shh expression drive HO progression by forming a positive feedback loop which results in self-propagation of osteoblast differentiation of wild type cells. In mouse models of progressive osseous heteroplasia (POH), a human disease caused by null mutations in GNAS that encodes Gas, progressively expanded ectopic bone was formed by progressively recruited wild type cells. Mechanistically, the Gnas/mesenchymal cells differentiate into osteoblasts and recruit wild type cells to form bone by activating Yap transcriptional activity, which directly activates Shh expression. Secreted Shh further induces Yap activation, Shh expression and osteoblast differentiation in surrounding wild type cells. This self-propagating positive feedback loop is both necessary and sufficient for ectopic bone formation and expansion and can act independently of Gas. Yap activation in vivo in wild type subcutaneous mesenchymal progenitor cells induced HO. Furthermore, Yap and Shh activation were found not only in fibrodysplasia ossificans progressive (FOP) and Achilles tendon puncture (ATP)-induced HO mouse models, but also in human HO samples. Importantly, genetic removal of Yap or Shh or injection of pharmacological Yap inhibitor abolished HO not only in POH, but also in FOP and acquired HO mouse models.

Therefore, the present invention successfully demonstrates that the Yap-Shh positive feedback loop, which is both necessary and sufficient to drive self-propagated osteoblast differentiation, is found in both hereditary and non-hereditary HO mouse models, and represents a common cellular and molecular mechanism underlying HO initiation and expansion that can be targeted to inhibit heterotopic ossification of a cell and to treat subjects having or at risk of developing HO.

Accordingly, in one aspect, the present invention provides a method of inhibiting heterotopic ossification (HO) of a cell. The method includes contacting the cell with an agent that inhibits the expression and/or the activity of Yes-associated protein (Yap), thereby inhibiting heterotopic ossification of the cell.

In some embodiments, the activity of Yap is transcriptional activation of sonic hedgehog (Shh) expression and/or enhancing Shh protein activity, such as enhancing Yap expression.

The cell may be a mesenchymal progenitor cell, a mesenchymal stem cell, or a mesenchyme derived differentiated cell.

In some embodiments, the contacting occurs in vitro. In other embodiments, the cell is within a subject, such as a human subject.

In some embodiments, the subject has been diagnosed with heterotopic ossification (HO). The HO may be a genetic HO disorder or a traumatic HO disorder.

In some embodiments, the genetic HO disorder is Progressive Osseous Heteroplasia (POH) or Fibrodysplasia Ossificans Progressiva (FOP).

In some embodiments, the traumatic HO disorder is due to a severe burn, a trauma to a hip, a trauma to a leg, a total hip arthroplasty, a severe fracture of a femur, a severe fracture of the humerous, a surgery to repair a severe fracture of a femur, or a surgery to repair a severe fracture of the humerous.

In some embodiments, the subject has had a surgery to remove HO tissue.

In some embodiments, the subject is at risk of developing heterotopic ossification (HO).

In some embodiments, the subject at risk of developing a traumatic HO disorder has had a severe burn, a trauma to a hip, a trauma to a leg, a total hip arthroplasty, a severe fracture of a femur, a severe fracture of the humerous, a surgery to repair a severe fracture of a femur, or a surgery to repair a severe fracture of the humerous.

The agent that inhibits the expression and/or activity of Yap may be selected from the group consisting of a small molecule, an anti-Yap antibody-or antigen-binding fragment thereof, an antisense agent targeting Yap, a double stranded RNA agent targeting Yap, an RNA-guided nuclease targeting Yap, a Yap fusion protein; a Yap inhibitory peptide; an anti-Shh antibody-or antigen-binding fragment thereof, an antisense agent targeting Shh, a double stranded RNA agent targeting Shh, an RNA-guided nuclease targeting Shh, an Shh fusion protein; an Shh inhibitory peptide, and a CDK7 inhibitor.

In some embodiments, the agent is Verteporfin (VP).

In some embodiments, the CDK7 inhibitor is THZ1 or CT7001.

In some embodiments, the methods further comprise inhibiting the expression level and/or activity of hedgehog (Hg) signaling pathway.

In other embodiments, the methods further comprise inhibiting the expression level and/or activity of Shh.

In some embodiments, the methods further comprise inhibiting the expression level and/or activity of CDK7.

In one aspect, the present invention provides a method of treating a subject having heterotopic ossification (HO) or a subject at risk of developing HO. The methods include administering to the subject a therapeutically effective amount of an inhibitor of Yes-associated protein (Yap) expression and/or the activity, thereby treating the subject.

The subject having HO may have a genetic HO disorder or a traumatic HO disorder.

In some embodiments, the genetic HO disorder is Progressive Osseous Heteroplasia (POH) or Fibrodysplasia Ossificans Progressiva (FOP).

In some embodiments, the traumatic HO disorder is due to a severe burn, a trauma to a hip, a trauma to a leg, a total hip arthroplasty, a severe fracture of a femur, a severe fracture of the humerous, a surgery to repair a severe fracture of a femur, or a surgery to repair a severe fracture of the humerous.

In some embodiments, the subject has had a surgery to remove HO tissue.

The subject at risk of developing traumatic HO disorder may have had a severe burn, a trauma to a hip, a trauma to a leg, a total hip arthroplasty, a severe fracture of a femur, a severe fracture of the humerous, a surgery to repair a severe fracture of a femur, or a surgery to repair a severe fracture of the humerous.

The agent that inhibits the expression and/or activity of Yap may be selected from the group consisting of a small molecule, an anti-Yap antibody-or antigen-binding fragment thereof, an antisense agent targeting Yap, a double stranded RNA agent targeting Yap, an RNA-guided nuclease targeting Yap, a Yap fusion protein; a Yap inhibitory peptide; an anti-Shh antibody-or antigen-binding fragment thereof, an antisense agent targeting Shh, a double stranded RNA agent targeting Shh, an RNA-guided nuclease targeting Shh, an Shh fusion protein; an Shh inhibitory peptide, and a CDK7 inhibitor.

In some embodiments, the agent is Verteporfin (VP).

In some embodiments, the CDK7 inhibitor is THZ1 or CT7001.

In ane aspect, the present invention provides a method for identifying a compound that inhibits heterotopic ossification (HO) of a cell. The methods include providing a cellular indicator composition, contacting the indicator composition with a test compound, determining the effect of the compound on the expression and/or activity of YAP in the indicator composition, wherein a decrease in the expression and/or activity of Yap indicates that the test compound inhibits HO, thereby identifying a compound that inhibits HO of the cell.

In some embodiments, the indicator composition comprises a mesenchymal progenitor cell, a mesenchymal stem cell, or a mesenchyme derived differentiated cell. In some embodiments, the indicator composition comprises a cell comprising an inactive Gnas gene. In other embodiments, the indicator composition comprises a cell comprising a constitutively active caALK2 gene. In yet other embodiments, the indicator composition comprises a cell comprising an ACVR^{R206H} gene.

In some embodiments, the methods further comprise determining the effect of the test compound on the expression and/or activity of Osx, Col1a1, Opn, Runx2, Shh, Ihh, Ctgf, Cyr61, Ankdr1, Ptch1, Gli1, Hip, Taz, Tgfβ1, Tgfβ2 and Tgfβ3.

In some embodiments, the test compound is selected from the group consisting of a small molecule, an anti-Yap antibody-or antigen-binding fragment thereof, an antisense agent targeting Yap, a double stranded RNA agent targeting Yap, an RNA-guided nuclease targeting Yap, a Yap fusion protein; a Yap inhibitory peptide; an anti-Shh antibody-or antigen-binding fragment thereof, an antisense agent targeting Shh, a double stranded RNA agent targeting Shh, an RNA-guided nuclease targeting Shh, an Shh fusion protein; an Shh inhibitory peptide, and a CDK7 inhibitor.

The present invention is illustrated by the following drawings and detailed description, which do not limit the scope of the invention described in the claims.

### BRIEF DESCRIPTION THE DRAWINGS

FIGS. 1A-1E depict that heterotopic ossification (HO) was progressively increased with progressively more wild type cells recruited in the ectopic bone in POH mouse models. Specifically, FIG. 1A depicts μCT images of the tibia bone from Gnas^{f/+}; R26^{LSL-tdTMT} (Ctrl) and Gnas^{f/f}; R26^{LSL-tdTMT} (KO) mice at indicated time after subcutaneous Ad-Cre injection at 4-week-old. Arrows indicate ectopic bone. Lines indicate section plane. n=8. Scale bar, 1 mm. FIG. 1B depicts representative von Kossa staining of the ectopic bone in the subcutaneous, muscle and deep muscular regions (boxed) from mice shown in FIG. 1A. Scale bar, 100 μm. FIG. 1C are graphs depicting Osx and Col1α1 expression analyzed by QRT-PCR in ectopic bone tissue from mice shown in FIG. 1A (upper graph) and quantification of ectopic bone volume from mice shown in FIG. 1A (lower graph). (mean±SD; n=3) **p<0.01 one-way ANOVA followed by Tukey's multiple comparisons tests. FIG. 1D depicts representative immunofluorescent images of Col1-GFP and tdTomato expression in the ectopic bone sections of the Gnas^{f/+}; Col1^{GFP+}; R26^{LSL-tdTMT} and Gnas^{f/f}; Col1^{GFP+}; R26^{LSL-tdTMT} mice injected by Ad-Cre at 4-week-old and harvested at 6 weeks, 3 months and 8 months post injection. The top panel shows the merged images with lower magnification. The boxed regions were shown in higher magnification in the lower panels. Arrows: Col1-GFP⁺; tdTomato⁻ cells. DAPI stain the nucleus. Scale bar, 100 μm. FIG. 1E depicts the number of Col1-GFP⁺, tdTomato⁻ and tdTomato⁺ cells counted in the field of view (FOV, 800×600 μm, n=5) (left panel), and the ratio of Col1-GFP⁺; tdTomato⁻ cells to total Col1-GFP⁺ cells measured in the field of view (800×600 μm, n=3) (right panel). **p<0.01 one-way ANOVA followed by Tukey's multiple comparisons tests.

FIGS. 2A-2H depict that Gnas loss induces bone formation non-cell autonomously by inducing Shh expression and secretion. Specifically, FIG. 2A depicts ALP and von Kossa staining of wildtype SMPs cultured with the conditioned medium (CM) from Ad-GFP or Ad-Cre infected SMPs for 7 days and 21 days, respectively. N=3, two sets were shown. FIG. 2B depicts quantification of ALP activities in FIG. 2A (left panel), and Osx and Col1α1 expression by QRT-PCR analysis of wild type SMPs cultured with the conditioned medium shown in FIG. 2A for 7 days (right panel) (mean±SD; n=3). **p<0.01 one-way ANOVA followed by Tukey's multiple comparisons tests. FIG. 2C depicts QRT-PCR analysis of Shh, Ihh and Dhh expression in the Gnas^{f/f} SMPs 4 days after Ad-Cre or Ad-GFP infection (left panel). Shh protein, phosphorylated Creb (pCreb), total Creb levels were also detected by Western Blotting (right panel). β-actin was loading control. FIG. 2D depicts the protein levels of secreted Shh detected by Western Blotting analysis of conditioned medium from Gnas^{f/f} SMPs 4 days following Ad-Cre or Ad-GFP infection. **p<0.01 one-way ANOVA followed by Tukey's multiple comparisons tests. FIG. 2E depicts ALP staining (top left panel), quantification of ALP activity (top right panel), and qRT-PCR analysis of Shh, Hh target gene, and osteoblast marker expression (bottom panel) of wild type SMPs cultured with the indicated conditioned medium and treated with Shh monoclonal blocker (5E1; 200 ng/ml) for 7 days. N=3, **p<0.01 one-way ANOVA followed by Tukey's multiple comparisons tests. FIG. 2F depicts ALP staining (left panel) and quantification of ALP activities (right panel) of cultured SMPs 7 days after Ad-GFP or Ad-Cre infection. N=3, **p<0.01 one-way ANOVA followed by Tukey's multiple comparisons tests. FIG. 2G depicts μCT images (left panel) and quantified ectopic bone volume (right panel) of the tibia bone with the indicated genotypes 6 weeks or 3 months post subcutaneous Ad-Cre injection at 4-week-old. Arrows indicate ectopic bone. Lines indicate section direction. n=5. Scale bar, 1 mm. **p<0.01 one-way ANOVA followed by Tukey's multiple comparisons tests. FIG. 2H depicts representative immunofluorescent images of Osx and tdTomato expression in the ectopic bone section of the indicated genotypes 3 months post Ad-Cre injection. Arrows: Osx⁺; tdTomato⁻ cells. S: Skin; M: Muscle. Scale bar, 100 μm.

FIGS. 3A-3F depict that Gnas loss upregulates Shh by activating Yap activity. Specifically, FIG. 3A depicts quantified expression levels of Yap's target genes Ctgf and Cyr61 by RNA seq analysis of Gnas^{f/f} SMPs infected by Ad-GFP or Ad-Cre (left panels). Right panel: Western blotting analyses of the indicated proteins in Gnas^{f/f} SMPs infected by Ad-GFP or Ad-Cre. (mean±SD; n=3). **p<0.01 one-way ANOVA followed by Tukey's multiple comparisons tests. FIG. 3B depicts μCT images (left panel) and quantified ectopic bone volume (right panel) of the tibia bone from mice with the indicated genotypes 6 weeks or 3 months post Ad-Cre injection at 4-week-old. Arrows indicate ectopic bone. Lines indicate section direction. n=5. Scale bar, 1 mm. **p<0.01 one-way ANOVA followed by Tukey's multiple comparisons tests. FIG. 3C depicts representative immunofluorescent images of Osx and tdTomato in the ectopic bone section of mice with indicated genotypes 6 weeks post Ad-Cre injection. Arrows: Osx⁺; tdTomato⁻ cells. S: Skin; M: Muscle. Scale bar, 100 μm. FIG. 3D depicts μCT images (top and middle left panels) and quantified ectopic bone volume (top right panel) of the tibia bone from mice with the indicated genotypes and Yap inhibitor Verteporfin (VP) treatment. HO induction and treatment schedule were shown in the lower left panel. Top left panel: VP (0.2 mg/ml) was topically applied around the injection site. Middle left panel: intraperitoneal VP injection (2.5 mg/kg). Arrows indicate ectopic bone. n=5. Scale bar, 1 mm. **p<0.01 one-way ANOVA followed by Tukey's multiple comparisons tests. FIG. 3E depicts μCT images (top panel) and quantification of ectopic bone volume (bottom panel) of the tibia bone from mice with the indicated genotypes. Doxycycline water treatment started right after Ad-Cre injection. Arrows indicate ectopic bone. n=5. Scale bar, 100 μm. FIG. 3F depicts QRT-PCR analysis of RNA isolated from ectopic bone region from the mice in FIG. 3E. Expression of Yap target gene, bone markers, transcription targets of Hh pathway and Shh/Ihh were shown. (mean±SD; n=3). *p<0.5 **p<0.01 one-way ANOVA followed by Tukey's multiple comparisons tests.

FIGS. 4A-4G depicts that Yap and Shh form a positive feedback loop. Specifically, FIG. 4A depicts ALP staining (left panel) and quantification of ALP activities (right panel) of Gnas^{f/f} SMPs 7 day after Ad-GFP or Ad-Cre infection and treatment with Yap inhibitor VP (200 ng/ml) or Shh monoclonal blocker (200 ng/ml). N=3, **p<0.01 one-way ANOVA followed by Tukey's multiple comparisons tests. FIG. 4B depicts Shh protein levels detected by Western Blotting analysis of Gnas^{f/f} SMPs as treated in FIG. 4A. GAPDH was loading control. FIG. 4C depicts ALP staining (left panel) and quantification of ALP activities (right panel) of WT SMPs cultured for 7 days with the conditioned medium (CM) from the SMPs of the indicated genotypes. N=3, **p<0.01 one-way ANOVA followed by Tukey's multiple comparisons tests. FIG. 4D depicts secreted Shh protein in the conditioned medium of Gnas^{f/f} and Gnas^{f/f} Yap^{f/f} SMPs with Ad-GFP or Ad-Cre infection detected by Western Blotting. FIG. 4E depicts representative immunofluorescent images of Yap, Shh and tdTomato expression in the ectopic bone section of mice of the indicated genotypes 3 months (left panels) or 8 months (right panels) post Ad-Cre injection. Arrows: Yap⁺; tdTomato⁻ or Shh⁺; tdTomato⁻ cells. S: Skin; M: Muscle. Scale bar, 100 μm. FIG. 4F depicts representative immunofluorescent images of Yap and tdTomato expression in the ectopic bone section from mice of the indicated genotypes 6 weeks post Ad-Cre injection. Arrows: Yap⁺; tdTomato⁻. S: Skin; M: Muscle. Scale bar, 100 μm. FIG. 4G depicts representative immunofluorescent images of Shh and tdTomato expression in the ectopic bone section from mice of the indicated genotypes 6 weeks post Ad-Cre injection. Arrows: Shh⁺; tdTomato⁻ cells. S: Skin; M: Muscle. Scale bar, 100 μm.

FIGS. 5A-5I depict that Shh is a direct target of Yap transcription factor. Specifically, FIG. 5A depicts the number of ATAC-seq peaks detected in the Ad-GFP or Ad-Cre infected Gnas^{f/f} SMPs. 43491 peaks were unique in the Ad-Cre infected SMPs. FIG. 5B depicts that unbiased de novo motif discovery was performed with the 43491 unique peaks by HOMER software and the top 5 enriched transcription factor (TF) binding motifs were found in the Ad-Cre infected Gnas^{f/f} SMPs. FIG. 5C depicts KEGG pathway mapping of the genes with transcriptional start site present in the unique ATAC peaks of the Ad-Cre infected Gnas^{f/f} SMPs. FIG. 5D depicts that the JunB and Tead4 binding peaks were closely associated in Ad-Cre infected Gnas^{f/f} SMP cells. FIG. 5E depicts the co-occurrence of JunB and Tead4 binding peaks analyzed in the unique ATAC peaks of the Ad-Cre infected Gnas^{f/f} SMPs. FIG. 5F depicts ATAC seq signal intensities analyzed around the Tead4 motifs. FIG. 5G depicts ATAC peaks of TSS and enhancer regions of the Shh locus. FIG. 5H depicts CHIP-PCR analysis of Tead4 binding sites in each indicated region (boxed). (mean±SD; n=3). **p<0.01 one-way ANOVA followed by Tukey's multiple comparisons tests. FIG. 5I depicts Shh expression in Ad-GFP (control) or Ad-Cre infected Gnas^{f/f} SMP cells (bottom panel). Tead4 sites in the Shh promotor/enhancer regions were occupied by dCas9-KRAB. A schematic of Tead4 sites blocked in the Shh promoter/enhancer regions are shown in the top panel. (mean±SD; n=3). **p<0.01 one-way ANOVA followed by Tukey's multiple comparisons tests.

FIGS. 6A-6H depict that Yap and Shh are upregulated and required for HO in a FOP mouse model. Specifically, FIG. 6A depicts μCT images (left panel) and quantified ectopic bone volume (right panel) of the tibia bone from 4-week-old ACVR1^{Q207D/+} and ACVR1^{Q207D/+} ScxCre⁺ mice. Arrows indicate ectopic bone around tendon region. Lines indicate section direction. n=5. Scale bar, 100 μm. **p<0.01 one-way ANOVA followed by Tukey's multiple comparisons tests. FIG. 6B depicts QRT-PCR analysis of gene expression in ectopic bone regions in 4-week-old ACVR1^{Q207D/+} and ACVR1^{Q207D/+} ScxCre⁺ mice. Expression of Yap target gene Ctgf, Cyr61, bone marker Osx, transcription targets of Hh pathway Ptch1, Gill and Hip, Shh/Ihh and Tgfβ1-3 were shown. (mean±SD; n=3). **p<0.01 one-way ANOVA followed by Tukey's multiple comparisons tests. FIG. 6C depicts representative immunofluorescent images of Yap, Opn (left panels) and Shh, Osx (right panels) expression in the ectopic bone section from 4-week-old ACVR1^{+/+} and ACVR1^{Q207D/+} ScxCre⁺ mice. The boxed regions were shown in higher magnification in the lower panels. Arrows: Yap⁺; Opn⁻ cells (left panel); Shh⁺; Osx⁻ cells (right panel). Scale bar, 50 μm. T: Tendon. FIG. 6D depicts ALP staining (left panel) and quaitification of ALP activities (right panel) of ACVR1^{Q207D/+} SMPs with Ad-GFP or Ad-Cre infection and VP (200 ng/ml) treatment for 7 days. N=3, **p<0.01 one-way ANOVA followed by Tukey's multiple comparisons tests. FIG. 6E depicts QRT-PCR quantification of SMPs in FIG. 6D. Expression of Yap target gene Ctgf, Cyr6, Shh/Ihh, transcription targets of Hh pathway Ptch1, Gill and Hip, and osteoblast markers Col1a1, Runx2 were shown. (mean±SD; n=3). **p<0.01 one-way ANOVA followed by Tukey's multiple comparisons tests. FIG. 6F depicts representative immunofluorescent images of Yap (left panels) or Shh (right panels) in ACVR1^{Q207D/+} SMPs with Ad-GFP or Ad-Cre infection. N=3. Scale bar, 20 μm. FIG. 6G depicts μCT images (top left panel) and quantified ectopic bone volume (top right panel) of the tibia bone from mice the indicated genotypes 6 weeks after intramuscular injection of AAV-GFP or AAV-Cre with cobra venom. Arrows indicate ectopic bone. n=64. Scale bar, 100 μm. QRT-PCR analysis of osteoblast marker Col1a1, Osx, Shh/Ihh, Yap target gene Ctgf, Cyr6 and Hh signaling target Ptch1, Gill and Hip. (mean±SD; n=3) (lower panel). **p<0.01 one-way ANOVA followed by Tukey's multiple comparisons tests.

FIG. 6H depicts μCT images (top left panel) and quantified ectopic bone volume (top right panel) of the tibia bone from mice the indicated genotypes 6 weeks after intramuscular injection of AAV-GFP or AAV-Cre with cobra venom. Arrows indicate ectopic bone. n=6. Scale bar, 100 μm. QRT-PCR analysis of osteoblast marker Col1a1, Runx2, Shh/Ihh, Yap target gene Ctgf, Cyr6 and Hh signaling target Ptch1, Gill and Hip. (mean±SD; n=3) (lower panel). **p<0.01 one-way ANOVA followed by Tukey's multiple comparisons tests.

FIGS. 7A-7H depict that Yap and Shh are upregulated in and required for HO in an injury-induced HO mouse model. Specifically, FIG. 7A depicts μCT images (top left panel) and quantified ectopic bone volume (top right panel) of the WT tibia bone and Achilles tendon region 6 weeks or 12 weeks after Achilles tendon puncture (ATP) surgery. Arrows indicate ectopic bone around tendon region. n=5. Scale bar, 100 μm. Representative images of von Kossa staining of tendon section 12 weeks post ATP surgery (bottom panel). The boxed region was shown in higher magnification in the lower panel. Scale bar, 100 μm. Arrows indicate mineralization in the injured subcutaneous region in the sham sample. FIG. 7B depicts QRT-PCR analysis of WT Achilles tendon samples 6 weeks post ATP surgery. Expression of Yap target gene Ctgf, Cyr6, osteoblast marker Col1α1 and Runx2, Shh/Ihh, Hh signaling target Ptch1, Gill and Hip and Tgfβ1-3 were shown. (mean±SD; n=3). **p<0.01 one-way ANOVA followed by Tukey's multiple comparisons tests. FIG. 7C depicts 13-Gal staining of tibia-tendon of the Ptch1^{LacZ+} mice 10 days post ATP surgery. Arrows indicate ectopic bone around the tendon. n=3. FIG. 7D depicts representative immunofluorescent images of tendon marker TNMD staining, GFP and Yap in tendon sections from the CTGF^{GFP+} mice 6 weeks post ATP surgery. The boxed region was shown in higher magnification in the lower panel. Scale bar, 50 μm. FIG. 7E depicts representative immunofluorescent images of Shh and Osx expression in the ectopic bone section from WT mice 6 weeks after ATP surgery. Arrows indicate Shh⁺Osx⁻ cells. Scale bar, 20 μm. FIG. 7F depicts μCT images (top panel) and quanfitifed ectopic bone volume (bottom panel) of the tibia/tendon region from WT mice 6 weeks after ATP surgery. VP (20 ug/ml or 200 ug/ml) and THZ1 (1 mM) were topically applied 5 days per week for 5 weeks after surgery (FIG. 7F). Arrows indicate ectopic bone around tendon region. n=5. Scale bar, 100 μm. **p<0.01 one-way ANOVA followed by Tukey's multiple comparisons tests. FIG. 7G depicts μCT images (top panel) and quanfitifed ectopic bone volume (bottom panel) of the tibia/tendon region from WT mice 6 weeks after ATP surgery. VP was IP injected 2.5 mg/kg 5 days per week for 5 weeks after surgery. Arrows indicate ectopic bone around tendon region. n=5. Scale bar, 100 μm. **p<0.01 one-way ANOVA followed by Tukey's multiple comparisons tests. FIG. 7H depicts μCT images (top panel) and quantified ectopic bone volume (bottom panel) of the tibia/tendon region from mice of the indicated genotypes 6 weeks after ATP surgery, which was performed 1 day after Ad-Cre injected to the tendon area. n=5. Scale bar, 100 μm. **p<0.01 one-way ANOVA followed by Tukey's multiple comparisons tests. FIG. 7I depicts μCT images (top panel) and quantified ectopic bone volume (bottom panel) of the tibia/tendon region of WT mice 6 weeks after ATP surgery and injection of Shh monoclonal antibody (0.25 mg/kg, 5 days per week). Arrows indicate ectopic bone around tendon region. N=5 biological replicates. Scale bar, 100 μm. **p<0.01 one-way ANOVA followed by Tukey's multiple comparisons tests.

FIGS. 8A and 8B depict up regulation of YAP and SHH in human HO. Specifically, FIG. 8A depicts gene expression analysis of YAP target gene CTGF, CYR61, SHH, HE signaling target GLI1 and osteoblast marker SP7 (OSX) and OPN by QRT-PCR of 13 human para-ossification and ossification samples from 13 patients with ossification in the posterior longitudinal ligament. (mean±SD; n=13 with 3 technical replicates for each sample). *p<0.5 **p<0.01 ***p<0.001 one-way ANOVA followed by Tukey's multiple comparisons tests. FIG. 8B depicts representative immunohistochemical images of YAP and OPN expression in the ectopic bone area of human patient. Scale bar, 50 μm. Oc: Ossification.

FIG. 9A depicts representative von Kossa staining of the ectopic bone in the subcutaneous, muscle and deep muscular regions (boxed) from 4-week-old Gnas^{f/f} R26^{LSL-tdTMT} mice 6 weeks/3 months/8 months post Adenovirus Cre injection. (left panel) Scale bar, 100 μm. The boxed region was shown in higher magnification in the right panel. Representative immunofluorescent images of Osx and tdTomato expression in the ectopic bone section of 4-week-old Gnas^{f/+} R26^{SL-tdTMT} and Gnas^{f/f} R26^{LSL-tdTMT} mice 6 weeks/3 months/8 months post Adenovirus Cre injection are shown to the right of the von Lossa stained images. Arrows: Osx positive but tdTomato negative cells. Scale bar, 100 μm. FIG. 9B depicts representative immunofluorescent images of Col1a1-GFP and tdTMT in cross sections of the hindlimb with the ectopic bone and endogenous tibia (demarcated with white lines). The Gnas^{f/+}; Col1-GFP; R26^{LS-tdTMT} and Gnas^{f/f}; Col1-GFP; R26^{LSL-tdTMT} mice were sectioned 3 months after Ad-Cre injection. Boxed regions are shown with higher magnification in the right panel. Arrows: GFP⁺; tdTMT⁻ cells. DAPI stained the nucleus. Scale bar, 100 μm. FIG. 9C depicts representative immunofluorescent images of OPN, Ki67 and tdTomato expression in the ectopic bone section of 4-week-old Gnas^{f/+} R26^{LSL-tdTMT} and Gnas^{f/f} R26^{LSL-tdTMT} mice 6 weeks post Adenovirus Cre injection. Scale bar, 100 μm. Arrows: Ki67 positive cells. Scale bar, 100 μm. S: Skin; M: Muscle. FIG. 9D depicts representative immunofluorescent images of Pdgfrα, Ki67 and tdTomato expression in the ectopic bone section of 4-week-old Gnas^{f/+} R26^{LSL-tdTMT} and Gnas^{f/f} R26^{LSL-tdTMT} mice 6 weeks post Adenovirus Cre injection. Scale bar, 100 μm. Arrows: Ki67 positive cells. Scale bar, 100 μm. S: Skin; M: Muscle.

FIG. 10A depicts QRT-PCR (top panel) and Western blot analysis (bottom panel) of Shh gene expression from ectopic bone tissue of Gnas^{f/+} R26^{LSL-tdTMT} and Gnas^{f/f} R26^{LSL-tdTMT} mice 6 weeks/3 months/8 months post Ad-Cre injection. (mean±SD; n=4). **p<0.01 one-way ANOVA followed by Tukey's multiple comparisons tests. FIG. 10B depicts QRT-PCR (top panel) and Western blot analysis (bottom panel) from Gnas^{f/+} R26^{LSL-tdTMT}, Gnas^{f/f} R26^{LSL-tdTMT} and Gnas^{f/f} Shh^{f/f} R26^{LSL-tdTMT} SMPs post Ad-GFP or Ad-Cre infection. Hh signaling, Shh and bone markers were detected by QRT-PCR. (mean±SD; n=5). **p<0.01 one-way ANOVA followed by Tukey's multiple comparisons tests. Shh protein level was detected by Western blot. FIG. 10C depicts QRT-PCR analysis of ectopic bone tissue from the indicated mice 6 weeks (6W; upper panel) and 3 months (3M; lower panel) after Ad-Cre injection. Expression of Hh signaling targets, Shh/Ihh and osteoblast markers Col1a1 and Osx are shown. (mean±SD; N=3 biological replicates). **p<0.01 one-way ANOVA followed by Tukey's multiple comparisons tests. FIG. 10D depicts that Shh deletion was detected from ectopic bone tissue in the Gnas^{f/f} Shh^{f/f} mice 6 weeks after Ad-GFP or Ad-Cre injection. Top panel: Genomic PCR to show the levels of the floxed Shh DNA. Bottom panel: Shh protein was detected by Western blotting analysis.

FIGS. 11A and 11B depict RNA sequencing of RNA samples from Gnas^{f/f} SMPs infected by Ad-GFP or Ad-Cre. Gene ontology (GO) biological process (FIG. 11B) and KEGG pathway analysis (FIG. 11A) of differentially expressed genes. Upregulations in osteoblast differentiation, ossification and mineralization were enriched and prominent increase in Hippo signaling was identified (boxed). FIG. 11C depicts QRT-PCR (top panels) and Western blot analysis (bottom panel) of Yap target gene expression from ectopic bone tissue of Gnas^{f/+} R26^{LSL-tdTMT} and Gnas^{f/f} R26^{LSL-tdTMT} mice 6 weeks/3 months/8 months post Ad-Cre injection. (mean±SD; n=4). **p<0.01 one-way ANOVA followed by Tukey's multiple comparisons tests. FIG. 11D depicts representative Immunofluorescent images of Yap/Taz and tdTomato expression in the ectopic bone section of 4-week-old Gnas^{f/+} R26^{LSL-tdTMT} Gnas^{f/f} R26^{LSL-tdTMT} mice 6 weeks (6W, left panels) or 3 months (3M, right panels) post Ad-Cre injection. Arrows: Yap/Taz positive but tdTomato negative cells. S: Skin; M: Muscle. Scale bar, 100 μm. FIG. 11E depicts serum Calcium (top left panel) and Phosphorus levels (top right panel) as well as body weights (bottom panel) of indicated mice 6 weeks, 3 months, and 8 months after Ad-Cre injection. N=6. NS: no significant difference. Ctrl: Gnas^{f/f} mice, KO: Gnas^{f/f} mice with Ad-Cre injection, DKO Shh: Gnas^{f/f}; Shh^{f/f} mice with Ad-Cre injection, DKO Yap: Gnas^{f/f}; Yap^{f/f} mice with Ad-Cre injection. Bottom panel: mouse weight was measured 4 weeks to 3 months. Adenovirus was injected at 6 weeks of age. N=6 biological replicates. NS: no significant difference. FIG. 11F depicts representative μCT images (top right panel) and quantified ectopic bone volume (bottom panel) of tibia bone regions from Gnas^{f/f} mice injected with Ad-Cre and then treated with the CDK7 inhibitors, THZ1 (10 mg/kg IP injection 5 days a week) and CT7001 (10 mg/kg garage every other day), as indicated (top left panel). Arrows point to HO. N=4. NS: no significant difference. **p<0.01 one-way ANOVA followed by Tukey's multiple comparisons tests. FIG. 11G depicts ALP staining (top left panel) and quantification of ALP activities (top right panel) of Gnas^{f/f} SMPs infected by Ad-GFP or Ad-Cre and cultured with the indicated inhibitors for 7 days. Means±SD, N=3 biological replicates. qRT-PCR analyzed gene expression by the SMPs after treatment (middle and bottom panels). NS: no significant difference. **p<0.01 one-way ANOVA followed by Tukey's multiple comparisons tests. FIG. 11H depicts μCT images (bottom panel) and quantified ectopic bone volume (top panel) of tibia bone from Gnas^{f/f} mice treated with the Yap inhibitor, Verteporfin, by intraperitoneal injection (2.5 mg/kg), 6 weeks after Ad-Cre injection, 5 times per week for 6 weeks. The treatment schedule is schematically shown (top left panel). Arrows indicate ectopic bone. N=5. Scale bar, 1 mm. FIG. 11I depicts representative μCT images (top panel) of tibia bone from indicated mice with Ad-Cre injection for 6 weeks. VP (2.5 mg/kg) was IP injected to the mice 5 days per week for 5 weeks. Trabecular bone (upper images) and cortical bone (lower images) are shown. Quantified μCT data (middle and bottom panels as means±SD. N=5. Scale bar, 100 μm. NS: no significant difference. FIG. 11J depicts representative μCT images (second row panel) and quantified ectopic bone volume (top right panel) of tibia bone from Gnas^{f/f} mice treated with the Yap inhibitor, Verteporfin, by IP injection (2.5 mg/kg), starting from the day of Ad-Cre injection for 6 weeks, then harvested immediately or kept without treatment until 3 months post injection, or VP injection for the entire 3 months post-injection. Ctrl: Gnas^{f/f} mice, KO: Gnas^{f/f} mice with Ad-Cre injection. Arrows indicate ectopic bone. Right panel: quantification of ectopic bone volume. N=5. Scale bar, 1 mm. Third row panel, left: Representative μCT images of both trabecular and cortical bones of the endogenous tibia bone. Quantified μCT data are shown as means±SD on bottom and right third-row panels. The group is VP treatment 3 months from Ad-Cre injection. N=5 biological replicates. Scale bar, 1 mm. NS: no significant difference. FIG. 11K depicts ALP staining (top left panel) and quantification of ALP activities (top right panel) of Gnas^{f/f} and Gnas^{f/f} Yap^{f/f} SMPs infected by Ad-GFP/Cre. QRT-PCR analysis of bone markers, transcription targets of Hh pathway, Shh/Ihh and Yap target gene were detected in the indicated SMPs (bottom panel). (mean±SD; n=3). **p<0.01 one-way ANOVA followed by Tukey's multiple comparisons tests. FIG. 11L is a Western blot analysis of Yap and Shh from Gnas^{f/f} and Gnas^{f/f} Yap^{f/f} SMPs infected by Ad-GFP or Ad-Cre. FIG. 11M depicts QRT-PCR analysis of ectopic bone tissue from Gnas^{f/+} R26^{LSL-tdTMT} Gnas^{f/f} R26^{LSL-tdTMT} and Gnas^{f/f} Yap^{f/f} R26^{LSL-tdTMT} mice 6 weeks (top panel) and 3 months (bottom panel) post Ad-Cre injection. Transcription targets of Hh pathway, Shh/Ihh, Yap target gene and bone markers were detected. (mean±SD; n=3). **p<0.01 one-way ANOVA followed by Tukey's multiple comparisons tests. FIG. 11N depicts ALP (upper left panel) and von Kossa (bottom left panel) staining of Yap^{tg/+}; rtTA^{tg/+} SMPs infected by Ad-GFP or Ad-Cre and cultured with Dox water for 7 days and 21 days, respectively. ALP quantification was shown on the top right panel. N=3. qRT-PCR analysis of indicated genes by Yap^{tg/+}; rtTA^{tg/+} SMPs infected by Ad-GFP or Ad-Cre and cultured with Dox water for 7 days is shown in bottom right panel. (mean±SD; N=3 biological replicates). **p<0.01 one-way ANOVA followed by Tukey's multiple comparisons tests.

FIG. 12A depicts QRT-PCR analysis of gene expression in the indicated SMPs infected by Ad-GFP or Ad-Cre with VP or Shh monoclonal antibody treatment (related to FIG. 4A) (mean±SD; N=3). **p<0.01 one-way ANOVA followed by Tukey's multiple comparisons tests. FIG. 12B depicts QRT-PCR analysis of the indicated SMPs infected by Ad-GFP or Ad-Cre (related to FIG. 4C) (mean±SD; N=3 biological replicates). **p<0.01 one-way ANOVA followed by Tukey's multiple comparisons tests. FIG. 12C depicts representative immunofluorescent images of Yap/Taz (upper panel) and Shh (middle panel) from Gnas^{f/f} SMPs with control or Ad-Cre infection. VP (200 ng/ml) or Shh monoclonal blocker (200 ng/ml) were added to the culture medium for 2 days. Scale bar, 20 μm. Quantitation of number of Yap positive cells (bottom panel). N=3. **p<0.01 one-way ANOVA followed by Tukey's multiple comparisons tests. FIG. 12D depicts representative immunofluorescent images of Yap/Taz from Gnas^{f/f} and Gnas^{f/f}; Shh^{f/f} SMPs with control or Ad-Cre infection (top left panels) and quantitation of Yap positive cells (top middle panel). Scale bar, 20 μm. N=3. Yap protein level was detected by Western blot from Gnas^{f/f} and Gnas^{f/f}Shh^{f/f} SMPs with control or Ad-Cre infection (top right panel). Representative immunofluorescent images of Shh (lower left panel) and quantitation of Shh positive cells (lower middle panel,) from Gnas^{f/f} and Gnas^{f/f} Yap^{f/f} SMPs with control or Ad-Cre infection. Scale bar, 20 μm. N=3. Shh protein level was detected by Western blot from Gnas^{f/f} and Gnas^{f/f} Yap^{f/f} SMPs with control or Ad-Cre infection (lower right panels). **p<0.01 one-way ANOVA followed by Tukey's multiple comparisons tests. FIG. 12E depicts QRT-PCR analysis from wildtype SMPs treated with recombinant Shh in the culture medium for 2 days. Expression of Yap target gene, bone markers, transcription targets of Hh pathway were shown. (mean±SD; n=3). *p<0.5 **p<0.01 one-way ANOVA followed by Tukey's multiple comparisons tests. FIG. 12F depicts ALP staining (top panel) and quantification of ALP activities (bottom left panel) of WT SMPs induced by conditioned media (CM) from the wild type SMPs (secondary CM) that had been induced by the CM from the WT, Gnas^{-/-}, Gnas^{-/-}; Yap^{-/-}, Gnas^{-/-}; Shh^{-/-}, and Gnas^{-/-} cells with the Shh mAb. N=3. **p<0.01 one-way ANOVA followed by Tukey's multiple comparisons tests. QRT-PCR analysis of the indicated SMPs infected by Ad-GFP or Ad-Cre with Shh monoclonal antibody treatment (bottom right panel). (mean±SD; N=3 biological replicates). **p<0.01 one-way ANOVA followed by Tukey's multiple comparisons tests. FIG. 12G depicts schematics of self-propagation of osteoblast differentiation by a positive feedback loop of Yap and Shh. The Hh signaling and Yap activities are activated in the initial Gnas^{-/-} SMPs (primary induction). These cells differentiate into osteoblasts, but also express Shh directly controlled by Yap activation. Secreted Shh activate Hh signaling and Yap in surrounding wild type cells (1⁰ amplification), which converts these cells to osteoblasts and Shh secreting cells (2⁰ amplification).

FIG. 13A depicts the Runx2 and Tead4 binding peaks detected in Gnas^{-/-} SMPs by ATAC-seq analysis. FIG. 13B depicts the co-occurrence of Runx2 and Tead4 binding peaks analyzed in the unique ATAC peaks of the Gnas^{-/-} SMPs.

FIG. 14A depicts μCT images of the tibia bone from 4-week-old ACVR1^{Q207D/+} ACVR1^{Q207D/+} ScxCre⁺ and ACVR1^{Q207D/+} Yap^{f/+} ScxCre⁺ mice (left panels). Arrows indicate ectopic bone. n=6. Scale bar, 1 mm. QRT-PCR analysis of ectopic bone tendon from 4-week-old ACVR1^{Q207D/+} ACVR1^{Q207D/+} ScxCre⁺ and ACVR1^{Q207D/+} Yap^{f/+} ScxCre⁺ mice was done. Expression of Yap target gene, bone markers, transcription targets of Hh pathway and Shh/Ihh were shown (right panel). (mean±SD; n=3). **p<0.01 one-way ANOVA followed by Tukey's multiple comparisons tests. FIG. 14B depicts representative μCT images (top left panel) and quantified ectopic bone volume (top left panel) of tibia bone regions from the 3-month-old ScxCre; ACVR1^{F1ExR206H/+} mice. Arrows indicate ectopic bone. N=3. Scale bar, 100 μm. Middle and bottom panels depict qRT-PCR analysis of gene expression in ectopic bone formed in the Achilles tendon of indicated mice at 1.5 and 3 months old. (mean±SD; N=3 biological replicates). **p<0.01 one-way ANOVA followed by Tukey's multiple comparisons tests. FIG. 14C depicts μCT images (top panel) and quantified ectopic bone volume (bottom panel) of hind limbs from ScxCre; ACVR1^{FlExR206H/+} mice that were injected with VP (4 mg/kg) 5 days a week for 8 weeks. Arrows indicate HO areas. The HO volume was quantified in Vehicle and VP injection groups. (mean±SD; n=5). **p<0.01 one-way ANOVA followed by Tukey's multiple comparisons tests.

FIG. 15 depicts μCT images of the tibia bone from 4-week-old wildtype mice with ATP surgery for 6 weeks (top panels). VP (2.5 mg/kg) was IP injected to the mice 5 days per week for 5 weeks. Trabecular bone (upper portion of top panel) and cortical bone (lower portion of top panel) were shown. Quantified analysis of μCT data are shown in the lower panels. Data are shown as means±SD. n=5. Scale bar, 100 μm. NS: no significant difference.

### DETAILED DESCRIPTION OF THE INVENTION

The present invention is based, at least in part, on the discovery of a Yap-Shh feedback loop as a common mechanism underlying all forms of heterotopic ossification (HO) regardless of ossification mechanism. Specifically, the present invention demonstrates that in both genetic and injury-induced HO, activated Yap and the resulting ectopic Shh expression drive HO progression by forming a positive feedback loop that results in self-propagation of osteoblast differentiation of wild type cells. In mouse models of progressive osseous heteroplasia (POH), a human disease caused by null mutations in GNAS that encodes Gas, progressively expanded ectopic bone was formed by progressively recruited wild type cells. Mechanistically, the Gnas/mesenchymal cells differentiate into osteoblasts and recruit wild type cells to form bone by activating Yap transcriptional activity, which directly activates Shh expression. Secreted Shh further induces Yap activation, Shh expression and osteoblast differentiation in surrounding wild type cells. This self-propagating positive feedback loop is both necessary and sufficient for ectopic bone formation and expansion and can act independently of Gas. Yap activation in vivo in wild type subcutaneous mesenchymal progenitor cells induced HO. Furthermore, Yap and Shh activation were found not only in fibrodysplasia ossificans progressive (FOP) and Achilles tendon puncture (ATP)-induced HO mouse models, but also in human HO samples. Importantly, genetic removal of Yap or Shh or injection of a pharmacological Yap inhibitor abolished HO not only in POH, but also in FOP and acquired HO mouse models. Therefore, the present invention successfully demonstrates that the Yap-Shh positive feedback loop, which is both necessary and sufficient to drive self-propagated osteoblast differentiation, is found in both hereditary and non-hereditary HO mouse models, and represents a common cellular and molecular mechanism underlying HO initiation and expansion.

Accordingly, the present invention provides methods and compositions for inhibiting heterotopic ossification (HO) of a cell by contacting the cell with an agent that inhibits the expression and/or the activity of Yes-associated protein (Yap). The present invention also features, in other embodiments, methods and compositions for treating a subject having heterotopic ossification (HO) or a subject at risk of developing HO by administering to the subject a therapeutically effective amount of an inhibitor of Yap expression and/or the activity. In further embodiments, the invention includes methods for identifying a compound that inhibits heterotopic ossification (HO) of a cell.

### I. Definitions

In order that the present invention may be more readily understood, certain terms are first defined. In addition, it should be noted that whenever a value or range of values of a parameter are recited, it is intended that values and ranges intermediate to the recited values are also part of this invention.

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

The articles “a” and “an” are used herein to refer to one or to more than one (i.e., to at least one) of the grammatical object of the article. By way of example, “an element” means one element or more than one element.

The term “comprising” or “comprises” is used herein in reference to compositions, methods, and respective component(s) thereof, that are essential to the invention, yet open to the inclusion of unspecified elements, whether essential or not.

The term “consisting of” refers to compositions, methods, and respective components thereof as described herein, which are exclusive of any element not recited in that description of the embodiment.

As used herein, the term “Yap” or “Yes-associated protein”, also known as Yap1, Yap2, Yki, Cob 1 or Yap65, refers to a protein that acts as a transcriptional factor in the Hippo signaling pathway that critically regulates cells proliferation, differentiation and survival in development and tumorigenesis (Pan, D. Dev Cell 19, 491-505, (2010); Barron, D. A. & Kagey, J. D. Clin Transl Med 3, 25 (2014); Mo, J. S., Park, H. W. & Guan, K. L. EMBO Rep 15, 642-656 (2014)).

In one embodiment, Yap is a human Yap (Gene ID: 10413).

In one embodiment, the sequence of a human Yap mRNA (variant 1) is set forth as GENBANK Accession No. NM_001130145.3, GI:1519243969. In this embodiment, the sequence of a human Yap polypeptide sequence (isoform 1) is set forth in GENBANK Accession No. NP_001123617.1, GI:194306653.

In one embodiment, the sequence of a human Yap mRNA (variant 2) is set forth as GENBANK Accession No. NM_006106.5, GI:1676439961. In this embodiment, the sequence of a human Yap polypeptide sequence (isoform 2) is set forth in GENBANK Accession No. NP_006097.2, GI:303523511.

In one embodiment, the sequence of a human Yap mRNA (variant 3) is set forth as GENBANK Accession No. NM_001195044.2, GI:1676317993. In this embodiment, the sequence of a human Yap polypeptide sequence (isoform 3) is set forth in GENBANK Accession No. NP_001181973.1, GI:303523610.

In one embodiment, the sequence of a human Yap mRNA (variant 4) is set forth as GENBANK Accession No. NM_001195045.2, GI:1676439708. In this embodiment, the sequence of a human Yap polypeptide sequence (isoform 4) is set forth in GENBANK Accession No. NP_001181974.1, GI:303523627.

In one embodiment, the sequence of a human Yap mRNA (variant 5) is set forth as GENBANK Accession No. NM_001282098.2, GI:1675158166. In this embodiment, the sequence of a human Yap polypeptide sequence (isoform 5) is set forth in GENBANK Accession No. NP_001269027.1, GI:530788246.

In one embodiment, the sequence of a human Yap mRNA (variant 6) is set forth as GENBANK Accession No. NM_001282097.2 GI: 1676325088. In this embodiment, the sequence of a human Yap polypeptide sequence (isoform 6) is set forth in GENBANK Accession No. NP_001269026.1 GI: 530788244.

In one embodiment, the sequence of a human Yap mRNA (variant 7) is set forth as GENBANK Accession No. NM_001282099.1 GI: 530788247. In this embodiment, the sequence of a human Yap polypeptide sequence (isoform 7) is set forth in GENBANK Accession No. NP_001269028.1 GI: 530788248.

In one embodiment, the sequence of a human Yap mRNA (variant 8) is set forth as GENBANK Accession No. NM_001282100.1 GI: 530788249. In this embodiment, the sequence of a human Yap polypeptide sequence (isoform 8) is set forth in GENBANK Accession No. NP_001269029.1 GI: 530788250.

In one embodiment, the sequence of a human Yap mRNA (variant 9) is set forth as GENBANK Accession No. NM_001282101.1 GI: 530788251. In this embodiment, the sequence of a human Yap polypeptide sequence (isoform 9) is set forth in GENBANK Accession No. NP_001269030.1 GI: 530788252.

In one embodiment, Yap is a mouse Yap (Gene ID: 22601).

In one embodiment, the sequence of a mouse (Mus musculus) Yap mRNA (variant 1) is set forth as GENBANK Accession No. NM_001171147.1 GI: 283945492. In this embodiment, the sequence of a mouse (Mus musculus) Yap polypeptide sequence (isoform 1) is set forth in GENBANK Accession No. XP 006509915.1 GI: 568958510.

It should be noted that throughout, molecule names, e.g., Yap, include the gene, mRNA and protein, unless otherwise specified. Thus, the term “Yap” when used in reference to the molecule includes Yap protein, Yap mRNA and the Yap gene.

As used herein, the term “Yap expression”, refers to transcription of the gene encoding Yap, i.e., Yap mRNA or translation of Yap mRNA, i.e., Yap protein. Thus, Yap expression, as used herein, refers to the presence of Yap in either protein or nucleic acid form, unless otherwise specified.

As used herein, the term “Yap activity”, refers to the transcriptional activation of target genes, e.g., activating, e.g., directly activating, the transcription of sonic hedgehog (Shh), increasing Shh expression, and/or enhancing Shh protein activity.

As used herein, the term “Sonic hedgehog (Shh),” also known as, SMMCI, TPTPS, HHG-1, HLP3, HPE3 and TPT, refers to a secreted ligand that activates Hedgehog signaling. Shh was only found previously to be required for early embryonic growth and patterning, but not bone formation.

In one embodiment, Shh is a human Shh (Gene ID: 6469).

In one embodiment, the sequence of a human Shh mRNA (variant 1) is set forth as GENBANK Accession No. NM_000193.4 GI: 1519245148. In this embodiment, the sequence of a human Shh polypeptide sequence (isoform 1) is set forth in GENBANK Accession No. NP_000184.1, GI:4506939.

In one embodiment, the sequence of a human Shh mRNA (variant 2) is set forth as GENBANK Accession No. NM_001310462.2 GI: 1676344109. In this embodiment, the sequence of a human Shh polypeptide sequence (isoform 2) is set forth in GENBANK Accession No. NP_001297391.1 GI: 881717124.

In one embodiment, Shh is a mouse Shh (Gene ID: 20423).

In one embodiment, the sequence of a mouse (Mus musculus) Shh mRNA is set forth as GENBANK Accession No. NM_009170.3 GI: 161484664. In this embodiment, the sequence of a mouse (Mus musculus) Shh polypeptide sequence is set forth in GENBANK Accession No. XP_NP_033196.1 GI: 21617861.

As used herein, the term “an agent inhibits the expression and/or activity of Yap” or “an Yap inhibitor” refers to an agent which directly or indirectly interferes with the expression and/or activity of Yap. In one embodiment, the invention includes a method of contacting a cell with an agent that inhibits the expression and/or activity of Yap, such that Yap expression and/activity is reduced in the cell, and heterotopic ossification is inhibited. In another embodiment, the invention includes a method for treating a subject having heterotopic ossification (HO) or a subject at risk of developing HO by administering to the subject a therapeutically effective amount of an inhibitor of Yap expression and/or the activity.

In one embodiment, the inhibitor of Yap acts directly on Yap, e.g., an antibody or a small molecule, which binds to Yap and inhibits its function. In another embodiment, the inhibitor of Yap acts indirectly, e.g., through another molecule which interacts with Yap, e.g., Sonic Hedgehog (Shh), or CDK7, thereby decreasing Yap expression and/or activity.

The phrase “contacting a cell with an agent,” such as an agent that inhibits the expression and/or activity of Yap, as used herein, includes contacting a cell by any possible means. Contacting a cell with an agent includes contacting a cell in vitro with the agent or contacting a cell in vivo with the agent. The contacting may be done directly or indirectly. Thus, for example, the agent may be put into physical contact with the cell by the individual performing the method, or alternatively, the agent may be put into a situation that will permit or cause it to subsequently come into contact with the cell.

As used herein, a “subject” is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird that expresses the target gene, either endogenously or heterologously. In one embodiment, the subject is a mammal. In another embodiment, the subject is a human, such as a human being treated or assessed for a heterotopic ossification disease or disorder that would benefit from reduction in Yap expression and/or activity; a human at risk for developing a heterotopic ossification disease or disorder that would benefit from reduction in Yap expression and/or activity; a human having a heterotopic ossification disease or disorder that would benefit from reduction in Yap expression and/or activity; or human being treated for a heterotopic ossification disease or disorder that would benefit from reduction in Yap expression and/or activity, for example, human being who has previously had a surgery to remove heterotopic ossification tissue; as described herein. In some embodiments, the subject is a female human. In other embodiments, the subject is a male human. In one embodiment, the subject is an adult subject. In another embodiment, the subject is a pediatric subject.

In some embodiments, the subject having HO has a genetic HO disorder. In some embodiments, the genetic HO disorder is Progressive Osseous Heteroplasia (POH). In other embodiments, the genetic HO disorder is Fibrodysplasia Ossificans Progressiva (FOP).

In some embodiments, the subject having HO has a traumatic HO disorder. In some embodiments, the traumatic HO disorder is due to a traumatic brain injury, a traumatic spinal cord injury, a severe burna trauma to a hip, a trauma to a leg, a total hip arthroplasty, a severe fracture of a femur, a severe fracture of the humerous, a surgery to repair a severe fracture of a femur, a surgery to repair a severe fracture of the humerous, or a lower motor neuron disorder.

As used herein, the terms “treating” or “treatment” refer to a beneficial or desired result, such as reducing at least one sign or symptom of a heterotopic ossification disease or disorder in a subject. Treatment also includes a reduction of one or more sign or symptoms associated with Yap expression; diminishing the extent of Yap activation or stabilization; amelioration or palliation of Yap activation or stabilization. “Treatment” can also mean prolonging survival as compared to expected survival in the absence of treatment.

The term “lower”, “decreased” or “reduced” in the context of the level of Yap in a subject or a disease marker or symptom refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. “Lower”, “decreased” or “reduced” in the context of the level of Yap in a subject is preferably down to a level accepted as within the range of normal for an individual without such disorder. In certain embodiments, the expression of the target is normalized, i.e., decreased towards or to a level accepted as within the range of normal for an individual without such disorder, e.g., normalization of body weight, blood pressure, or a serum lipid level. As used here, “lower”, “decreased” or “reduced” in a subject can refer to lowering of gene expression or protein production in a cell in a subject does not require lowering of expression in all cells or tissues of a subject.

### II. Methods of the Invention

The methods and compositions of the present invention are useful for inhibiting heterotopic ossification (HO), as well as treating a subject having heterotopic ossification or a subject at risk of developing HO. The inventors successfully identified a Yap-Shh feedback loop as a common mechanism underlying all forms of heterotopic ossification (HO) regardless of ossification mechanisms. As shown in the Examples presented herein, genetic removal of Yap or Shh or injection of pharmacological Yap inhibitor abolished HO not only in a genetic HO disorder, such as progressive osseous heteroplasia (POH) and fibrodysplasia ossificans progressive (FOP), but also abolished HO in traumatic HO disorders, e.g., Achilles tendon puncture (ATP)-induced HO mouse models, suggesting that Yap and Shh are indeed promising therapeutic targets for HO treatment.

Accordingly, the present invention, in one aspect, provides methods for inhibiting heterotopic ossification of a cell. The methods comprise contacting the cell with an agent that inhibits the expression and/or the activity of Yes-associated protein (Yap), thereby inhibiting heterotopic ossification of the cell.

Heterotopic ossification (HO) is a pathological condition in which bone forms in nonskeletal tissues, occurs sporadically as a common complication after trauma or surgery or in several rare, but illustrative genetic disorders (Shore, E. M. & Kaplan, F. S. Nat Rev Rheumatol 6, 518-527 (2010)). Specifically, HO is illustrated by two rare genetic disorders that are clinically characterized by extensive and progressive bone formation in soft tissues: fibrodysplasia ossificans progressiva (FOP) and progressive osseous heteroplasia (POH) (Rigaux, P., et al. Joint Bone Spine 72, 146-149 (2005); Shafritz, A. B., et al. N Engl J Med 335, 555-561 (1996); Kaplan, F. S., Hahn, G. V. & Zasloff, M. A. J Am Acad Orthop Surg 2, 288-296 (1994)). HO in FOP is governed by endochondral bone formation, in which cartilage formation precedes bone development. FOP is caused by heterozygous change of amino acid 206 from arginine to histidine (R206H) in the bone morphogenetic protein (BMP) type I receptor known as activin receptor-like kinase 2 (ALK2), or Activin A receptor, type I (ACVR1) (Xu, R., Hu, J., Zhou, X. & Yang, Y. Bone 109, 134-142 (2018); Chakkalakal, S. A. & Shore, E. M. Methods Mol Biol 1891, 247-255 (2019); Shore, E. M., et al. Nat Genet 38, 525-527 (2006)). On the other hand, HO in POH, which is resulted from heterozygous loss of function mutations in the human GNAS gene that encodes for Gas, is governed by intramembranous bone formation, in which osteoblasts differentiate directly from mesenchymal progenitors independently of chondrocytes (Eddy, M. C., et al. J Bone Miner Res 15, 2074-2083 (2000); Shore, E. M., et al. The New England journal of medicine 346, 99-106 (2002)). Clinically, POH is characterized by dermal ossification during infancy with progressive heterotopic ossification of subcutaneous and deep connective tissue, such as muscle and fascia, during childhood (Shore, E. M., et al. The New England journal of medicine 346, 99-106 (2002)). Over time, progressively expanded HO leads to ankylosis of affected joints and growth retardation of affected limbs.

Ossification in non-hereditary HO is regulated by both endochondral and intramembranous bone formation and is a common clinical complication after trauma, including fractures, total hip arthroplasty, deep burns, and central nerve system injury (McCarthy, E. F. & Sundaram, M. Skeletal Radiol 34, 609-619 (2005); Forsberg, J. A., et al. J Bone Joint Surg Am 91, 1084-1091 (2009); Neal, B., Gray, H., MacMahon, S. & Dunn, L. ANZ J Surg 72, 808-821 (2002); Potter, B. K., Burns, T. C., Lacap, A. P., Granville, R. R. & Gajewski, D. J Am Acad Orthop Surg 14, S191-197 (2006); van Kuijk, A. A., Geurts, A. C. & van Kuppevelt, H. J. Spinal Cord 40, 313-326 (2002)).

The methods of the present invention are suitable for inhibiting heterotopic ossification in both genetic HO disorders, e.g., Progressive Osseous Heteroplasia (POH) or Fibrodysplasia Ossificans Progressiva (FOP), and traumatic HO disorders, e.g., a traumatic HO disorder due to a traumatic brain injury, a traumatic spinal cord injury, a severe burna trauma to a hip, a trauma to a leg, a total hip arthroplasty, a severe fracture of a femur, a severe fracture of the humerous, a surgery to repair a severe fracture of a femur, a surgery to repair a severe fracture of the humerous, or a lower motor neuron disorder.

In some embodiments, the methods are also suitable for subjects who have had a surgery to remove HO tissue previously, or who are at risk of developing a HO disorder, e.g., both genetic HO disorders and traumatic HO disorders.

The methods of the present invention comprise contacting a cell with agents that inhibit the expression and/or activity of Yap in order to inhibit heterotopic ossification (HO) in a cell. Contacting a cell with an agent includes contacting a cell in vitro with the agent or contacting a cell in vivo with the agent. The contacting may be done directly or indirectly. Thus, for example, the agent may be put into physical contact with the cell by the individual performing the method, or alternatively, the agent may be put into a situation that will permit or cause it to subsequently come into contact with the cell.

Contacting a cell in vitro may be done, for example, by incubating the cell with the agent. Contacting a cell in vivo may be done, for example, by injecting the agent into or near the tissue where the cell is located, or by injecting the agent into another area, e.g., the peritoneal cavity, the bloodstream, or the subcutaneous space, such that the agent will subsequently reach the tissue where the cell to be contacted is located. For example, the agent may contain or be coupled to a ligand that directs the agent to a site of interest. Combinations of in vitro and in vivo methods of contacting are also possible. For example, a cell may also be contacted in vitro with an agent and subsequently transplanted into a subject.

In certain embodiments, contacting a cell with an agent includes “introducing” or “delivering the agent into the cell” by facilitating or effecting uptake or absorption into the cell. Absorption or uptake of an agent can occur through unaided diffusion or active cellular processes, or by auxiliary agents or devices. Introducing an agent into a cell may be in vitro or in vivo. For example, for in vivo introduction, agent can be injected into a tissue site or administered systemically. The agent can also be injected intraperitoneally. Alternatively, the agent can be applied topically to the target site, e.g., the bone tissue. In vitro introduction into a cell includes methods known in the art such as electroporation and lipofection. Further approaches are described herein below or are known in the art

In some embodiments, the cells are subcutaneous mesenchymal progenitor cells, mesenchyme derived differentiated cells, or heterotopic ossified cells.

By contacting cells with agents that inhibit the expression and/or activity of Yap, heterotopic ossification in the cells are reduced. The agents may functions at a level of transcription and mRNA stability, or they may act at a level of translation, protein stability/degradation, protein modification, and protein binding.

In some embodiments, the activity of Yap comprises the transcriptional activation of sonic hedgehog (Shh) expression and/or enhancing Shh protein activity. In other embodiments, the Shh protein activity is to potentiate Yap expression, thus forming a positive feedback loop.

In another aspect, the present invention provides methods for treating a subject having heterotopic ossification (HO) or a subject at risk of developing HO. The methods comprise administering to the subject a therapeutically effective amount of an inhibitor of Yes-associated protein (Yap) expression and/or the activity, thereby treating the subject.

In some embodiments, the subject has a genetic HO disorder, e.g., Progressive Osseous Heteroplasia (POH) or Fibrodysplasia Ossificans Progressiva (FOP). In other embodiments, the subject has a traumatic HO disorder, e.g., a traumatic HO disorder due to a traumatic brain injury, a traumatic spinal cord injury, a severe burna trauma to a hip, a trauma to a leg, a total hip arthroplasty, a severe fracture of a femur, a severe fracture of the humerous, a surgery to repair a severe fracture of a femur, a surgery to repair a severe fracture of the humerous, or a lower motor neuron disorder.

In some embodiments, the methods are also suitable for subjects who have had a surgery to remove HO tissue previously, or who are at risk of developing a HO disorder, e.g., both genetic HO disorders and traumatic HO disorders.

In another aspect, the presentation provides a method for identifying a compound that inhibits heterotopic ossification (HO) of a cell. The method comprises providing a cellular indicator composition, contacting the indicator composition with a test compound, determining the effect of the compound on the expression and or activity of YAP in the indicator composition, wherein a decrease in the expression and/or activity of Yap indicates that the test compound inhibits HO, thereby identifying a compound that inhibits HO of the cell.

In some embodiments, the indicator composition comprises a mesenchymal progenitor cell, a mesenchymal stem cell, a mesenchyme derived differentiated cell or a heterotopic ossified cell. The cells suitable for the methods of the invention may comprise an inactive Gnas gene, or a constitutively active caALK2 gene. Alternatively, the cell may comprise an ACVR^{R206H} gene.

In addition to have an effect on the expression and/or activity of Yap in the indicator composition, the test compound may also have an effect on the expression and/or activity of several other markers, e.g., Osx, Col1α1, Opn, Runx2, Shh, Ihh, Ctgf, Cyr61, Ankdr1, Ptch1, Gli1, Hip, Taz, Tgfβ 1, Tgfβ2 and Tgfβ3. Accordingly, the methods of the present invention further comprise determining the expression and/or activity of these markers in order to identify a compound that inhibits HO of a cell.

Pharmaceutical compositions described herein are suitable for administration in human or non-human subjects. Accordingly, the agent that inhibits the expression and/or activity of Yap, e.g., a small molecule inhibitor of Yap, and/or an anti-Yap antibody or antigen-binding portion thereof, described herein are useful as medicament for administering to a subject who is likely to benefit from reduced Yap expression and/or activity. In some embodiments, suitable subjects include healthy individuals who may nonetheless benefit from reduced Yap expression and/or activity. In some embodiments, suitable subjects have an existing heterotopic ossification condition.

In some embodiments, the subject having HO has a genetic HO disorder. In some embodiments, the genetic HO disorder is Progressive Osseous Heteroplasia (POH). In other embodiments, the genetic HO disorder is Fibrodysplasia Ossificans Progressiva (FOP).

In some embodiments, the subject having HO has a traumatic HO disorder. In some embodiments, the traumatic HO disorder is due to a traumatic brain injury, a traumatic spinal cord injury, a severe burna trauma to a hip, a trauma to a leg, a total hip arthroplasty, a severe fracture of a femur, a severe fracture of the humerous, a surgery to repair a severe fracture of a femur, a surgery to repair a severe fracture of the humerous, or a lower motor neuron disorder. In some embodiments, suitable subjects are at risk of developing heterotopic ossification disorders. In some embodiments, suitable subjects are those who have previously had a surgery to remove heterotopic ossification tissues. In some embodiments, suitable subjects are those on a therapy comprising another therapeutic agent, e.g., a perioperative oral prophylaxis with nonsteroidal anti-inflammatory drugs (NSAIDs) and/or irradiation for already formed HO, to treat heterotopic ossification, however, these therapies are associated with adverse effects or high recurrence rates.

In some embodiments, such medicament is suitable for administration in a pediatric population, an adult population, and/or an elderly population.

The pediatric population in need for the agents inhibiting the expression and/or activity of Yap, e.g., small molecule inhibitors of Yap, or anti-Yap antibodies and antigen-binding portions thereof, described herein may range between 0 and 6 months of age, between 0 and 12 months of age, between 0 and 18 months of age, between 0 and 24 months of age, between 0 and 36 months of age, between 0 and 72 months of age, between 6 and 36 months of age, between 6 and 36 months of age, between 6 and 72 months of age, between 12 and 36 months of age, between 12 and 72 months of age. In some embodiments, the pediatric population suitable for receiving the agents inhibiting the expression and/or activity of Yap, e.g., small molecule inhibitors of Yap, or anti-Yap antibodies and antigen-binding portions thereof, described herein who is likely to benefit from such treatment may range between 0 and 6 years of age, between 0 and 12 years of age, between 3 and 12 years of age, between 0 and 17 years of age. In some embodiments, the population has an age of at least 5 years, e.g., 6, 7, 8, 9, 10, 11, 12, 13, 14, 15, 16, or 17 years. In some embodiments, the pediatric population may be aged below 18 years old. In some embodiments, the pediatric population may be (a) at least 5 years of age and (b) below 18 years of age.

The adult population in need for the agents inhibiting the expression and/or activity of Yap, e.g., small molecule inhibitors of Yap, or anti-Yap antibodies and antigen-binding portions thereof, described herein may have an age of at least 18 years, e.g., at least 19, 20, 25, 30, 35, 40, 45, 50, 55, 60 or 65 years. In some embodiments, the adult population may be below 65 years of age. In some embodiments, the adult population may of (a) at least 18 years of age and (b) below 65 years of age.

The elderly population in need for the agents inhibiting the expression and/or activity of Yap, e.g., small molecule inhibitors of Yap, or anti-Yap antibodies and antigen-binding portions thereof, described herein may have an age of 65 years or older (i.e., ≥65 years old), e.g., at least 70, 75 or 80 years.

A human subject who is likely to benefit from the treatment may be a human patient having, at risk of developing, or suspected of having heterotopic ossification, such as those described below. A subject having heterotopic ossification can be identified by routine medical examination, e.g., laboratory tests, organ functional tests, CT scans, or ultrasounds. A subject suspected of having any of such disease/disorder might show one or more symptoms of the disease/disorder. A subject at risk for the disease/disorder can be a subject having one or more of the risk factors for that disease/disorder.

A control subject, as described herein, is a subject who provides an appropriate reference for evaluating the effects of a particular treatment or intervention of a test subject or subject. Control subjects can be of similar age, race, gender, weight, height, and/or other features, or any combination thereof, to the test subjects.

In some embodiments, the agents that inhibit the expression and/or activity of Yap, e.g., small molecule inhibitors of Yap, or anti-Yap antibodies and antigen-binding portions thereof, described herein are administered to a subject in need of the treatment at an amount sufficient to inhibit heterotopic ossification by at least 10% (e.g., 20% 30%, 40%, 50%, 60%, 70%, 80%, 90% or greater) in vivo. In other embodiments, the agents that inhibit the expression and/or activity of Yap, e.g., small molecule inhibitors of Yap, or anti-Yap antibodies and antigen-binding portions thereof, are administered in an amount effective in reducing heterotopic ossification by at least 10% (e.g., 20%, 30%, 40%, 50%, 60%, 70%, 80%, 90% or greater) in vitro.

In some embodiments, the agents that inhibit the expression and/or activity of Yap, e.g., small molecule inhibitors of Yap, or anti-Yap antibodies and antigen-binding portions thereof, are administered to a subject who will benefit from decreased expression and/or activity of Yap. In some embodiments, the agents that inhibit the expression and/or activity of Yap, e.g., small molecule inhibitors of Yap, or anti-Yap antibodies and antigen-binding portions thereof, are administered to a subject who has heterotopic ossification. In some embodiments, the agents that inhibit the expression and/or activity of Yap, e.g., small molecule inhibitors of Yap, or anti-Yap antibodies and antigen-binding portions thereof, are administered to a subject who has a risk of developing heterotopic ossification disorders. In some embodiments, the agents that inhibit the expression and/or activity of Yap, e.g., small molecule inhibitors of Yap, or anti-Yap antibodies and antigen-binding portions thereof, are administered to a subject who has a genetic heterotopic ossification disorder. In one embodiment, the genetic heterotopic ossification disorder is progressive osseous heteroplasia (POH). In another embodiment, the genetic heterotopic ossification disorder is fibrodysplasia ossificans progressive (FOP). In some embodiments, the agents that inhibit the expression and/or activity of Yap, e.g., small molecule inhibitors of Yap, or anti-Yap antibodies and antigen-binding portions thereof, are administered to a subject who has a trauma-induced heterotopic ossification disorder. In some embodiments, the traumatic heterotopic ossification disorder is due to a traumatic brain injury, a traumatic spinal cord injury, a severe burna trauma to a hip, a trauma to a leg, a total hip arthroplasty, a severe fracture of a femur, a severe fracture of the humerous, a surgery to repair a severe fracture of a femur, a surgery to repair a severe fracture of the humerous, or a lower motor neuron disorder. In some embodiments, the agents that inhibit the expression and/or activity of Yap, e.g., small molecule inhibitors of Yap, or anti-Yap antibodies and antigen-binding portions thereof, are administered to a subject who had a surgery to remove heterotopic ossification tissues.

The methods of the present invention further comprising selecting a subject. In some embodiments, the subject suffers from or is at risk of developing heterotopic ossification. In some embodiments, the subject suffers from or is at risk of developing a genetic heterotopic ossification disorder. In some embodiments, the subject suffers from or is at risk of developing a trauma-induced heterotopic ossification disorder.

To practice the method disclosed herein, an effective amount of the pharmaceutical composition described above can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the agents that inhibit the expression and/or activity of Yap can be aerosolized using a fluorocarbon formulation and a metered dose inhaler, or inhaled as a lyophilized and milled powder.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an agent that inhibits the expression and/or activity of Yap is administered via site-specific or targeted local delivery techniques. Examples of site-specific or targeted local delivery techniques include topical administration, e.g., directly to the site of injure.

The particular dosage regimen, e.g., dose, timing and repetition, used in the method described herein will depend on the particular subject and that subject's medical history.

“An effective amount” as used herein refers to the amount of each active agent required to confer a therapeutic effect on the subject, either alone or in combination with one or more other active agents. For example, an effective amount refers to the amount of an agent inhibiting the expression and/or activity of Yap of the present disclosure which is sufficient to achieve a biological effect, e.g., a decrease in the expression and/or activity of Yap, a decrease in the expression and/or activity of Shh, or a reduction of heterotopic ossification in the cell.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

In some embodiments, in the context of a decrease in the level of Yap in a cell, the decrease is at least 1-fold, 1.2-fold, 1.5-fold, 2-fold, 2.5-fold, 3-fold, 4-fold, 5-fold, 6-fold, 7-fold, 8-fold, 9-fold, or 10-fold or more (or any range bracketed by any of the values), compared to a control level of Yap. In one embodiment, the decrease in the level of Yap in the cell is a decrease in a range of 1-fold to 3-fold, 1.2-fold to 10-fold, 2-fold to 9-fold, 3-fold to 8-fold, 4-fold to 7-fold, 2-fold to 7-fold, etc. compared to the control level of Yap.

In some embodiments, in the context of a decrease in the expression and/or activity level of Yap in the cell after the administering step, the decrease is detectable within 4 hours, 24 hours, 48 hours, 7 days, 14 days, 21 days, 28 days or 30 days (or any time range bracketed by any of the listed duration of times) after the administering step. In one embodiment, a decrease in the expression and/or activity level of Yap in the cell after the administering step is detectable for at least 5 days, 7 days, 14 days, 21 days, 28 days, or 30 days (or any time range bracketed by any of the listed duration of times) after the administering step. In one embodiment, a decrease in the expression and/or activity level of Yap in the cell after the administering step is at least 1-fold, 1.2-fold, 1.5-fold, 2-fold, 2.5-fold, 3-fold, 4-fold, 5-fold, 6-fold, 7-fold, 8-fold, 9-fold, or 10-fold or more (or any range bracketed by any of the values), compared to the level of Yap in the cell before the administering step. In one embodiment, a decrease in the expression and/or activity level of Yap in the cell after the administering step is a decrease in a range of 1-fold to 3-fold, 1.2-fold to 10-fold, 2-fold to 9-fold, 3-fold to 8-fold, 4-fold to 7-fold, 2-fold to 7-fold, etc., compared to the level of Yap in the cell before the administering step.

In some embodiment, in the context of a decrease in the level of Yap in the circulation after the administering step, a decrease is detectable within 4 hours, 24 hours, 48 hours, 7 days, 14 days, 21 days, 28 days, or 30 days (or any time range bracketed by any of the listed duration of times) after the administering step. In one embodiment, a decrease in the level of Yap in the circulation after the administering step is detectable for at least 5 days, 7 days, 14 days, 21 days, 28 days, or 30 days (or any time range bracketed by any of the listed duration of times) after the administering step. In one embodiment, a decrease in the level of Yap in the circulation after the administering step is at least 1-fold, 2-fold, 3-fold, 5-fold, 10-fold, 15-fold, 20-fold, 25-fold, 30-fold, 35-fold, 40-fold, 45-fold, or 50-fold or more (or any range bracketed by any of the values), compared to the level of Yap in the circulation before the administering step. In one embodiment, a decrease in the level of Yap in the circulation after the administering step is a decrease in a range of 1-fold to 3-fold, 1.2-fold to 10-fold, 2-fold to 9-fold, 3-fold to 8-fold, 4 fold to 7-fold, 2-fold to 7-fold, etc., compared to the level of Yap in the circulation before the administering step.

As discussed above, in some embodiments, in the context of administration of an agent that inhibits expression and/or activity of Yap to a subject, an effective amount is an amount effective to reduce heterotopic ossification in the subject compared with a control subject. In some embodiments, heterotopic ossification in the subject treated with an effective amount of the agent is decreased by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, etc., as compared with a control subject that is not treated with an effective amount of the agent.

In some embodiments, in the context of administration of an agent that inhibits expression and/or activity of Yap to a subject an effective amount is an amount effective to reduce heterotopic ossification in the subject.

Empirical considerations, such as the half-life, generally will contribute to the determination of the dosage. For example, antibodies and antigen-binding portions thereof that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a disease/disorder associated with heterotopic ossification. Alternatively, sustained continuous release formulations of a Yap inhibitor, e.g., small molecule inhibitors of Yap, or an anti-Yap antibody, or antigen-binding portion thereof, may be appropriate. Various formulations and devices for achieving sustained release would be apparent to the skilled artisan and are within the scope of this disclosure.

In one example, dosages for an agent that inhibits expression and/or activity of Yap, as described herein may be determined empirically in individuals who have been given one or more administration(s) of the Yap inhibitor. Individuals are given incremental dosages of the antagonist. To assess efficacy of the antagonist, an indicator of the disease/disorder can be followed.

Generally, for administration of any of the inhibitors described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a disease or disorder associated with heterotopic ossification, or a symptom thereof. An exemplary dosing regimen comprises administering an initial dose of about 2 mg/kg, followed by a weekly maintenance dose of about 1 mg/kg of the antibody, or antigen binding fragment thereof, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, every 4 months, every 5 months, every 6 months, every 8 months, every 10 months, every year, or longer. The progress of this therapy is easily monitored by conventional techniques and assays. The dosing regimen (including the antibody used) can vary over time.

In some embodiments, the administration of any of the agents that inhibit the expression and/or activity of Yap, described herein, comprises a single dose. In some embodiments, the administration of any of the Yap inhibitors, described herein comprises multiple doses (e.g., at least 2, 3, 4, 5, 6, 7, 8, 9, or 10 doses). Administering may comprise more than two doses. In some embodiments, the administration comprises at least a first dose and a second dose of a therapeutically effective amount of the Yap inhibitor. In one embodiment, the first dose and the second dose are administered to the subject at least about 4 weeks apart, 6 weeks apart, 8 weeks apart, or 12 weeks apart.

In some embodiments, for an adult patient of normal weight, doses ranging from about 0.3 to 5.00 mg/kg may be administered. The particular dosage regimen, e.g., dose, timing and repetition, will depend on the particular individual and that individual's age, sex and medical history, as well as the properties of the individual agents (such as the half-life of the agent, and other relevant considerations).

For the purpose of the present disclosure, the appropriate dosage of an agent that inhibits the expression and/or activity of Yap, will depend on the specific inhibitors (or compositions thereof) employed, the type and severity of the disease/disorder, whether the inhibitor is administered for preventive or therapeutic purposes, previous therapy, the patient's clinical history and response to the antagonist, and the discretion of the attending physician. In some embodiments, a clinician will administer a Yap inhibitor, until a dosage is reached that achieves the desired result. Administration of a Yap inhibitor can be continuous or intermittent, depending, for example, upon the recipient's physiological condition, whether the purpose of the administration is therapeutic or prophylactic, and other factors known to skilled practitioners. The administration of a Yap inhibitor may be essentially continuous over a preselected period of time or may be in a series of spaced dose, e.g., either before, during, or after developing a disease or disorder associated with heterotopic ossification.

As used herein, the term “treating” refers to the application or administration of a composition including one or more active agents to a subject, who has a disease/disorder associated with heterotopic ossification, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease/disorder.

The invention encompasses pharmaceutical compositions and related methods used as combination therapies for treating subjects who may benefit from Yap inhibition in vivo. In any of these embodiments, such subjects may receive combination therapies that include a first composition comprising at least one agent that inhibits the expression and/or activity of Yap, e.g., small molecule inhibitors of Yap, described herein, in conjunction with a second composition comprising at least one additional therapeutic intended to treat the same or overlapping disease or clinical condition. The first and second compositions may both act on the same cellular target, or discrete cellular targets. In some embodiments, the first and second compositions may treat or alleviate the same or overlapping set of symptoms or aspects of a disease or clinical condition. In some embodiments, the first and second compositions may treat or alleviate a separate set of symptoms or aspects of a disease or clinical condition. Such combination therapies may be administered in conjunction with each other. The phrase “in conjunction with,” in the context of combination therapies, means that therapeutic effects of a first therapy overlaps temporarily and/or spatially with therapeutic effects of a second therapy in the subject receiving the combination therapy. Thus, the combination therapies may be formulated as a single formulation for concurrent administration, or as separate formulations, for sequential administration of the therapies.

In preferred embodiments, combination therapies produce synergistic effects in the treatment of a disease. The term “synergistic” refers to effects that are greater than additive effects (e.g., greater efficacy) of each monotherapy in aggregate.

In some embodiments, combination therapies comprising a pharmaceutical composition described herein produce efficacy that is overall equivalent to that produced by another therapy (such as monotherapy of a second agent) but are associated with fewer unwanted adverse effect or less severe toxicity associated with the second agent, as compared to the monotherapy of the second agent. In some embodiments, such combination therapies allow lower dosage of the second agent but maintain overall efficacy. Such combination therapies may be particularly suitable for patient populations where a long-term treatment is warranted and/or involving pediatric patients.

Accordingly, the invention provides pharmaceutical compositions and methods for use in combination therapies for the treatment of heterotopic ossification diseases or disorders. In some embodiments, the methods or the pharmaceutical compositions further comprise a second therapy. The second therapy may diminish or treat at least one symptom(s) associated with the targeted disease. The first and second therapies may exert their biological effects by similar or unrelated mechanisms of action; or either one or both of the first and second therapies may exert their biological effects by a multiplicity of mechanisms of action.

It should be understood that the pharmaceutical compositions described herein may have the first and second therapies in the same pharmaceutically acceptable carrier or in a different pharmaceutically acceptable carrier for each described embodiment. It further should be understood that the first and second therapies may be administered simultaneously or sequentially within described embodiments.

The one or more agents that inhibit the expression and/or activity of Yap of the invention may be used in combination with one or more of additional therapeutic agents. Examples of the additional therapeutic agents which can be used with an agent of the invention include, but are not limited to, nonsteroidal anti-inflammatory drugs (NSAIDS), chemotherapeutic agents, immunotherapeutic agents, immunosuppressive agents, and the like. Other agents useful in the combination therapies of the invention include activators of Yap phosphorylation and inhibitors of Taz, which plays redundant roles with Yap. Such combination therapies may advantageously utilize lower dosages of the administered therapeutic agents, thus avoiding possible toxicities or complications associated with the various monotherapies.

Exemplary inhibitors of Taz include, but are not limited to, verteporfin (VP), (R)-PFI 2 hydrochloride, CA3(CIL56), XMU MP 1 and YAP/TAZ inhibitor-1 as described in WO2017058716A1, the entire content of which is incorporated herein by reference.

Exemplary activators of Yap phosphorylation include, but are not limited to, TAO Kinase (Thousand And One Amino Acid Protein Kinase), MST1/2 (mammalian Ste20-like kinases 1/2), and LATS1/2 (large tumor suppressor kinases 1/2), all are responsible for phosphorylation of YAP, leading to degradation of YAP and/or cytoplasmic retention of YAP.

Exemplary nonsteroidal anti-inflammatory agents (NSAIDS) include, but are not limited to, aspirin, celecoxib, diclofenac, ibuprofen, indomethacin, ketoprofen, naproxen, oxaprozin, piroxicam.

Chemotherapeutic agents include, for example, alkylating agents (e.g., cyclophosphamide, iphosphamide and the like), metabolism antagonists (e.g., methotrexate, 5-fluorouracil and the like), anticancer antibiotics (e.g., mitomycin, adriamycin and the like), vegetable-derived anticancer agents (e.g., vincristine, vindesine, taxol and the like), cisplatin, carboplatin, etoposide and the like. Among these substances, 5-fluorouracil derivatives such as furtulon and neofurtulon are preferred.

Immunotherapeutic agents include, for example, microorganisms or bacterial components (e.g., muramyl dipeptide derivative, picibanil and the like), polysaccharides having immune potentiating activity (e.g., lentinan, sizofilan, krestin and the like), cytokines obtained by a gene engineering technology (e.g., interferon, interleukin (IL) and the like), colony stimulating factors (e.g., granulocyte colony stimulating factor, erythropoetin and the like) and the like, among these substances, those preferred are IL-1, IL-2, IL-12 and the like.

Immunosuppressive agents include, for example, calcineurin inhibitor/immunophilin modulators such as cyclosporine (Sandimmune, Gengraf, Neoral), tacrolimus (Prograf, FK506), ASM 981, sirolimus (RAPA, rapamycin, Rapamune), or its derivative SDZ-RAD, glucocorticoids (prednisone, prednisolone, methylprednisolone, dexamethasone and the like), purine synthesis inhibitors (mycophenolate mofetil, MMF, CellCept®, azathioprine, cyclophosphamide), interleukin antagonists (basiliximab, daclizumab, deoxyspergualin), lymphocyte-depleting agents such as antithymocyte globulin (Thymoglobulin, Lymphoglobuline), anti-CD3 antibody (OKT3), and the like.

In some embodiments, second agents suitable for administration as a combination therapy in conjunction with the agents that inhibit the expression and/or activity of Yap are inhibitors of of an protein that interacts with Yap, e.g., Shh, or CDK7, as described herein.

In some embodiments, second agents suitable for administration as a combination therapy in conjunction with the agents described herein are modulators (e.g., agonists and antagonists) of certain members of the Hedgehog pathway.

Any of the above-mentioned agents can be administered in combination with the agent that inhibits the expression and/or activity of Yap to treat a heterotopic ossification disease or disorder.

### Screening Methods

In certain aspect, the presentation provides a method for identifying a compound that inhibits heterotopic ossification (HO) of a cell. The method comprises providing a cellular indicator composition, contacting the indicator composition with a test compound, determining the effect of the compound on the expression and or activity of YAP in the indicator composition, wherein a decrease in the expression and/or activity of Yap indicates that the test compound inhibits HO, thereby identifying a compound that inhibits HO of the cell.

In some embodiments, the indicator composition comprises a mesenchymal progenitor cell, a mesenchymal stem cell, a mesenchyme derived differentiated cell or a heterotopic ossified cell. The cells suitable for the methods of the invention may comprise an inactive Gnas gene, or a constitutively active caALK2 gene. Alternatively, the cell may comprise an ACVR^{R206H} gene.

In addition to have an effect on the expression and/or activity of Yap in the indicator composition, the test compound may also have an effect on the expression and/or activity of several other markers, e.g., Osx, Col1a1, Opn, Runx2, Shh, Ihh, Ctgf, Cyr61, Ankdr1, Ptch1, Gli1, Hip, Taz, Tgfβ1, Tgfβ2 and Tgfβ3. Accordingly, the methods of the present invention further comprise determining the expression and/or activity of these markers in order to identify a compound that inhibits HO of a cell.

Other well known methods that may be used to identify small molecules from libraries which bind Yap, or any other protein that interacts with Yap, for example, Shh, CDK7, or any other components of the Hedgehog pathway, e.g., Dhh, Ihh, Patched (Ptch1, Ptch2), Gli1, Gli2, Gli3, and Smoothened (Smo), include methods that utilize libraries in which the library members are tagged with an identifying label, that is, each label present in the library is associated with a discreet compound structure present in the library, such that identification of the label tells the structure of the tagged molecule. One approach to tagged libraries utilizes oligonucleotide tags, as described, for example, in PCT Publication No. WO 2005/058479 A2 (the Direct Select technology) and in U.S. Pat. Nos. 5,573,905; 5,708,153; 5,723,598, 6,060,596 published PCT applications WO 93/06121; WO 93/20242; WO 94/13623; WO 00/23458; WO 02/074929 and WO 02/103008, and by Brenner and Lerner (1992) Proc. Natl. Acad. Sci. USA 89: 5381-5383; Nielsen and Janda (1994) Methods Enzymol. 6: 361-371; and Nielsen et al. (1993) J. Am. Chem. Soc. 115: 9812-9813, the entire contents of each of which are incorporated herein by reference in their entirety. Such tags can be amplified, using for example, polymerase chain reaction, to produce many copies of the tag and identify the tag by sequencing. The sequence of the tag then identifies the structure of the binding molecule, which can be synthesized in pure form and tested for activity.

Preparation and screening of combinatorial chemical libraries is well known to those skilled in the art. Such combinatorial chemical libraries, which may be used to identify moieties of the invention, include, but are not limited to, peptide libraries (see, e.g., U.S. Pat. No. 5,010,175, Furka (1991) Int. J Pept. Prot. Res. 37: 487 493 and Houghton et al. (1991) Nature 354: 84 88). Other chemistries for generating chemical diversity libraries are well known in the art and can be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al. (1993) Proc. Nat. Acad. Sci. USA 90: 6909-6913), vinylogous polypeptides (Hagihara et al. (1992) J. Am. Chem. Soc. 114: 6568), nonpeptidal peptidomimetics with glucose scaffolding (Hirschmann et al. (1992) J. Am. Chem. Soc. 114: 9217 9218), analogous organic syntheses of small compound libraries (Chen et al. (1994) J. Am. Chem. Soc. 116: 2661), oligocarbamates (Cho et al. (1993) Science 261: 1303), and/or peptidyl phosphonates (Campbell et al. (1994) J. Org. Chem. 59: 658), nucleic acid libraries (see Ausubel, Berger and Russell & Sambrook, all supra), peptide nucleic acid libraries (see, e.g., U.S. Pat. No. 5,539,083), carbohydrate libraries (see, e.g., Liang et al. (1996) Science 274: 1520 1522 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like). Each of the foregoing publications is incorporated herein by reference. Public databases are also available and are commonly used for small molecule screening, e.g., PubChem (pubchem.ncbi.nlm.nih.gov), Zinc (Irwin and Shoichet (2005) J. Chem. Inf. Model. 45: 177-182), and ChemBank (Seiler et al. (2008) Nucleic Acids Res. 36: D351-D359).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.). Moreover, since screening methodologies are so well defined, it is common to contract specialist firms to identify particular compounds for a target of interest (e.g., BioFocus DPI (biofocus.com), and Quantum Lead (q-lead.com)).

Other methods of selecting small molecules which are well known in the art, and may be applied to the methods of the present invention are Huang and Schreiber (1997) Proc. Natl. Acad. Sci. USA 94: 13396-13401; Hung et al. (2005) Science 310: 670-674; Zhang et al. (2007) Proc. Natl. Acad. Sci. USA 104: 4606-4611; or any of the methods reviewed in Gordon (2007) ACS Chem. Biol. 2: 9-16, all of which are incorporated herein by reference in their entirety.

In addition to experimental screening methods, small molecules of the invention may be selected using virtual screening methods. Virtual screening technologies predict which small molecules from a library will bind to a protein, or a specific epitope therein, using statistical analysis and protein docking simulations. Most commonly, virtual screening methods compare the three-dimensional structure of a protein to those of small molecules in a library. Different strategies for modeling protein-molecule interactions are used, although it is common to employ algorithms that simulate binding energies between atoms, including hydrogen bonds, electrostatic forces, and van der Waals interactions. Typically, virtual screening methods can scan libraries of more than a million compounds and return a short list of small molecules that are likely to be strong binders. Several reviews of virtual screening methods are available, detailing the techniques that may be used to identify small molecules of the present invention (Engel et al. (2008) J. Am. Chem. Soc. 130, 5115-5123; McInnes (2007) Curr. Opin. Chem. Biol. 11: 494-502; Reddy et al. (2007) Curr. Protein Pept. Sci. 8: 329-351; Muegge and Oloff (2006) Drug Discov. Today 3: 405-411; Kitchen et al. (2004) Nat. Rev. Drug Discov. 3, 935-949). Further examples of small molecule screening can be found in U.S. 2005/0124678, which is incorporated herein by reference.

In some embodiments, small molecules identified as “hits” (e.g., small molecules that demonstrate activity in a method described herein) in a first screen are selected and optimized by being systematically altered, e.g., using rational design, to optimize binding affinity, avidity, specificity, or other parameter. Such potentially optimized structures can also be screened using the methods described herein. Thus, in one embodiment, the invention includes screening a first library of small molecules using a method described herein, identifying one or more hits in that library, subjecting those hits to systematic structural alteration to create one or more second generation compounds structurally related to the hit, and screening the second generation compound. Additional rounds of optimization can be used to identify a small molecule with a desirable therapeutic profile.

Small molecules identified as hits can be considered candidate therapeutic compounds, useful in treating heterotopic ossification disorders described herein. Thus, the invention also includes compounds identified as “hits” by a method described herein, and methods for their administration and use in the treatment, prevention, or delay of development or progression of a disease described herein.

In one embodiment, the small molecule inhibitor of Yap acts directly on Yap and inhibits its function. In another embodiment, the inhibitor of Yap acts indirectly, e.g., through another molecule which interacts with Yap, e.g., Shh or CDK7, thereby decreasing Yap expression and/or activity.

### III. Agents that Inhibit the Expression and/or Activity of Yap

As described above, the invention includes, in some embodiments, agents that inhibit the expression and/or activity of Yap in order to inhibit heterotopic ossification (HO) in a cell. Examples of such agents that may be used in the methods and compositions described herein are provided below, and include, but are not limited to, inhibitory nucleic acids, RNA-guided nucleases, small molecule inhibitors, peptic molecules, inhibitory fusion proteins and antagonist antibodies, or antigen-binding fragment therefore. An inhibitory agent (i.e., inhibitor) can be a nucleic acid, a polypeptide, an antibody, or a small molecule compound. In some embodiments, the inhibitor functions at a level of transcription and mRNA stability. In other embodiments, the inhibitor acts at a level of translation, protein stability/degradation, protein modification, and protein binding.

### Inhibitory Nucleic Acids

In one embodiment, the methods described herein include targeting Yap using inhibitory nucleic acids. A nucleic acid inhibitor can encode a small interference RNA (e.g., an RNAi agent) that targets the Yap gene, or a gene encoding for another protein that interacts with Yap, e.g., Shh or CDK7, and inhibits the expression or activity. The term “RNAi agent” refers to an RNA, or analog thereof, having sufficient sequence complementarity to a target RNA to direct RNA interference. Examples also include a DNA that can be used to make the RNA.

RNA Interference: RNA interference (RNAi) refers to a sequence-specific or selective process by which a target molecule (e.g., a target gene, protein or RNA) is down-regulated. Generally, an interfering RNA (“RNAi”) is a double stranded short-interfering RNA (siRNA), short hairpin RNA (shRNA), or single-stranded micro-RNA (miRNA) that results in catalytic degradation of specific mRNAs, and also can be used to lower or inhibit gene expression. RNA interference (RNAi) is a process whereby double-stranded RNA (dsRNA) induces the sequence-specific regulation of gene expression in animal and plant cells and in bacteria (Aravin and Tuschl, FEBS Lett. 26:5830-5840 (2005); Herbert et al., Curr. Opin. Biotech. 19:500-505 (2008); Hutvagner and Zamore, Curr. Opin. Genet. Dev., 12: 225-232 (2002); Sharp, Genes Dev., 15:485-490 (2001); Valencia-Sanchez et al. Genes Dev. 20:515-524 (2006)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), by microRNA (miRNA), functional small-hairpin RNA (shRNA), or other dsRNAs which are expressed in vivo using DNA templates with RNA polymerase II or III promoters (Zeng et al., Mol. Cell 9:1327-1333 (2002); Paddison et al., Genes Dev. 16:948-958 (2002); Denti, et al., Mol. Ther. 10:191-199 (2004); Lee et al., Nature Biotechnol. 20:500-505 (2002); Paul et al., Nature Biotechnol. 20:505-508 (2002); Rossi, Human Gene Ther. 19:313-317 (2008); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Scherer et al., Nucleic Acids Res. 35:2620-2628 (2007); Sui et al., Proc. Natl. Acad. Sci. USA 99(6):5515-5520 (2002).)

siRNA Molecules: The term “short interfering RNA” or “siRNA” (also known as “small interfering RNAs”) refers to an RNA agent, preferably a double-stranded agent, of about 10-50 nucleotides in length, preferably between about 15-25 nucleotides in length, more preferably about 17, 18, 19, 20, 21, 22, 23, 24, or 25 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery.

In general, the methods described herein can use dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, or can be transcribed in vitro or in vivo, e.g., shRNA, from a DNA template. The dsRNA molecules can be designed using any method known in the art. Negative control siRNAs should not have significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The methods described herein can use both siRNA and modified siRNA derivatives, e.g., siRNAs modified to alter a property such as the specificity and/or pharmacokinetics of the composition, for example, to increase half-life in the body, e.g., crosslinked siRNAs. Thus, the invention includes methods of administering siRNA derivatives that include siRNA having two complementary strands of nucleic acid, such that the two strands are crosslinked. The oligonucleotide modifications include, but are not limited to, 2′-O-methyl, 2′-fluoro, 2′-O-methyoxyethyl and phosphorothioate, boranophosphate, 4′-thioribose. (Wilson and Keefe, Curr. Opin. Chem. Biol. 10:607-614 (2006); Prakash et al., J. Med. Chem. 48:4247-4253 (2005); Soutschek et al., Nature 432:173-178 (2004)).

In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The inhibitory nucleic acid compositions can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.:47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles). The inhibitory nucleic acid molecules can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P, or other appropriate isotope.

siRNA Delivery: Direct delivery of siRNA in saline or other excipients can silence target genes in tissues, such as the eye, lung, and central nervous system (Bitko et al., Nat. Med. 11:50-55 (2005); Shen et al., Gene Ther. 13:225-234 (2006); Thakker et al., Proc. Natl. Acad. Sci. U.S.A. (2004)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)).

Liposomes and nanoparticles can also be used to deliver siRNA into animals. Delivery methods using liposomes, e.g. stable nucleic acid-lipid particles (SNALPs), dioleoyl phosphatidylcholine (DOPC)-based delivery system, as well as lipoplexes, e.g. Lipofectamine 2000, TransIT-TKO, have been shown to effectively repress target mRNA (de Fougerolles, Human Gene Ther. 19:125-132 (2008); Landen et al., Cancer Res. 65:6910-6918 (2005); Luo et al., Mol. Pain 1:29 (2005); Zimmermann et al., Nature 441:111-114 (2006)). Conjugating siRNA to peptides, RNA aptamers, antibodies, or polymers, e.g. dynamic polyconjugates, cyclodextrin-based nanoparticles, atelocollagen, and chitosan, can improve siRNA stability and/or uptake. (Howard et al., Mol. Ther. 14:476-484 (2006); Hu-Lieskovan et al., Cancer Res. 65:8984-8992 (2005); Kumar, et al., Nature 448:39-43; McNamara et al., Nat. Biotechnol. 24:1005-1015 (2007); Rozema et al., Proc. Natl. Acad. Sci. U.S.A. 104:12982-12987 (2007); Song et al., Nat. Biotechnol. 23:709-717 (2005); Soutschek (2004), supra; Wolfrum et al., Nat. Biotechnol. 25:1149-1157 (2007)).

Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002), supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., Proc. Natl. Acad. Sci. USA 99(22):14236-40 (2002)).

Stable siRNA Expression: Synthetic siRNAs can be delivered into cells, e.g., by direct delivery, cationic liposome transfection, and electroporation. However, these exogenous siRNA typically only show short term persistence of the silencing effect (4-5 days). Several strategies for expressing siRNA duplexes within cells from recombinant DNA constructs allow longer-term target gene suppression in cells, including mammalian Pol II and III promoter systems (e.g., H1, U1, or U6/snRNA promoter systems (Denti et al. (2004), supra; Tuschl (2002), supra); capable of expressing functional double-stranded siRNAs (Bagella et al., J. Cell. Physiol. 177:206-213 (1998); Lee et al. (2002), supra; Miyagishi et al. (2002), supra; Paul et al. (2002), supra; Scherer et al. (2007), supra; Yu et al. (2002), supra; Sui et al. (2002), supra).

Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into the cells with a vector expression T7 RNA polymerase (Jacque (2002), supra).

In another embodiment, siRNAs can be expressed in a miRNA backbone which can be transcribed by either RNA Pol II or III. MicroRNAs are endogenous noncoding RNAs of approximately 22 nucleotides in animals and plants that can post-transcriptionally regulate gene expression (Bartel, Cell 116:281-297 (2004); Valencia-Sanchez et al., Genes & Dev. 20:515-524 (2006)). One common feature of miRNAs is that they are excised from an approximately 70 nucleotide precursor RNA stem loop by Dicer, an RNase III enzyme, or a homolog thereof By substituting the stem sequences of the miRNA precursor with the sequence complementary to the target mRNA, a vector construct can be designed to produce siRNAs to initiate RNAi against specific mRNA targets in mammalian cells. When expressed by DNA vectors containing polymerase II or III promoters, miRNA designed hairpins can silence gene expression (McManus (2002), supra; Zeng (2002), supra).

Antisense: An “antisense” nucleic acid can include a nucleotide sequence that is complementary to a “sense” nucleic acid encoding a protein, e.g., complementary to the coding strand of a double-stranded cDNA molecule or complementary to a target mRNA sequence. The antisense nucleic acid can be complementary to an entire coding strand of a target sequence, or to only a portion thereof (for example, the coding region of a target gene). In another embodiment, the antisense nucleic acid molecule is antisense to a “noncoding region” of the coding strand of a nucleotide sequence encoding the selected target gene (e.g., the 5′ and 3′ untranslated regions).

An antisense nucleic acid can be designed such that it is complementary to the entire coding region of a target mRNA but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Based upon the sequences disclosed herein, e.g., sequences relating to Yap or Shh, one of skill in the art can easily choose and synthesize any of a number of appropriate antisense molecules for use in accordance with the present invention. For example, a “gene walk” comprising a series of oligonucleotides of 15-30 nucleotides spanning the length of a target nucleic acid can be prepared, followed by testing for inhibition of target gene expression. Optionally, gaps of 5-10 nucleotides can be left between the oligonucleotides to reduce the number of oligonucleotides synthesized and tested.

The antisense nucleic acid molecules of the invention are typically administered to a subject (e.g., by direct injection at a tissue site), or generated in situ such that they hybridize with or bind to cellular mRNA and/or genomic DNA encoding a target protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In some embodiments, the antisense nucleic acid is a morpholino oligonucleotide (see, e.g., Heasman, Dev. Biol. 243:209-14 (2002); Iversen, Curr. Opin. Mol. Ther. 3:235-8 (2001); Summerton, Biochim. Biophys. Acta. 1489:141-58 (1999).

Target gene expression can be inhibited by targeting nucleotide sequences complementary to a regulatory region, e.g., promoters and/or enhancers) to form triple helical structures that prevent transcription of the target gene in target cells. See generally, Helene, C. Anticancer Drug Des. 6:569-84 (1991); Helene, C. Ann. N.Y. Acad. Sci. 660:27-36 (1992); and Maher, Bioassays. 14:807-15 (1992). The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

### Fusion Proteins and RNA-Guided Nucleases

In another aspect of the invention, the agent that inhibits the expression and/or activity of Yap is a fusion protein.

As used herein, a “chimeric protein” or “fusion protein” comprises all or part (preferably a biologically active part) of a first protein operably linked to a heterologous second polypeptide (i.e., a polypeptide other than the first protein). Within the fusion protein, the term “operably linked” is intended to indicate that the first protein or segment thereof and the heterologous polypeptide are fused in-frame to each other. The heterologous polypeptide can be fused to the amino-terminus or the carboxyl-terminus of the first protein or segment.

In some embodiments, the fusion proteins of the invention include Yap fused to an effector molecule. In some embodiments, the fusion proteins of the invention include a protein that interacts with Yap, e.g., Shh or CDK7, fused to an effector molecule. Exemplary effector molecules include are described below and in some embodiments include, for example, nucleases, physical blockers, epigenetic recruiters, e.g., a transcriptional repressor, and epigenetic CpG modifiers, e.g., a DNA methylase, a DNA demethylase, a histone modifying agent, or a histone deacetylase, and combinations of any of the foregoing.

In one embodiment, the agents used to inhibit Yap expression and/or activity are based on CRISPR technology and are RNA-guided nucleases targeting Yap, or any other protein that interacts with Yap, e.g., Shh or CDK7.

The clustered, regularly interspaced, short palindromic repeat (CRISPR) technology is included in the invention as an approach for generating RNA-guided nuclease with customizable specificities for targeted genome editing. Genome editing mediated by these nucleases has been used to rapidly, easily and efficiently modify endogenous genes in a wide variety of biomedically important cell types and in organisms that have traditionally been challenging to manipulate genetically.

In general, the term “CRISPR system” refers collectively to transcripts and other/elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In embodiments of the invention the terms guide sequence and guide RNA are used interchangeably. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides (e.g., DNA or RNA of YYap, Shh, or CDK7). In some embodiments, a target sequence is located in the nucleus or cytoplasm of a cell.

In preferred embodiments of the invention, the CRISPR/Cas system is a type II CRISPR system and the Cas enzyme is Cas9, which catalyzes DNA cleavage. Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence NGG following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defense in bacteria and archae, Mol. Cell 2010, January 15; 37(1): 7.

The type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. Several aspects of the CRISPR system can be further improved to increase the efficiency and versatility of CRISPR targeting. Optimal Cas9 activity may depend on the availability of free Mg2+ at levels higher than that present in the mammalian nucleus (see e.g. Jinek et al., 2012, Science, 337:816), and the preference for an NGG motif immediately downstream of the protospacer restricts the ability to target on average every 12-bp in the human genome.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

The expression of a target polynucleotide can be modified by allowing a CRISPR complex to bind to the polynucleotide, wherein the CRISPR complex comprises a CRISPR enzyme complexed with a guide sequence hybridized to a target sequence within said polynucleotide, e.g., an RNA-guided nuclease targeting Yap, Shh or CDK7, wherein said guide sequence is linked to a tracr mate sequence which in turn hybridizes to a tracr sequence. In some embodiment, binding of CRISPR complex to a target polynucleotide results in an increased expression of the target polynucleotide. In another embodiment, binding of CRISPR complex to a target polynucleotide results in a decreased expression of the target polynucleotide (e.g., DNA or RNA of Yap, Shh or CDK7).

In some embodiment, the fusion protein may comprise an effector, such as a nuclease, e.g., a Cas9, e.g., a wild type Cas9, a nickase Cas9 (e.g., Cas9 D10A), a dead Cas9 (dCas9), eSpCas9, Cpfl, C2C1, or C2C3, or a nucleic acid encoding such a nuclease. The choice of nuclease and gRNA(s) is determined by whether the targeted mutation is a deletion, substitution, or addition of nucleotides, e.g., a deletion, substitution, or addition of nucleotides to a targeted sequence. Fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be linked to the polypeptide to guide the composition to specific DNA sites by one or more RNA sequences (e.g., DNA recognition elements including, but not restricted to zinc finger arrays, sgRNA, TAL arrays, peptide nucleic acids described herein) to modulate activity and/or expression of one or more target nucleic acids sequences (e.g., to methylate or demethylate a DNA sequence).

In one embodiment, a fusion protein of the invention may comprise an effector molecule comprising, for example, a CRISPER associated protein (Cas) polypeptide, or fragment thereof, (e.g., a Cas9 polypeptide, or fragment thereof) and an epigenetic recruiter or an epigenetic CpG modifier.

In one embodiment, a suitable Cas polypeptide is an enzymatically inactive Cas polypeptide, e.g., a “dead Cas polypeptide” or “dCas” polypeptide Exemplary Cas polypeptides that are adaptable to the methods and compositions described herein are described below. Using methods known in the art, a Cas polypeptide can be fused to any of a variety of agents and/or molecules as described herein; such resulting fusion molecules can be useful in various disclosed methods.

In one aspect, the invention includes a composition comprising a protein comprising a domain, e.g., an effector, that acts on DNA (e.g., a nuclease domain, e.g., a Cas9 domain, e.g., a dCas9 domain; a DNA methyltransferase, a demethylase, a deaminase), in combination with at least one guide RNA (gRNA) or antisense DNA oligonucleotide that targets the protein to site-specific target sequence, wherein the composition is effective to alter, in a human cell, the expression of a target gene. In some embodiments, the enzyme domain is a Cas9 or a dCas9. In some embodiments, the protein comprises two enzyme domains, e.g., a dCas9 and a methylase or demethylase domain.

In one aspect, the invention includes a composition comprising a protein comprising a domain, e.g., an effector, that comprises a transcriptional control element (e.g., a nuclease domain, e.g., a Cas9 domain, e.g., a dCas9 domain; a transcriptional enhancer; a transcriptional repressor), in combination with at least one guide RNA (gRNA) or antisense DNA oligonucleotide that targets the protein to a site-specific target sequence, wherein the composition is effective to alter, in a human cell, the expression of a target gene. In some embodiments, the enzyme domain is a Cas9 or a dCas9. In some embodiments, the protein comprises two enzyme domains, e.g., a dCas9 and a transcriptional enhancer or transcriptional repressor domain.

As used herein, a “biologically active portion of an effector domain” is a portion that maintains the function (e.g. completely, partially, minimally) of an effector domain (e.g., a “minimal” or “core” domain).

The chimeric proteins described herein may also comprise a linker, e.g., an amino acid linker. In some aspects, a linker comprises 2 or more amino acids, e.g., one or more GS sequences. In some aspects, fusion of Cas9 (e.g., dCas9) with two or more effector domains (e.g., of a DNA methylase or enzyme with a role in DNA demethylation or protein acetyl transferase or deacetylase) comprises one or more interspersed linkers (e.g., GS linkers) between the domains. In some aspects, dCas9 is fused with 2-5 effector domains with interspersed linkers.

A variety of CRISPR associated (Cas) genes or proteins can be used in the present invention and the choice of Cas protein will depend upon the particular conditions of the method.

Specific examples of Cas proteins include class II systems including Cas1, Cas2, Cas3, Cas4, Cas5, Cash, Cas7, Cas8, Cas9, Cas10, Cpfl, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, the site-specific targeting moiety includes a sequence targeting polypeptide, such as an enzyme, e.g., Cas9. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Cryptococcus, a Corynebacterium, a Haemophilus, a Eubacterium, a Pasteurella, a Prevotella, a Veillonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the Cas protein is modified to deactivate the nuclease, e.g., nuclease-deficient Cas9, and to recruit transcription activators or repressors, e.g., the co-subunit of the E. coli Pol, VP64, the activation domain of p65, KRAB, or SID4X, to induce epigenetic modifications, e.g., histone acetyltransferase, histone methyltransferase and demethylase, DNA methyltransferase and enzyme with a role in DNA demethylation (e.g., the TET family enzymes catalyze oxidation of 5-methylcytosine to 5-hydroxymethylcytosine and higher oxidative derivatives).

For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence are required by Cas9 for DNA cleavage to occur; for Cpfl at least about 16 nucleotides of gRNA sequence is needed to achieve detectable DNA cleavage.

Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut the target DNA but interferes with transcription by steric hindrance. dCas9 can further be fused with a heterologous effector to repress (CRISPRi) or activate (CRISPRa) expression of a target gene. For example, Cas9 can be fused to a transcriptional silencer (e.g., a KRAB domain) or a transcriptional activator (e.g., a dCas9-VP64 fusion). A catalytically inactive Cas9 (dCas9) fused to Fokl nuclease (“dCas9-FokI”) can be used to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, Mass. 02139; addgene.org/crispr). A “double nickase” Cas9 that introduces two separate double-strand breaks, each directed by a separate guide RNA, is described as achieving more accurate genome editing by Ran et al.

(2013) Cell, 154: 1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpfl endonuclease and corresponding guide RNAs and PAM sites are disclosed in US Patent Application Publication 2016/0208243 A1.

In some embodiments, an effector comprises one or more components of a CRISPR system described hereinabove.

In some embodiments, suitable effectors for use in the agents, compositions, and methods of the invention include, for example, nucleases, physical blockers, epigenetic recruiters, e.g., a transcriptional enhancer or a transcriptional repressor, and epigenetic CpG modifiers, e.g., a DNA methylase, a DNA demethylase, a histone modifying agent, or a histone deacetylase, and combinations of any of the foregoing.

Exemplary effectors include ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3a, DNMT3b, DNMTL), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, hi stone-lysine-N-methyltransferase (Setdbl), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDMIA and lysine-specific histone demethylase 1 (LSD1), helicases such as DHX9, acetyltransferases, deacetylases (e.g., sirtuin 1, 2, 3, 4, 5, 6, or 7), kinases, phosphatases, DNA-intercalating agents such as ethidium bromide, sybr green, and proflavine, efflux pump inhibitors such as peptidomimetics like phenylalanine arginyl-naphthylamide or quinoline derivatives, nuclear receptor activators and inhibitors, proteasome inhibitors, competitive inhibitors for enzymes such as those involved in lysosomal storage diseases, zinc finger proteins, TALENs, specific domains from proteins, such as a KRAB domain, a VP64 domain, a p300 domain (e.g., p300 core domain), an MeCP2 domain, an MQ1 domain, a DNMT3a-3L domain a TET1 domain, and a TET2 domain, protein synthesis inhibitors, nucleases (e.g., Cpfl, Cas9, zinc finger nuclease), fusions of one or more thereof (e.g., dCas9-DNMT, dCas9-APOBEC, dCas9-UG1, dCas9-VP64, dCas9-p300 core, dCas9-KRAB, dCas9-KRAB-MeCP2, dCas9-MQ1, dCas9-DNMT3a-3L, dCAS9-TET1, dCAS9-TET2, and dCas9-MC/MN).

In some embodiments, a suitable nuclease for use in the agent, compositions, and methods of the invention comprises a transcription activator like effector nucleases (TALEN). In yet other embodiments, a suitable nuclease comprises a zinc finger protein.

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety.

TAL effectors are proteins secreted by Xanthomonas bacteria. The DNA binding domain contains a highly conserved 33-34 amino acid sequence with the exception of the 12th and 13th amino acids. These two locations are highly variable (Repeat Variable Diresidue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs.

The non-specific DNA cleavage domain from the end of the FokI endonuclease can be used to construct hybrid nucleases that are active in a yeast assay. These reagents are also active in plant cells and in animal cells. Initial TALEN studies used the wild-type FokI cleavage domain, but some subsequent TALEN studies also used FokI cleavage domain variants with mutations designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. The number of amino acid residues between the TALEN DNA binding domain and the FokI cleavage domain may be modified by introduction of a spacer (distinct from the spacer sequence) between the plurality of TAL effector repeat sequences and the FokI endonuclease domain. The spacer sequence may be 12 to 30 nucleotides.

The relationship between amino acid sequence and DNA recognition of the TALEN binding domain allows for designable proteins. In this case artificial gene synthesis is problematic because of improper annealing of the repetitive sequence found in the TALE binding domain. One solution to this is to use a publicly available software program (DNAWorks) to calculate oligonucleotides suitable for assembly in a two step PCR;
oligonucleotide assembly followed by whole gene amplification. A number of modular assembly schemes for generating engineered TALE constructs have also been reported. Both methods offer a systematic approach to engineering DNA binding domains that is conceptually similar to the modular assembly method for generating zinc finger DNA recognition domains.

Once the TALEN genes have been assembled they are inserted into plasmids; the plasmids are then used to transfect the target cell where the gene products are expressed and enter the nucleus to access the genome. TALENs can be used to edit genomes by inducing double-strand breaks (DSB), which cells respond to with repair mechanisms. In this manner, they can be used to correct mutations in the genome which, for example, cause disease.

As used herein, a “zinc finger polypeptide” or “zinc finger protein” is a protein that binds to DNA, RNA and/or protein, in a sequence-specific manner, by virtue of a metal stabilized domain known as a zinc finger. Zinc finger proteins are nucleases having a DNA cleavage domain and a DNA binding zinc finger domain. Zinc finger polypeptides may be made by fusing the nonspecific DNA. cleavage domain of an endonuclease with site-specific DNA binding zinc finger domains. Such nucleases are powerful tools for gene editing and can be assembled to induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

Zinc finger nucleases are chimeric enzymes made by fusing the nonspecific DNA. cleavage domain of the endonuclease FokI with site-specific DNA binding zinc finger domains. Due to the flexible nature of zinc finger proteins (ZFPs), ZFNs can be assembled that induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

In some embodiments, a suitable physical blocker for use in the agent, compositions, and methods of the invention comprises a gRNA, antisense DNA, or triplex forming oligonucleotide (which may target an expression control unit) steric block a transcriptional control element or anchoring sequence. The gRNA recognizes specific DNA sequences and further includes sequences that interfere with, e.g., a conjunction nucleating molecule sequence to act as a steric blocker. In some embodiments, the gRNA is combined with one or more peptides, e.g., S-adenosyl methionine (SAM), that acts as a steric presence. In other embodiments, a physical blocker comprises an enzymatically inactive Cas9 polypeptide, or fragment thereof (e.g., dCas9).

In one embodiment, an epigenetic recruiter activates or enhances transcription of a target gene, e.g., a gene that inhibits the expression and/or activity of Yap. In some embodiments, a suitable epigenetic recruiter for use in the agent, compositions, and methods of the invention comprises a VP64 domain or a p300 core domain.

In one embodiment, an epigenetic recruiter silences or represses transcription of a target gene, e.g., a gene that activates the expression and/or activity of Yap. In some embodiments, a suitable epigenetic recruiter for use in the agent, compositions, and methods of the invention comprises a KRAB domain, or an MeCP2 domain.

As used herein, “VP64” is a transcriptional activator composed of four tandem copies of VP16 (Herpes Simplex Viral Protein 16, amino acids 437-447*: DALDDFDLDML) connected with glycine-serine (GS) linkers. In one embodiment, the VP64 further comprises the transcription factors p65 and Rta at the C terminus.

As used herein, “p300 core domain” refers to the catalytic core of the human acetyltransferase p300.

As used herein, “KRAB” refers to a Kruppel associated box (KRAB) transcriptional repression domain present in human zinc finger protein-based transcription factors (KRAB zinc finger proteins).

As used herein, MeCp2” refers to methyl CpG binding protein 2 which represses transcription, e.g., by binding to a promoter comprising methylated DNA.

In one embodiment, an epigenetic CpG modifier methylates DNA and inactivates or represses transcription. In some embodiments, a suitable epigenetic CpG modifier for use in the agent, compositions, and methods of the invention comprises a MQ1 domain or a DNMT3a-3L domain.

In one embodiment, an epigenetic CpG modifier demethylates DNA and activates or stimulates transcription. In some embodiments, a suitable epigenetic recruiter for use in the agent, compositions, and methods of the invention comprises a TET1 or TET2 domain.

As used herein “TET1” refers to “ten-eleven translocation methylcytosine dioxygenase 1,” a member of the TET family of enzymes, encoded by the TET1 gene. TET1 is a dioxygenase that catalyzes the conversion of the modified DNA base 5-methylcytosine (5-mC) to 5 hydroxymethylcytosine (5-hmC) by oxidation of 5-mC in an iron and alpha-ketoglutarate dependent manner, the initial step of active DNA demethylation in mammals. Methylation at the C5 position of cytosine bases is an epigenetic modification of the mammalian genome which plays an important role in transcriptional regulation. In addition to its role in DNA demethylation, plays a more general role in chromatin regulation. Preferentially binds to CpG-rich sequences at promoters of both transcriptionally active and Polycomb-repressed genes. Involved in the recruitment of the O-GlcNAc transferase OGT to CpG-rich transcription start sites of active genes, thereby promoting histone H2B GlcNAcylation by OGT.

As used herein, “TET2” refers to “ten-eleven translocation 2 (TET2),” a member of the TET family of enzymes, encoded by the TET1 gene. Similarly to TET1, TET2 is a dioxygenase that catalyzes the conversion of the modified genomic base 5-methylcytosine (5mC) into 5-hydroxymethylcytosine (ShmC) and plays a key role in active DNA demethylation. TET2 a preference for 5-hydroxymethylcytosine in CpG motifs. TET2 also mediates subsequent conversion of ShmC into 5-formylcytosine (5fC), and conversion of 5fC to 5-carboxylcytosine (ScaC). The conversion of 5mC into ShmC, 5fC and ScaC probably constitutes the first step in cytosine demethylation. Methylation at the C5 position of cytosine bases is an epigenetic modification of the mammalian genome which plays an important role in transcriptional regulation. In addition to its role in DNA demethylation, also involved in the recruitment of the O-GlcNAc transferase OGT to CpG-rich transcription start sites of active genes, thereby promoting histone H2B GlcNAcylation by OGT.

As used herein “DNMT3a-3L” refers to a fusion of a DNA methyltransferase, Dnmt3a and a Dnmt3L which is catalytically inactive, but directly interacts with the catalytic domains of Dnmt3a.

In some embodiments, a suitable epigenetic recruiter for use in the agent, compositions, and methods of the invention comprises a MQ1 domain, a DNMT3a-3L domain, a TET1 domain, or a TET2 domain. In one embodiment, a suitable epigenetic recruiter for use in the agent, compositions, and methods of the invention comprises a dCas9-MQ1 fusion, a dCas9-DNMT3a-3L fusion, a dCas9-TET1 fusion or a dCAS9-TET2 fusion.

### Antibodies or Antigen Binding Fragments Thereof

The agents used in the methods of the present invention further contemplate anti-Yap antibodies or antigen binding fragments thereof, thereby inhibiting the expression and/or activity of Yap in a cell, and reducing heterotopic ossification in the cell. In one embodiment, the anti-Yap antibody, or antigen binding fragment thereof decreases Yap rRNA expression and/or Yap protein expression. In another embodiment, the anti-Yap antibody, or antigen binding fragment thereof, inhibits the activity of Yap, e.g., blocking the transcriptional activation of target genes.

In another aspect, the invention also contemplates methods and compositions comprising an antibody which binds to a protein that interacts with Yap, e.g., Shh or CDK7, thereby inhibiting the expression and/or activity of the interacting protein, e.g., Shh or CKD7, in a cell, and reducing heterotopic ossification in the cell. In one embodiment, the anti-Shia antibody, or antigen binding fragment thereof, decreases Shh mRNA expression and/or Shh protein expression. In another embodiment, the anti-Shh antibody, or antigen binding fragment thereof, inhibits the activity of Shh, e.g., potentiation of Yap expression.

The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. An “antibody” refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from N terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., Yap). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3rd ed. 1993); (iv) a Fd fragment consisting of the VH and CH1 domains; (v) a Fv fragment consisting of the VL and VH domains of a single arm of an antibody, (vi) a dAb fragment (Ward et al. (1989) Nature 341: 544-546), which consists of a VH domain; (vii) an isolated complementarity determining region (CDR); and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An “isolated antibody”, as used herein, is intended to refer to an antibody that is substantially free of other antibodies having different antigenic specificities (e.g., an isolated antibody that specifically binds to, e.g., Yap, is substantially free of antibodies that specifically bind antigens other than Yap). Moreover, an isolated antibody may be substantially free of other cellular material and/or chemicals. An “isolated antibody” may, however, include polyclonal antibodies, which all bind specifically to, e.g., Yap.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity, which have variable regions in which both the framework and CDR regions are derived from human germline immunoglobulin sequences. In one embodiment, the human monoclonal antibodies are produced by a hybridoma, which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences. It will be appreciated by one of skill in the art that when a sequence is “derived” from a particular species, said sequence may be a protein sequence, such as when variable region amino acids are taken from a murine antibody, or said sequence may be a DNA sequence, such as when variable region encoding nucleic acids are taken from murine DNA. A humanized antibody may also be designed based on the known sequences of human and non-human (e.g., murine or rabbit) antibodies. The designed antibodies, potentially incorporating both human and non-human residues, may be chemically synthesized. The sequences may also be synthesized at the DNA level and expressed in vitro or in vivo to generate the humanized antibodies.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “antibody mimetic” or “antibody mimic” is intended to refer to molecules capable of mimicking an antibody's ability to bind an antigen, but which are not limited to native antibody structures. Examples of such antibody mimetics include, but are not limited to, Adnectins (i.e., fibronectin based binding molecules), Affibodies, DARPins, Anticalins, Avimers, and Versabodies all of which employ binding structures that, while they mimic traditional antibody binding, are generated from and function via distinct mechanisms. The embodiments of the instant invention, as they are directed to antibodies, or antigen-binding portions thereof, also apply to the antibody mimetics described above.

As used herein, an antibody that “specifically binds” to an antigen, e.g., Yap, is intended to refer to an antibody that binds to the antigen with a K_{D} of 1×10⁻⁷ M or less, more preferably 5×10⁻⁸M or less, more preferably 1×10⁻⁸ M or less, more preferably 5×10⁻⁹ M or less.

The term “does not substantially bind” to a protein or cells, as used herein, means does not bind or does not bind with a high affinity to the protein or cells, i.e., binds to the protein or cells with a K_{D} of 1×10⁻⁶ M or more, more preferably 1×10⁻⁵M or more, more preferably 1×10⁻⁴M or more, more preferably 1×10⁻³ M or more, even more preferably 1×10⁻² M or more.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore® system.

As used herein, the term “high affinity”, when referring an IgG type antibody, refers to an antibody having a K_{D} of 10⁻⁸ M or less, more preferably 10⁻⁹ M or less and even more preferably 10⁻¹⁰ M or less for, e.g., Yap. However, “high affinity” binding can vary for other antibody isotypes. For example, “high affinity” binding for an IgM isotype refers to an antibody having a K_{D} of 10⁻⁷M or less, more preferably 10⁻⁸M or less, even more preferably 10⁻⁹ M or less.

Preferrably, the antibody binds to Yap with a K_{D} of 5×10⁻⁸M or less, a K_{D} of 1×10⁻⁸M or less, a K_{D} of 5×10⁻⁹ M or less, or a K_{D} of between 1×10⁻⁸M and 1×10⁻¹⁰ M or less. Standard assays to evaluate the binding ability of the antibodies toward Yap are known in the art, including for example, ELISAs, Western blots and RIAs. The binding kinetics (e.g., binding affinity) of the antibodies also can be assessed by standard assays known in the art, such as by ELISA, Scatchard and Biacore analysis.

### Engineered and Modified Antibodies

The V_{H} and/or V_{L} sequences of an antibody prepared according the methods of the present invention and may be used as starting material to engineer a modified antibody, which modified antibody may have altered properties from the starting antibody. An antibody can be engineered by modifying one or more residues within one or both of the original variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al. (1998) Nature 332: 323-327; Jones et al. (1986) Nature 321: 522-525; Queen et al. (1989) Proc. Natl. Acad. Sci. U.S.A. 86: 10029-10033; U.S. Pat. No. 5,225,539 to Winter, and U.S. Pat. Nos. 5,530,101; 5,585,089; 5,693,762 and 6,180,370 to Queen et al.)

Framework sequences for antibodies can be obtained from public DNA databases or published references that include germline antibody gene sequences. For example, germline DNA sequences for human heavy and light chain variable region genes can be found in the “VBase” human germline sequence database (available on the Internet at mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson et al. (1992) J. Mol. Biol. 227: 776-798; and Cox et al. (1994) Eur. J. Immunol. 24: 827-836; the contents of each of which are expressly incorporated herein by reference. As another example, the germline DNA sequences for human heavy and light chain variable region genes can be found in the Genbank database.

Antibody protein sequences are compared against a compiled protein sequence database using one of the sequence similarity searching methods called the Gapped BLAST (Altschul et al. (1997) Nucleic Acids Res. 25: 3389-3402), which is well known to those skilled in the art. BLAST is a heuristic algorithm in that a statistically significant alignment between the antibody sequence and the database sequence is likely to contain high-scoring segment pairs (HSP) of aligned words. Segment pairs whose scores cannot be improved by extension or trimming is called a hit. Briefly, the nucleotide sequences of VBASE origin (vbase.mrc-cpe.cam.ac.uk/vbase1/list2.php) are translated and the region between and including FR1 through FR3 framework region is retained. The database sequences have an average length of 98 residues. Duplicate sequences, which are exact matches over the entire length of the protein, are removed. A BLAST search for proteins using the program blastp with default, standard parameters except the low complexity filter, which is turned off, and the substitution matrix of BLOSUM62, filters for the top 5 hits yielding sequence matches. The nucleotide sequences are translated in all six frames and the frame with no stop codons in the matching segment of the database sequence is considered the potential hit. This is in turn confirmed using the BLAST program tblastx, which translates the antibody sequence in all six frames and compares those translations to the VBASE nucleotide sequences dynamically translated in all six frames. Other human germline sequence databases, such as that available from IMGT (http://imgt.cines.fr), can be searched similarly to VBASE as described above.

The identities are exact amino acid matches between the antibody sequence and the protein database over the entire length of the sequence. The positives (identities+substitution match) are not identical but amino acid substitutions guided by the BLOSUM62 substitution matrix. If the antibody sequence matches two of the database sequences with same identity, the hit with most positives would be decided to be the matching sequence hit.

Identified V_{H} CDR1, CDR2, and CDR3 sequences, and the V_{K} CDR1, CDR2, and CDR3 sequences, can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen-binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays known in the art. For example, an antibody of the present invention may be mutated to create a library, which may then be screened for binding to an antigen, e.g., Yap. Preferably conservative modifications (as discussed above) are introduced. The mutations may be amino acid substitutions, additions or deletions, but are preferably substitutions. Moreover, typically no more than one, two, three, four or five residues within a CDR region are altered.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in futher detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. These strategies will be effective as long as the binding of the antibody to an antigen, e.g., Yap, is not compromised.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγR1, FcγRII, FcγRIII and FcRn have been mapped and variants with improved binding have been described (see Shields et al. (2001) J. Biol. Chem. 276: 6591-6604). Specific mutations at positions 256, 290, 298, 333, 334 and 339 were shown to improve binding to FcγRIII Additionally, the following combination mutants were shown to improve FcγRIII binding: T256A/S298A, S298A/E333A, S298A/K224A and S298A/E333A/K334A.

In still another embodiment, the C-terminal end of an antibody of the present invention is modified by the introduction of a cysteine residue as is described in U.S. Provisional Application Ser. No. 60/957,271, which is hereby incorporated by reference in its entirety. Such modifications include, but are not limited to, the replacement of an existing amino acid residue at or near the C terminus of a full-length heavy chain sequence, as well as the introduction of a cysteine-containing extension to the C terminus of a full-length heavy chain sequence. In preferred embodiments, the cysteine-containing extension comprises the sequence alanine-alanine-cysteine (from N-terminal to C-terminal).

In preferred embodiments the presence of such C-terminal cysteine modifications provide a location for conjugation of a partner molecule, such as a therapeutic agent or a marker molecule. In particular, the presence of a reactive thiol group, due to the C-terminal cysteine modification, can be used to conjugate a partner molecule employing the disulfide linkers described in detail below. Conjugation of the antibody to a partner molecule in this manner allows for increased control over the specific site of attachment. Furthermore, by introducing the site of attachment at or near the C terminus, conjugation can be optimized such that it reduces or eliminates interference with the antibody's functional properties, and allows for simplified analysis and quality control of conjugate preparations.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 to Co et al. Additional approaches for altering glycosylation are described in further detail in U.S. Pat. No. 7,214,775 to Hanai et al., U.S. Pat. No. 6,737,056 to Presta, U.S. Pub No. 20070020260 to Presta, PCT Publication No. WO/2007/084926 to Dickey et al., PCT Publication No. WO/2006/089294 to Zhu et al., and PCT Publication No. WO/2007/055916 to Ravetch et al., each of which is hereby incorporated by reference in its entirety.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol. Bioeng. 87: 614-622). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields et al. (2002) J. Biol. Chem. 277: 26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17: 176-180). Alternatively, the fucose residues of the antibody may be cleaved off using a fucosidase enzyme. For example, the fucosidase alpha-L-fucosidase removes fucosyl residues from antibodies (Tarentino et al. (1975) Biochem. 14: 5516-5523).

Additionally or alternatively, an antibody can be made that has an altered type of glycosylation, wherein that alteration relates to the level of sialyation of the antibody. Such alterations are described in PCT Publication No. WO/2007/084926 to Dickey et al., and PCT Publication No. WO/2007/055916 to Ravetch et al., both of which are incoporated by reference in their entirety. For example, one may employ an enzymatic reaction with sialidase, such as, for example, Arthrobacter ureafacens sialidase. The conditions of such a reaction are generally described in the U.S. Pat. No. 5,831,077, which is hereby incorporated by reference in its entirety. Other non-limiting examples of suitable enzymes are neuraminidase and N-Glycosidase F, as described in Schloemer et al. (1975) J. Virol. 15, 882-893 and in Leibiger et al. (1999) Biochem. J. 338, 529-538, respectively. Desialylated antibodies may be further purified by using affinity chromatography. Alternatively, one may employ methods to increase the level of sialyation, such as by employing sialytransferase enzymes. Conditions of such a reaction are generally described in Basset et al. (2000) Scand. J. Immunol. 51: 307-311.

Another modification of the antibodies herein that is contemplated by the invention is pegylation. An antibody can be pegylated to, for example, increase the biological (e.g., serum) half-life of the antibody. To pegylate an antibody, the antibody, or antigen-binding fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al. As such, the methods of pegylation described here also apply to the peptidic molecules of the invention described below.

### Antibody Fragments and Antibody Mimetics

The instant invention is not limited to traditional antibodies and may be practiced through the use of antibody fragments and antibody mimetics. As detailed below, a wide variety of antibody fragment and antibody mimetic technologies have now been developed and are widely known in the art. While a number of these technologies, such as domain antibodies, Nanobodies, and UniBodies make use of fragments of, or other modifications to, traditional antibody structures, there are also alternative technologies, such as Adnectins, Affibodies, DARPins, Anticalins, Avimers, and Versabodies that employ binding structures that, while they mimic traditional antibody binding, are generated from and function via distinct mechanisms. Some of these alternative structures are reviewed in Gill and Damle (2006) Curr Opinion Biotechnol 17: 653-658.

Domain Antibodies (dAbs) are the smallest functional binding units of antibodies, corresponding to the variable regions of either the heavy (VH) or light (VL) chains of human antibodies. Domain Antibodies have a molecular weight of approximately 13 kDa. Domantis has developed a series of large and highly functional libraries of fully human VH and VL dAbs (more than ten billion different sequences in each library), and uses these libraries to select dAbs that are specific to therapeutic targets. In contrast to many conventional antibodies, domain antibodies are well expressed in bacterial, yeast, and mammalian cell systems. Further details of Domain Antibodies and methods of production thereof may be obtained by reference to U.S. Pat. Nos. 6,291,158; 6,582,915; 6,593,081; 6,172,197; 6,696,245; U.S. Serial No. 2004/0110941; European patent application No. 1433846 and European Patents 0368684 & 0616640; WO05/035572, WO04/101790, WO04/081026, WO04/058821, WO04/003019 and WO03/002609, each of which is herein incorporated by reference in its entirety.

Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a perfectly stable polypeptide harboring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see, e.g., WO 04/041867, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique three-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The Nanoclone method (see, e.g., WO 06/079372, which is herein incorporated by reference in its entirety) is a proprietary method for generating Nanobodies against a desired target, based on automated high-throughout selection of B-cells and could be used in the context of the instant invention.

UniBodies are another antibody fragment technology; however, this one is based upon the removal of the hinge region of IgG4 antibodies. The deletion of the hinge region results in a molecule that is essentially half the size of traditional IgG4 antibodies and has a univalent binding region rather than the bivalent binding region of IgG4 antibodies. It is also well known that IgG4 antibodies are inert and thus do not interact with the immune system, which may be advantageous for the treatment of diseases where an immune response is not desired, and this advantage is passed onto UniBodies. For example, UniBodies may function to inhibit or silence, but not kill, the cells to which they are bound. Additionally, UniBody binding to cancer cells do not stimulate them to proliferate. Furthermore, because UniBodies are about half the size of traditional IgG4 antibodies, they may show better distribution over larger solid tumors with potentially advantageous efficacy. UniBodies are cleared from the body at a similar rate to whole IgG4 antibodies and are able to bind with a similar affinity for their antigens as whole antibodies. Further details of UniBodies may be obtained by reference to patent application WO2007/059782, which is herein incorporated by reference in its entirety.

Adnectin molecules are engineered binding proteins derived from one or more domains of the fibronectin protein. Fibronectin exists naturally in the human body. It is present in the extracellular matrix as an insoluble glycoprotein dimer and also serves as a linker protein. It is also present in soluable form in blood plasma as a disulphide linked dimer. The plasma form of fibronectin is synthesized by liver cells (hepatocytes), and the ECM form is made by chondrocytes, macrophages, endothelial cells, fibroblasts, and some cells of the epithelium (see Ward M., and Marcey, D., callutheran.edu/AcademicPrograms/DepartmentsBioDev/omm/fibro/fibro.htm). As mentioned previously, fibronectin may function naturally as a cell adhesion molecule, or it may mediate the interaction of cells by making contacts in the extracellular matrix. Typically, fibronectin is made of three different protein modules, type I, type II, and type III modules. For a review of the structure of function of the fibronectin, see Pankov and Yamada (2002) J. Cell Sci. 115: 3861-3863, Hohenester and Engel (2002) Journal? 21: 115-128, and Lucena et al. (2007) Invest. Clin. 48: 249-262.

In a preferred embodiment, adnectin molecules are derived from the fibronectin type III domain by altering the native protein, which is composed of multiple beta strands distributed between two beta sheets. Depending on the originating tissue, fibronecting may contain multiple type III domains, which may be denoted, e.g., ¹Fn3, ²Fn3, ³Fn3, etc. The ¹⁰Fn3 domain contains an integrin-binding motif and further contains three loops that connect the beta strands. These loops may be thought of as corresponding to the antigen-binding loops of the IgG heavy chain, and they may be altered by methods discussed below to specifically bind a target of interest, e.g., Yao. Preferably, a fibronectin type III domain useful for the purposes of this invention is a sequence which exhibits a sequence identity of at least 30%, at least 40%, at least 50%, at least 60%, at least 70%, at least 80%, at least 90%, or at least 95% to the sequence encoding the structure of the fibronectin type III molecule which can be accessed from the Protein Data Bank (PDB, rcsb.org/pdb/home/home.do) with the accession code: lttg. Adnectin molecules may also be derived from polymers of ¹⁰Fn3 related molecules rather than a simple monomeric ¹⁰Fn3 structure.

Although the native ¹⁰Fn3 domain typically binds to integrin, ¹⁰Fn3 proteins adapted to become adnectin molecules are altered so to bind antigens of interest, e.g., Yap. In one embodiment, the alteration to the ¹⁰Fn3 molecule comprises at least one mutation to a beta strand.

The alterations in the ¹⁰Fn3 may be made by any method known in the art including, but not limited to, error prone PCR, site-directed mutagenesis, DNA shuffling, or other types of recombinational mutagenesis, which have been referenced herein. In one example, variants of the DNA encoding the ¹⁰Fn3 sequence may be directly synthesized in vitro, and later transcribed and translated in vitro or in vivo. Alternatively, a natural ¹⁰Fn3 sequence may be isolated or cloned from the genome using standard methods (as performed, e.g., in U.S. Pat. Application No. 20070082365), and then mutated using mutagenesis methods known in the art.

In one embodiment, a target protein, e.g., Yap, may be immobilized on a solid support, such as a column resin or a well in a microtiter plate. The target is then contacted with a library of potential binding proteins. The library may comprise ¹⁰Fn3 clones or adnectin molecules derived from the wild type ¹⁰Fn3 by mutagenesis/randomization of the ¹⁰Fn3 sequence or by mutagenesis/randomization of the ¹⁰Fn3 loop regions (not the beta strands). In a preferred embodiment the library may be an RNA-protein fusion library generated by the techniques described in Szostak et al., U.S. Ser. Nos. 09/007,005 and 09/247,190; Szostak et al., WO989/31700; and Roberts & Szostak (1997) 94:12297-12302. The library may also be a DNA-protein library (e.g., as described in Lohse, U.S. Ser. No. 60/110,549, U.S. Ser. No. 09/459,190, and WO 00/32823). The fusion library is then incubated with the immobilized target (e.g., Yap) and the solid support is washed to remove non-specific binding moieties. Tight binders are then eluted under stringent conditions and PCR is used to amply the genetic information or to create a new library of binding molecules to repeat the process (with or without additional mutagenesis). The selection/mutagenesis process may be repeated until binders with sufficient affinity to the target are obtained. Adnectin molecules for use in the present invention may be engineered using the PROfusion™ technology employed by Adnexus, a Briston-Myers Squibb company. The PROfusion technology was created based on the techniques referenced above (e.g., Roberts and Szostak (1997) Proc. Natl. Acad. Sci. USA 94: 12297-12302). Methods of generating libraries of altered ¹⁰Fn3 domains and selecting appropriate binders which may be used with the present invention are described fully in the following U.S. Patent and Patent Application documents and are incorporated herein by reference: U.S. Pat. Nos. 7,115,396; 6,818,418; 6,537,749; 6,660,473; 7,195,880; 6,416,950; 6,214,553; 6,623,926; 6,312,927; 6,602,685; 6,518,018; 6,207,446; 6,258,558; 6,436,665; 6,281,344; 7,270,950; 6,951,725; 6,846,655; 7,078,197; 6,429,300; 7,125,669; 6,537,749; 6,660,473; and U.S. Pat. Application Nos. 20070082365; 20050255548; 20050038229; 20030143616; 20020182597; 20020177158; 20040086980; 20040253612; 20030022236; 20030013160; 20030027194; 20030013110; 20040259155; 20020182687; 20060270604; 20060246059; 20030100004; 20030143616; and 20020182597. The generation of diversity in fibronectin type III domains, such as ¹⁰Fn3, followed by a selection step may be accomplished using other methods known in the art such as phage display, ribosome display, or yeast surface display, e.g., Lipovsĕk et al. (2007) J. Mol. Biol. 368: 1024-1041; Sergeeva et al. (2006) Adv. Drug Deliv. Rev. 58: 1622-1654; Petty et al. (2007) Trends Biotechnol. 25: 7-15; Rothe et al. (2006) Expert Opin. Biol. Ther. 6: 177-187; and Hoogenboom (2005) Nat. Biotechnol. 23: 1105-1116.

It should be appreciated by one of skill in the art that the methods references cited above may be used to derive antibody mimics from proteins other than the preferred ¹⁰Fn3 domain. Additional molecules that can be used to generate antibody mimics via the above referenced methods include, without limitation, human fibronectin modules ¹Fn3-⁹Fn3 and ¹¹Fn3-¹⁷Fn3 as well as related Fn3 modules from non-human animals and prokaryotes. In addition, Fn3 modules from other proteins with sequence homology to ¹⁰Fn3, such as tenascins and undulins, may also be used. Other exemplary proteins having immunoglobulin-like folds (but with sequences that are unrelated to the V_{H} domain) include N-cadherin, ICAM-2, titin, GCSF receptor, cytokine receptor, glycosidase inhibitor, E-cadherin, and antibiotic chromoprotein. Further domains with related structures may be derived from myelin membrane adhesion molecule P0, CD8, CD4, CD2, class I MHC, T-cell antigen receptor, CD1, C2 and I-set domains of VCAM-1, I-set immunoglobulin fold of myosin-binding protein C, I-set immunoglobulin fold of myosin-binding protein H, I-set immunoglobulin-fold of telokin, telikin, NCAM, twitchin, neuroglian, growth hormone receptor, erythropoietin receptor, prolactin receptor, GC-SF receptor, interferon-gamma receptor, beta-galactosidase/glucuronidase, beta-glucuronidase, and transglutaminase. Alternatively, any other protein that includes one or more immunoglobulin-like folds may be utilized to create an adnecting-like binding moiety. Such proteins may be identified, for example, using the program SCOP (Murzin et al. (1995) J. Mol. Biol. 247: 536; Lo Conte et al. (2000) Nucleic Acids Res. 25: 257.

An aptamer is another type of antibody-mimetic that is encompassed by the present invention. Aptamers are typically small nucleotide polymers that bind to specific molecular targets. Aptamers may be single- or double-stranded nucleic acid molecules (DNA or RNA), although DNA based aptamers are most commonly double-stranded. There is no defined length for an aptamer nucleic acid; however, aptamer molecules are most commonly between 15 and 40 nucleotides long.

Aptamers often form complex three-dimensional structures, which determine their affinity for target molecules. Aptamers can offer many advantages over simple antibodies, primarily because they can be engineered and amplified almost entirely in vitro. Furthermore, aptamers often induce little or no immune response.

Aptamers may be generated using a variety of techniques, but were originally developed using in vitro selection (Ellington and Szostak (1990) Nature 346: 818-822) and the SELEX method (systematic evolution of ligands by exponential enrichment) (Schneider et al. (1992) J. Mol. Biol. 228: 862-869) the contents of which are incorporated herein by reference. Other methods to make and uses of aptamers have been published including Klussmann. The Aptamer Handbook: Functional Oligonucleotides and Their Applications. ISBN: 978-3-527-31059-3; Ulrich et al. (2006) Comb. Chem. High Throughput Screen 9: 619-632; Cerchia and de Franciscis (2007) Methods Mol. Biol. 361: 187-200; Ireson and Kelland (2006) Mol. Cancer Ther. 5: 2957-2962; U.S. Pat. Nos. 5,582,981; 5,840,867; 5,756,291; 6,261,783; 6,458,559; 5,792,613; 6,111,095; and U.S. patent application Ser. Nos. 11/482,671; 11/102,428; 11/291,610; and Ser. No. 10/627,543, which are all incorporated herein by reference.

The SELEX method is clearly the most popular and is conducted in three fundamental steps. First, a library of candidate nucleic acid molecules is selected from for binding to specific molecular target. Second, nucleic acids with sufficient affinity for the target are separated from non-binders. Third, the bound nucleic acids are amplified, a second library is formed, and the process is repeated. At each repetition, aptamers are chosen that have higher and higher affinity for the target molecule. SELEX methods are described more fully in the following publications, which are incorporated herein by reference: Bugaut et al. (2006) 4: 4082-4088; Stoltenburg et al. (2007) Biomol. Eng. 24: 381-403; and Gopinath (2007) Anal. Bioanal. Chem. 387: 171-182.

An “aptamer” of the invention also includes aptamer molecules made from peptides instead of nucleotides. Peptide aptamers share many properties with nucleotide aptamers (e.g., small size and ability to bind target molecules with high affinity) and they may be generated by selection methods that have similar principles to those used to generate nucleotide aptamers, for example Baines and Colas (2006) Drug Discov. Today 11: 334-341; and Bickle et al. (2006) Nat. Protoc. 1: 1066-1091, which are incorporated herein by reference.

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord et al. (1997) Nat. Biotechnol. 15: 772-777; Ronmark et al. (2002) Eur. J. Biochem. 269: 2647-2655). The simple, robust structure of Affibody molecules in combination with their low molecular weight (6 kDa), make them suitable for a wide variety of applications, for instance, as detection reagents (Ronmark et al. (2002) J. Immunol. Methods 261: 199-211) and to inhibit receptor interactions (Sandstorm et al. (2003) Protein Eng. 16: 691-697). Further details of Affibodies and methods of production thereof may be obtained by reference to U.S. Pat. No. 5,831,012, which is herein incorporated by reference in its entirety.

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains.

DARPins can be produced in bacterial expression systems at very high yields and they belong to the most stable proteins known. Highly specific, high-affinity DARPins to a broad range of target proteins, including human receptors, cytokines, kinases, human proteases, viruses and membrane proteins, have been selected. DARPins having affinities in the single-digit nanomolar to picomolar range can be obtained.

DARPins have been used in a wide range of applications, including ELISA, sandwich ELISA, flow cytometric analysis (FACS), immunohistochemistry (IHC), chip applications, affinity purification or Western blotting. DARPins also proved to be highly active in the intracellular compartment, for example, as intracellular marker proteins fused to green fluorescent protein (GFP). DARPins were further used to inhibit viral entry with IC50 in the pM range. DARPins are not only ideal to block protein-protein interactions, but also to inhibit enzymes. Proteases, kinases and transporters have been successfully inhibited, most often an allosteric inhibition mode. Very fast and specific enrichments on the tumor and very favorable tumor to blood ratios make DARPins well suited for in vivo diagnostics or therapeutic approaches.

Additional information regarding DARPins and other DRP technologies can be found in U.S. Patent Application Publication No. 2004/0132028 and International Patent Application Publication No. WO 02/20565, both of which are hereby incorporated by reference in their entirety.

Anticalins are an additional antibody mimetic technology; however, in this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved ß-barrel that supports four loops at one terminus of the protein. These loops form the entrance to a binding pocket and conformational differences in this part of the molecule account for the variation in binding specificity between individual lipocalins.

While the overall structure of hypervariable loops supported by a conserved ß-sheet framework is reminiscent of immunoglobulins, lipocalins differ considerably from antibodies in terms of size, being composed of a single polypeptide chain of 160-180 amino acids, which is marginally larger than a single immunoglobulin domain.

Lipocalins are cloned and their loops are subjected to engineering in order to create Anticalins. Libraries of structurally diverse Anticalins have been generated and Anticalin display allows the selection and screening of binding function, followed by the expression and production of soluble protein for further analysis in prokaryotic or eukaryotic systems. Studies have successfully demonstrated that Anticalins can be developed that are specific for virtually any human target protein can be isolated and binding affinities in the nanomolar or higher range can be obtained (see, e.g., Schlehuber and Skerna (2005) Drug Dis Today 10:23).

Anticalins can also be formatted as dual targeting proteins, so-called Duocalins. A Duocalin binds two separate therapeutic targets in one easily produced monomeric protein using standard manufacturing processes while retaining target specificity and affinity regardless of the structural orientation of its two binding domains.

Modulation of multiple targets through a single molecule is particularly advantageous in diseases known to involve more than a single causative factor. Moreover, bi- or multivalent binding formats such as Duocalins have significant potential in targeting cell surface molecules in disease, mediating agonistic effects on signal transduction pathways or inducing enhanced internalization effects via binding and clustering of cell surface receptors. Furthermore, the high intrinsic stability of Duocalins is comparable to monomeric Anticalins, offering flexible formulation and delivery potential for Duocalins.

Additional information regarding Anticalins can be found in U.S. Pat. No. 7,250,297 and International Patent Application Publication No. WO 99/16873, both of which are hereby incorporated by reference in their entirety.

Another antibody mimetic technology useful in the context of the instant invention are Avimers. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in E. coli, improved thermostability and resistance to proteases. Avimers with sub-nanomolar affinities have been obtained against a variety of targets.

Additional information regarding Avimers can be found in U.S. Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Versabodies are another antibody mimetic technology that could be used in the context of the instant invention. Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well known to affect immunogenicity, and together they are expected to cause a large decrease in immunogenicity.

The inspiration for Versabodies comes from the natural injectable biopharmaceuticals produced by leeches, snakes, spiders, scorpions, snails, and anemones, which are known to exhibit unexpectedly low immunogenicity. Starting with selected natural protein families, by design and by screening the size, hydrophobicity, proteolytic antigen processing, and epitope density are minimized to levels far below the average for natural injectable proteins.

Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable (they can be boiled) and offer extended shelf-life.

Additional information regarding Versabodies can be found in U.S. Patent Application Publication No. 2007/0191272, which is hereby incorporated by reference in its entirety.

SMIPs™ (Small Modular ImmunoPharmaceuticals-Trubion Pharmaceuticals) have been engineered to maintain and optimize target binding, effector functions, in vivo half-life, and expression levels. SMIPS consist of three distinct modular domains. First, they contain a binding domain that may consist of any protein that confers specificity (e.g., cell surface receptors, single chain antibodies, soluble proteins, etc). Secondly, they contain a hinge domain, which serves as a flexible linker between the binding domain and the effector domain, and also helps control multimerization of the SMIP drug. Finally, SMIPS contain an effector domain, which may be derived from a variety of molecules including Fc domains or other specially designed proteins. The modularity of the design, which allows the simple construction of SMIPs with a variety of different binding, hinge, and effector domains, provides for rapid and customizable drug design.

More information on SMIPs, including examples of how to design them, may be found in Zhao et al. (2007) Blood 110: 2569-2577 and the following U.S. Pat. App. Nos. 20050238646; 20050202534; 20050202028; 20050202023; 20050202012; 20050186216; 20050180970; and 20050175614.

The detailed description of antibody fragment and antibody mimetic technologies provided above is not intended to be a comprehensive list of all technologies that could be used in the context of the instant specification. For example, and also not by way of limitation, a variety of additional technologies including alternative polypeptide-based technologies, such as fusions of complimentary determining regions as outlined in Qui et al. (2007) Nat. Biotechnol. 25: 921-929, which is hereby incorporated by reference in its entirety, as well as nucleic acid-based technologies, such as the RNA aptamer technologies described in U.S. Pat. Nos. 5,789,157, 5,864,026, 5,712,375, 5,763,566, 6,013,443, 6,376,474, 6,613,526, 6,114,120, 6,261,774, and 6,387,620, all of which are hereby incorporated by reference, could be used in the context of the instant invention.

### Production of Antibodies of the Invention

Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)). Monoclonal and polyclonal antibodies to molecules, e.g., proteins, and markers also commercially available (R and D Systems, Minneapolis, Minn.; HyTest, HyTest Ltd., Turku Finland; Abcam Inc., Cambridge, Mass., USA, Life Diagnostics, Inc., West Chester, Pa., USA; Fitzgerald Industries International, Inc., Concord, Mass. 01742-3049 USA; BiosPacific, Emeryville, Calif.).

In some embodiments, the antibody is a polyclonal antibody. In other embodiments, the antibody is a monoclonal antibody.

Polyclonal antibodies of the present invention can be produced by a variety of techniques that are well known in the art. Polyclonal antibodies are derived from different B-cell lines and thus may recognize multiple epitopes on the same antigen. Polyclonal antibodies are typically produced by immunization of a suitable mammal with the antigen of interest, e.g., Yap. Animals often used for production of polyclonal antibodies are chickens, goats, guinea pigs, hamsters, horses, mice, rats, sheep, and, most commonly, rabbit. Standard methods to produce polyclonal antibodies are widely known in the art and can be combined with the methods of the present invention (e.g., U.S. Pat. Nos. 4,719,290, 6,335,163, 5,789,208, 2,520,076, 2,543,215, and 3,597,409, the entire contents of which are incorporated herein by reference.

Monoclonal antibodies of the present invention can be produced by any of a variety of techniques known to those of ordinary skill in the art (see, for example, Harlow and Lane, Antibodies: A Laboratory Manual, Cold Spring Harbor Laboratory, 1988). In general, antibodies can be produced by cell culture techniques, including the generation of monoclonal antibodies as described herein, or via transfection of antibody genes into suitable bacterial or mammalian cell hosts, in order to allow for the production of recombinant antibodies.

Monoclonal antibodies may be prepared using hybridoma methods, such as the technique of Kohler and Milstein (Eur. J. Immunol. 6:511-519, 1976), and improvements thereto. These methods involve the preparation of immortal cell lines capable of producing antibodies having the desired specificity. Monoclonal antibodies may also be made by recombinant DNA methods, such as those described in U.S. Pat. No. 4,816,567. DNA encoding antibodies employed in the disclosed methods may be isolated and sequenced using conventional procedures. Recombinant antibodies, antibody fragments, and/or fusions thereof, can be expressed in vitro or in prokaryotic cells (e.g. bacteria) or eukaryotic cells (e.g. yeast, insect or mammalian cells) and further purified as necessary using well known methods.

More particularly, monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner effective to stimulate antibody producing cells. The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals. Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

The selected hybridomas would then be serially diluted and cloned into individual antibody-producing cell lines, which clones may then be propagated indefinitely to provide MAbs. The cell lines may be exploited for MAb production in two basic ways. A sample of the hybridoma may be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, may then be tapped to provide MAbs in high concentration. The individual cell lines also may be cultured in vitro, where the MAbs are naturally secreted into the culture medium from which they may be readily obtained in high concentrations. MAbs produced by either means may be further purified, if desired, using filtration, centrifugation and various chromatographic methods such as HPLC or affinity chromatography.

Large amounts of the monoclonal antibodies of the present invention also may be obtained by multiplying hybridoma cells in vivo. Cell clones are injected into mammals which are histocompatible with the parent cells, e.g., syngeneic mice, to cause growth of antibody-producing tumors. Optionally, the animals are primed with a hydrocarbon, especially oils such as pristane (tetramethylpentadecane) prior to injection.

In accordance with the present invention, fragments of the monoclonal antibody of the invention may be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention may be synthesized using an automated peptide synthesizer.

Antibodies may also be derived from a recombinant antibody library that is based on amino acid sequences that have been designed in silico and encoded by polynucleotides that are synthetically generated. Methods for designing and obtaining in silico-created sequences are known in the art (Knappik et al., J. Mol. Biol. 296:254:57-86, 2000; Krebs et al., J. Immunol. Methods 254:67-84, 2001; U.S. Pat. No. 6,300,064).

Digestion of antibodies to produce antigen-binding fragments thereof can be performed using techniques well known in the art. For example, the proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′).sub.2” fragment, which comprises both antigen-binding sites. “Fv” fragments can be produced by preferential proteolytic cleavage of an IgM, IgG or IgA immunoglobulin molecule, but are more commonly derived using recombinant techniques known in the art. The Fv fragment includes a non-covalent V.sub.H::V.sub.L heterodimer including an antigen-binding site which retains much of the antigen recognition and binding capabilities of the native antibody molecule (Inbar et al., Proc. Natl. Acad. Sci. USA 69:2659-2662 (1972); Hochman et al., Biochem. 15:2706-2710 (1976); and Ehrlich et al., Biochem. 19:4091-4096 (1980)).

Antibody fragments that specifically bind to the protein biomarkers disclosed herein can also be isolated from a library of scFvs using known techniques, such as those described in U.S. Pat. No. 5,885,793.

A wide variety of expression systems are available in the art for the production of antibody fragments, including Fab fragments, scFv, VL and VHs. For example, expression systems of both prokaryotic and eukaryotic origin may be used for the large-scale production of antibody fragments. Particularly advantageous are expression systems that permit the secretion of large amounts of antibody fragments into the culture medium. Eukaryotic expression systems for large-scale production of antibody fragments and antibody fusion proteins have been described that are based on mammalian cells, insect cells, plants, transgenic animals, and lower eukaryotes. For example, the cost-effective, large-scale production of antibody fragments can be achieved in yeast fermentation systems. Large-scale fermentation of these organisms is well known in the art and is currently used for bulk production of several recombinant proteins.

Following screening and sequencing, antibodies may be produced using recombinant methods and compositions, e.g., as described in U.S. Pat. No. 4,816,567, incorporated by reference herein. An isolated nucleic acid encoding, for example, an anti-Yap antibody is used to transform host cells for expression. Such nucleic acid may encode an amino acid sequence comprising the VL and/or an amino acid sequence comprising the VII of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (I) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell).

For recombinant production of an anti-Yap antibody, a nucleic acid encoding an antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANIMODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney 011 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CVI); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals N.Y. Acad. Sci. 383:44-68 (1982); MRC 5 cells; and F54 cells. Other useful mammalian host cell lines include Chinese hamster ovary (CHO) cells, including DHFR.sup.-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Antibodies, or antigen binding fragments thereof, described herein are capable of binding to target proteins, such as Yap or Shh, thereby inhibiting heterotopic ossification in the cell. In some instances, antibodies, or antigen binding fragments thereof, described herein can inhibit the expression and/or activity of target proteins by at least 20%, e.g., 30%, 40%, 50%, 60%, 70%, 80%, 90%, 95%, or higher. In some instances, antibodies described herein can inhibit heterotopic ossification in a cell by at least 20%, e.g., 30%, 40%, 50%, 60%, 70%, 80%, 90%, 95%, or higher.

In one embodiment, the anti-Yap antibody, or antigen binding portion thereof, inhibits heterotopic ossification (HO) in a cell. In another embodiment, the anti-Shh antibody, or antigen binding portion thereof, inhibits heterotopic ossification (HO) in a cell.

### Small Molecules

In another aspect of the invention, the agent that inhibits the expression and/or activity of Yap is a small molecule.

The small molecules of the instant invention are characterized by particular functional features or properties. For example, the small molecules bind to Yap, or any other protein that interacts with Yap, e.g., Shh, CDK7, or any other components of the Hedgehog pathway, e.g., Dhh, Ihh, Patched (Ptch1, Ptch2), Gli1, Gli2, Gli3, and Smoothened (Smo). In preferred embodiments, the binding of small molecule inhibitors to Yap will prevent the transcriptional activation of target genes of Yap, thereby inhibiting the activity of Yap. In other words, the small molecule may bind to the interacting partner of Yap, e.g., Shh or CDK7, thereby preventing activation of Yap activity.

The terms “small molecule compounds”, “small molecule drugs”, “small molecules”, or “small molecule inhibitors” are used interchangeably herein to refer to the compounds of the present invention screened for an effect on Yap and their ability to inhibit the activity of Yap. These compounds may comprise compounds in PubChem database (pubchem.ncbi.nlm.nih.gov/), the Molecular Libraries Screening Center Network (MLSCN) database, compounds in related databases, or derivatives and/or functional analogues thereof.

As used herein, “analogue” or “functional analogue” refers to a chemical compound or small molecule inhibitor that is structurally similar to a parent compound, but differs slightly in composition (e.g., one or more atoms or functional groups are added, removed, or modified). The analogue may or may not have different chemical or physical properties than the original compound and may or may not have improved biological and/or chemical activity. For example, the analogue may be more hydrophobic or it may have altered activity (increased, decreased, or identical to parent compound) as compared to the parent compound. The analogue may be a naturally or non-naturally occurring (e.g., recombinant) variant of the original compound. Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, “derivative” refers to a chemically or biologically modified version of a chemical compound or small molecule inhibitor that is structurally similar to a parent compound and (actually or theoretically) derivable from that parent compound. A “derivative” differs from an “analogue” or “functional analogue” in that a parent compound may be the starting material to generate a “derivative,” whereas the parent compound may not necessarily be used as the starting material to generate an “analogue” or “functional analogue.” A derivative may or may not have different chemical or physical properties of the parent compound. For example, the derivative may be more hydrophilic or it may have altered reactivity as compared to the parent compound. Derivatization (i.e., modification by chemical or other means) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al. (1996) Adv. Drug Deliv. Rev. 19: 115; Design of Prodrugs, H. Bundgaard (ed.), Elsevier, 1985; and H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups such as carboxylic acid groups can form alkali metal salts or alkaline earth metal salts (e.g., sodium salts, potassium salts, magnesium salts, calcium salts, and salts with physiologically tolerable quaternary ammonium ions and acid addition salts with ammonia and physiologically tolerable organic amines such as triethylamine, ethanolamine, or tris-(2-hydroxyethyl)amine). Basic groups can form acid addition salts, for example with inorganic acids such as hydrochloric acid (“HCl”), sulfuric acid, or phosphoric acid, or with organic carboxylic acids and sulfonic acids such as acetic acid, citric acid, benzoic acid, maleic acid, fumaric acid, tartaric acid, methanesulfonic acid, or p-toluenesulfonic acid. Compounds that simultaneously contain a basic group and an acidic group such as a carboxyl group in addition to basic nitrogen atoms can be present as zwitterions. Salts can be obtained by customary methods known to those skilled in the art, for example, by combining a compound with an inorganic or organic acid or base in a solvent or diluent, or from other salts by cation exchange or anion exchange.

Small molecules are known to have molecular weights of 1200 or below, 1000 or below, 900 or below, 800 or below, 700 or below, 600 or below, 500 or below, 400 or below, 300 or below, 200 or below, 100 or below, 50 or below, 25 or below, or 10 or below.

The small molecules of the present invention are selected or designed to bind to Yap, or any other protein that interacts with Yap, for example, Shh, CDK7, or any other components of the Hedgehog pathway, e.g., Dhh, Ihh, Patched (Ptch1, Ptch2), Gli1, Gli2, Gli3, and Smoothened (Smo). In some embodiments, the small molecule inhibitors are selected or designed to thereby antagonize the ability of Yap to become active, e.g., act as a transcriptional activator to regulate expression of target genes.

In preferred embodiments, a small molecule of the invention binds to Yap, or any other protein that interacts with Yap, for example, Shh, CDK7, with high affinity, for example, with an affinity of a K_{D} of 1×10⁻⁷ M or less, a K_{D} of 5×10⁻⁸M or less, a K_{D} of 1×10⁻⁸M or less, a K_{D} of 5×10⁻⁹ M or less, or a K_{D} of between 1×10⁻⁸M and 1×10⁻¹⁰ M or less.

Small molecules of the invention may be made or selected by several methods known in the art and by methods as described herein. Screening procedures can be used to identify small molecules from libraries which bind Yap, or any other protein that interacts with Yap, for example, Shh, CDK7.

### Peptidic Molecules

In another aspect of the invention, the agent that inhibits the expression and/or activity of Yap is a peptidic molecule.

In one embodiment, the peptidic moieties of the invention may comprise an entire protein domain of Yap. In some embodiments, the peptidic moieties of the invention may have as little as 50% identity to Yap, e.g., a peptidic moiety of the invention may be at least 50% identical, at least 60% identical, at least 70% identical, at least 80% identical, at least 90% identical, or at least 95%, 96%, 97%, or 98% identical to Yap.

In another embodiment, the peptidic moieties of the invention may comprise an entire protein domain of any other protein that interacts with Yap, e.g., Shh or CDK7. In some embodiments, the peptidic moieties of the invention may have as little as 50% identity to the protein that interacts with Yap, e.g., Shh or CDK7, for example, a peptidic moiety of the invention may be at least 50% identical, at least 60% identical, at least 70% identical, at least 80% identical, at least 90% identical, or at least 95%, 96%, 97%, or 98% identical to the protein that interacts with Yap, e.g., Shh or CDK7.

A peptidic moiety of the invention may bind to contiguous or non-contiguous amino acid residues of Yap, or any other protein that interacts with Yap, e.g., Shh or CDK7.

A peptide molecule of the invention may be further modified to increase its stability, bioavailability or solubility. For example, one or more L-amino acid residues within the peptidic molecules may be replaced with a D-amino acid residue. The term “mimetic” as applied to the peptidic molecules of the present invention is intended to include molecules that mimic the chemical structure of a D-peptidic structure and retain the functional properties of the D-peptidic structure. The term “mimetic” is further intended to encompass an “analogue” and/or “derivative” of a peptide as described below. Approaches to designing peptide analogs, derivatives and mimetics are known in the art. For example, see Farmer, P. S. in Drug Design (E. J. Ariens, ed.) Academic Press, New York, 1980, vol. 10, pp. 119-143; Ball and Alewood (1990) J. Mol. Recognition 3: 55; Morgan and Gainor (1989) Ann. Rep. Med. Chem. 24: 243; and Freidinger (1989) Trends Pharmacol. Sci. 10: 270. See also Sawyer (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith et al. (1995) J. Am. Chem. Soc. 117: 11113-11123; Smith et al. (1994)J. Am. Chem. Soc. 116: 9947-9962; and Hirschman et al. (1993)J. Am. Chem. Soc. 115: 12550-12568.

Other methods to stabilize peptides and peptide structures may be used, e.g., olefinic cross-linking of helices through O-allyl serine residues (Blackwell, H. E.; Grubbs, R. H. Angew. Chem., Int. Ed. 1998, 37, 3281-3284, incorporated herein by reference), all-hydrocarbon cross-linking (Schafmeister and Verdine J. Am. Chem. Soc. 2000, 122 (24), 5891-5892, incorporated herein by reference) and the methods disclosed in U.S. Pat. No. 7,183,059 (incorporated herein by reference). The methods disclosed in Blackwell et al. and Schafmeister et al. may be described as producing “stapled” peptides, i.e., peptides which are covalently locked into a particular conformational state or secondary structure, or peptides which have a particular intramolecular covalent linkage which predisposes them to form a particular conformation or structure. If a peptide thus treated is predisposed to, e.g., form an alpha-helix which is important for target binding, then the energetic threshold for binding will be lowered. Such “stapled” peptides have been shown to be resistant to proteases and may also be designed to cross the cellular membrane more effectively (also see Walensky et al. Science 2004: Vol. 305. no. 5689, pp. 1466-1470; Bernal et al. J Am Chem Soc. 2007, 129(9):2456-7 which are incorporated herein by reference). Accordingly, peptides of the invention may be thus stapled or otherwise modified to lock them into a specific conformational shape or they may be modified to be predisposed to particular conformation or secondary structure which is beneficial for binding. It is contemplated that such peptide modifications may occur prior to peptide selection such that the benefit of any conformational constraints may also be selected for. Alternatively, in some embodiments, the modifications may be made after selection to preserve a conformation known to be beneficial to binding or to further enhance a peptide candidate. See also, WO/2010/033617, the entire contents of which are incorporated herein by reference.

Other methods to stabilize peptides and peptide structures include linking the amino and carboxy termini of a protein with a peptide bond to form a circular or cyclic peptide. See, e.g., WO/2008/07489 and U.S. Pat. No. 55,726,287, the entire contents of each of which are incorporated herein by reference.

As used herein, a “derivative” of a peptidic molecule of the invention refers to a form of the peptidic molecule in which one or more reaction groups on the molecule have been derivatized with a substituent group. Examples of peptide derivatives include peptides in which an amino acid side chain, the peptide backbone, or the N or C terminus has been derivatized (e.g., peptidic compounds with methylated amide linkages). As used herein an “analogue” of a peptidic molecule of the invention refers to a peptidic molecule that retains chemical structures of the molecule necessary for functional activity of the molecule yet also contains certain chemical structures that differ from the molecule. An example of an analogue of a naturally-occurring peptide is a peptide that includes one or more non-naturally-occurring amino acids. As used herein, a “mimetic” of a peptidic molecule of the invention refers to a peptidic molecule in which chemical structures of the molecule necessary for functional activity of the molecule have been replaced with other chemical structures that mimic the conformation of the molecule. Examples of peptidomimetics include peptidic compounds in which the peptide backbone is substituted with one or more benzodiazepine molecules (see e.g., James et al. (1993) Science 260: 1937-1942).

Analogues of the peptidic molecules of the invention are intended to include molecules in which one or more L- or D-amino acids of the peptidic structure are substituted with a homologous amino acid such that the properties of the molecule are maintained. Preferably conservative amino acid substitutions are made at one or more amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Non-limiting examples of homologous substitutions that can be made in the structures of the peptidic molecules of the invention include substitution of D-phenylalanine with D-tyrosine, D-pyridylalanine or D-homophenylalanine, substitution of D leucine with D-valine or other natural or non-natural amino acid having an aliphatic side chain and/or substitution of D-valine with D-leucine or other natural or non-natural amino acid having an aliphatic side chain.

The term “mimetic,” and in particular, “peptidomimetic,” is intended to include isosteres. The term “isostere” as used herein is intended to include a chemical structure that can be substituted for a second chemical structure because the steric conformation of the first structure fits a binding site specific for the second structure. The term specifically includes peptide back bone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, indicates the absence of an amide bond. The structure that replaces the amide group is specified within the brackets.

Other possible modifications include an N-alkyl (or aryl) substitution (ψ[CONR]), or backbone crosslinking to construct lactams and other cyclic structures. Other derivatives of the modulator compounds of the invention include C-terminal hydroxymethyl derivatives, 0-modified derivatives (e.g., C-terminal hydroxymethyl benzyl ether), N-terminally modified derivatives including substituted amides such as alkylamides and hydrazides.

Peptidic molecules of the present invention may be made by standard methods known in the art. The peptidic molecule may be cloned from human cells using standard techniques, inserted in to a recombinant vector, and expressed in an in vitro cell system (e.g., by transfection of the vector into yeast cells). Alternatively, the peptidic molecules may be designed and synthesized de novo via known synthesis methods such as Atherton et al. (1989) Oxford, England: IRL Press. ISBN 0199630674; Stewart et al. (1984) 2nd edition, Rockford: Pierce Chemical Company, 91. ISBN 0935940030; Merrifield (1963) J. Am. Chem. Soc. 85: 2149-2154.

The peptidic molecules can then be tested for functional activity using any of the assays described herein, e.g., those described in the Examples section below.

Screening Methods

In certain aspect, the presentation provides a method for identifying a compound that inhibits heterotopic ossification (HO) of a cell. The method comprises providing a cellular indicator composition, contacting the indicator composition with a test compound, determining the effect of the compound on the expression and or activity of YAP in the indicator composition, wherein a decrease in the expression and/or activity of Yap indicates that the test compound inhibits HO, thereby identifying a compound that inhibits HO of the cell.

In some embodiments, the indicator composition comprises a mesenchymal progenitor cell, a mesenchymal stem cell, a mesenchyme derived differentiated cell or a heterotopic ossified cell. The cells suitable for the methods of the invention may comprise an inactive Gnas gene, or a constitutively active caALK2 gene. Alternatively, the cell may comprise an ACVR^{R206H} gene.

In addition to have an effect on the expression and/or activity of Yap in the indicator composition, the test compound may also have an effect on the expression and/or activity of several other markers, e.g., Osx, Col1α1, Opn, Runx2, Shh, Ihh, Ctgf, Cyr61, Ankdr1, Ptch1, Gli1, Hip, Taz, Tgfβ 1, Tgfβ2 and Tgfβ3. Accordingly, the methods of the present invention further comprise determining the expression and/or activity of these markers in order to identify a compound that inhibits HO of a cell.

Other well known methods that may be used to identify molecules from libraries which bind Yap, or any other protein that interacts with Yap, for example, Shh, CDK7, include methods that utilize libraries in which the library members are tagged with an identifying label, that is, each label present in the library is associated with a discreet compound structure present in the library, such that identification of the label tells the structure of the tagged molecule. One approach to tagged libraries utilizes oligonucleotide tags, as described, for example, in PCT Publication No. WO 2005/058479 A2 (the Direct Select technology) and in U.S. Pat. Nos. 5,573,905; 5,708,153; 5,723,598, 6,060,596 published PCT applications WO 93/06121; WO 93/20242; WO 94/13623; WO 00/23458; WO 02/074929 and WO 02/103008, and by Brenner and Lerner (1992) Proc. Natl. Acad. Sci. USA 89: 5381-5383; Nielsen and Janda (1994) Methods Enzymol. 6: 361-371; and Nielsen et al. (1993) J. Am. Chem. Soc. 115: 9812-9813, the entire contents of each of which are incorporated herein by reference in their entirety. Such tags can be amplified, using for example, polymerase chain reaction, to produce many copies of the tag and identify the tag by sequencing. The sequence of the tag then identifies the structure of the binding molecule, which can be synthesized in pure form and tested for activity.

Preparation and screening of combinatorial chemical libraries is well known to those skilled in the art. Such combinatorial chemical libraries, which may be used to identify moieties of the invention, include, but are not limited to, peptide libraries (see, e.g., U.S. Pat. No. 5,010,175, Furka (1991) Int. J. Pept. Prot. Res. 37: 487 493 and Houghton et al. (1991) Nature 354: 84 88). Other chemistries for generating chemical diversity libraries are well known in the art and can be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al. (1993) Proc. Nat. Acad. Sci. USA 90: 6909-6913), vinylogous polypeptides (Hagihara et al. (1992) J. Am. Chem. Soc. 114: 6568), nonpeptidal peptidomimetics with glucose scaffolding (Hirschmann et al. (1992)J. Am. Chem. Soc. 114: 9217 9218), analogous organic syntheses of small compound libraries (Chen et al. (1994) J. Am. Chem. Soc. 116: 2661), oligocarbamates (Cho et al. (1993) Science 261: 1303), and/or peptidyl phosphonates (Campbell et al. (1994) 1 Org. Chem. 59: 658), nucleic acid libraries (see Ausubel, Berger and Russell & Sambrook, all supra), peptide nucleic acid libraries (see, e.g., U.S. Pat. No. 5,539,083), carbohydrate libraries (see, e.g., Liang et al. (1996) Science 274: 1520 1522 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like). Each of the foregoing publications is incorporated herein by reference. Public databases are also available and are commonly used for small molecule screening, e.g., PubChem (pubchem.ncbi.nlm.nih.gov), Zinc (Irwin and Shoichet (2005) J. Chem. Inf. Model. 45: 177-182), and ChemBank (Seiler et al. (2008) Nucleic Acids Res. 36: D351-D359).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.). Moreover, since screening methodologies are so well defined, it is common to contract specialist firms to identify particular compounds for a target of interest (e.g., BioFocus DPI (biofocus.com), and Quantum Lead (q-lead.com)).

Other methods of selecting small molecules which are well known in the art, and may be applied to the methods of the present invention are Huang and Schreiber (1997) Proc. Natl. Acad. Sci. USA 94: 13396-13401; Hung et al. (2005) Science 310: 670-674; Zhang et al. (2007) Proc. Natl. Acad. Sci. USA 104: 4606-4611; or any of the methods reviewed in Gordon (2007) ACS Chem. Biol. 2: 9-16, all of which are incorporated herein by reference in their entirety.

In addition to experimental screening methods, small molecules of the invention may be selected using virtual screening methods. Virtual screening technologies predict which small molecules from a library will bind to a protein, or a specific epitope therein, using statistical analysis and protein docking simulations. Most commonly, virtual screening methods compare the three-dimensional structure of a protein to those of small molecules in a library. Different strategies for modeling protein-molecule interactions are used, although it is common to employ algorithms that simulate binding energies between atoms, including hydrogen bonds, electrostatic forces, and van der Waals interactions. Typically, virtual screening methods can scan libraries of more than a million compounds and return a short list of small molecules that are likely to be strong binders. Several reviews of virtual screening methods are available, detailing the techniques that may be used to identify small molecules of the present invention (Engel et al. (2008) J Am. Chem. Soc. 130, 5115-5123; McInnes (2007) Curr. Opin. Chem. Biol. 11: 494-502; Reddy et al. (2007) Curr. Protein Pept. Sci. 8: 329-351; Muegge and Oloff (2006) Drug Discov. Today 3: 405-411; Kitchen et al. (2004) Nat. Rev. Drug Discov. 3, 935-949). Further examples of small molecule screening can be found in U.S. 2005/0124678, which is incorporated herein by reference.

In some embodiments, small molecules identified as “hits” (e.g., small molecules that demonstrate activity in a method described herein) in a first screen are selected and optimized by being systematically altered, e.g., using rational design, to optimize binding affinity, avidity, specificity, or other parameter. Such potentially optimized structures can also be screened using the methods described herein. Thus, in one embodiment, the invention includes screening a first library of agents using a method described herein, identifying one or more hits in that library, subjecting those hits to systematic structural alteration to create one or more second generation compounds structurally related to the hit, and screening the second generation compound. Additional rounds of optimization can be used to identify an agent with a desirable therapeutic profile.

Agents identified as hits can be considered candidate therapeutic compounds, useful in treating heterotopic ossification disorders described herein. Thus, the invention also includes compounds identified as “hits” by a method described herein, and methods for their administration and use in the treatment, prevention, or delay of development or progression of a disease described herein.

In one embodiment, the inhibitor of Yap acts directly on Yap and inhibits its function. In another embodiment, the inhibitor of Yap acts indirectly, e.g., through another molecule which interacts with Yap, e.g., Shh or CDK7, thereby decreasing Yap expression and/or activity. In yet another embodiment, the inhibitor of Yap acts through the Hedgehog pathway, e.g., by acting on a component of the Hedgehog pathway, for example, Shh, Desert Hedgehog (Dhh), Indian Hedgehog (Ihh), Patched (Ptch1, Ptch2), Gli1, Gli2, Gli3, and Smoothened (Smo).

Exemplary inhibitors of Yap include, but are not limited to, verteporfin (VP), CA3(CIL56), and YAP/TAZ inhibitor-1 as described in WO2017058716A1, the entire content of which is incorporated herein by reference. In some embodiments, the small molecule inhibitor of Yap is verteporfin (VP).

Examples of CDK7 inhibitors are, for example, THZ1, CT7001, BS-181 HCl, PHA-793887, SNS-032 (BMS-387032), Flavopiridol (Alvocidib) and Flavopiridol HCl. In some embodiments, the CDK7 inhibitor comprises THZ1. In other embodiments, the CDK7 inhibitor comprises CT7001.

### IV. Pharmaceutical Compositions

Agents that inhibit the expression and/or the activity of Yes-associated protein (Yap), e.g., inhibitory nucleic acids, small molecule inhibitors, peptic molecules, and/or anti-Yap antibodies, or antigen binding fragments thereof, as described herein, may be formulated into pharmaceutical compositions suitable for administration in human or non-human subjects. Such pharmaceutical compositions may be intended for therapeutic use, or prophylactic use. One or more of the inhibitors, e.g., small molecule inhibitors or antibodies, can be mixed with a pharmaceutically acceptable carrier (excipient), including buffer, to form a pharmaceutical composition for administering to a patient who may benefit from reduced Yap activity in vivo.

“Pharmaceutically acceptable” means that the carrier must be compatible with the active ingredient of the composition (and preferably, capable of stabilizing the active ingredient) and not deleterious to the subject to be treated. Examples of pharmaceutically acceptable excipients (carriers), including buffers, would be apparent to the skilled artisan and have been described previously. See, e.g., Remington: The Science and Practice of Pharmacy 20th Ed. (2000) Lippincott Williams and Wilkins, Ed. K. E. Hoover. Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

In one example, a pharmaceutical composition described herein contains more than one Yap inhibitor, e.g., more than one small molecule inhibitors of Yap, and/or more than one anti-Yap antibody, or antigen-binding portion thereof, that recognize different epitopes/residues of the target antigen.

In some examples, the pharmaceutical composition described herein comprises emulsion-based or lipid-based formulations, such as liposomes containing a Yap inhibitor, e.g., a small molecule inhibitor of Yap, and/or anti-Yap antibody or antigen-binding portion thereof, which can be prepared by any suitable method, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The Yap inhibitor, e.g., a small molecule inhibitor of Yap, and/or an anti-Yap antibody or antigen-binding portion thereof, may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Exemplary techniques have been described previously, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, or antigen-binding portion thereof, which matrices are in the form of shaped articles, e.g. films, or microcapsules. Examples of sustained-release matrices include polyesters, hydrogels (for example, poly(2-hydroxyethyl-methacrylate), or poly(v nylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 mg to about 500 mg of the active ingredient of the present disclosure. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing a Yap inhibitor, e.g., a small molecule inhibitor of Yap, and/or anti-Yap antibody or antigen-binding portion thereof, with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

### V. Kits

The invention also provides compositions and kits for inhibiting, treating or monitoring a heterotopic ossification disease or disorder, recurrence of a heterotopic ossification disorder, or survival of a subject being treated for a heterotopic ossification disorder. These kits may include one or more agents that inhibit the expression and/or activity of Yap (e.g., nucleic acids, small molecules, antibodies, antigen-binding portions thereof, or peptidic molecules) and instructions for use. The kit can further contain one more additional reagent, such as an immunosuppressive reagent, a cytotoxic agent or a radiotoxic agent or one or more additional agents of the invention, as described herein. Kits typically include a label indicating the intended use of the contents of the kit. The term label includes any writing, or recorded material supplied on or with the kit, or which otherwise accompanies the kit.

In certain embodiments, the kits can also comprise, e.g., a buffering agents, a preservative, a protein stabilizing agent, reaction buffers. Each component of the kit can be enclosed within an individual container and all of the various containers can be within a single package, along with instructions for interpreting the results of the assays performed using the kit.

The kits of the invention may optionally comprise additional components useful for performing the methods of the invention.

The present invention is further illustrated by the following examples, which are not intended to be limiting in any way. The entire contents of all references, patents and published patent applications cited throughout this application, as well as the Figures, are hereby incorporated herein by reference.

### EXAMPLES

### Materials and Methods

### Mice

All mouse experiments were approved by the Harvard IACUC. All mice have been previously described in the literature: Gnas^{f/f}, tdTMT^{tg/+}; Yap^{f/f}, Yap^{tg/+}, rtTA^{tg/+}, Shh^{f/f}, caALK2^{f/+}, Scx-Cre⁺, Ptch1-Lacz, CTJF-GFP⁺, Col1a12.3-GFP⁺, ACVR1^{Q207D/+} and ACVR1^{R206H/+} (ACVR1^{FlExR206H/+)} All animal experiments were performed under a protocol approved by the NHGRI Animal Care and Use Committee.

For ATP mouse model, 4-week-old male mice were anesthetized. A 27-gauge needle was punctured into the Achilles tendon body from the lateral aspect percutaneously and this process was repeated six times at different parts of Achilles tendon body for each mouse. For sham operation, the needle was punctured through the skin without touching the Achilles tendons.

Constitutively active ALK2 (caALK2) mice were provided by Dr Yuji Mishina at University of Michigan. To induce expression of caALK2, adenoviral-Cre and cobra venom factor (EMD/Millipore, 233552; 0.03 μg per mouse) were injected into the limbs at 4 weeks of age. Six weeks later, limbs were collected and analyzed.

The ACVR1^{FlExR206H} mice conditionally express ACVR1^{R206H/+} knock-in alleles, were kindly provided by Dr. Aris Economides (Regeneron Pharmaceuticals, Inc.), mated with ScxCre mice, to yield ScxCre; ACVR1^{FlEx206H/+} mice, which represent a FOP mouse model that develops spontaneous HO of tendons, ligaments, and joints. These mice were treated with Verteporfin (VP) (4 mg/kg/d intraperitoneal (IP) injection, 5 days per week) or an equal volume of PBS for 8 weeks, starting at 6 weeks of age. In the POH model, VP (200 μg/ml) or DMSO (vehicle control) were applied topically 5 days per week for 6 weeks to the surface of the legs (hair has been shaved) of the Gnas mutant groups immediately after Ad-Cre injection. In another set of experiments, IP injection of the Gnas mutant mice 5 days a week for 6 weeks after A-Cre injection with VP (2.5 mg/ml) and THZ1 (10 mg/kg). CT7001 (10 mg/kg) was garaged every other day immediately after Ad-Cre injection. Equal volume of PBS was IP injected or garaged as control.

### MicroCT (μCT) Analysis

Mice were fixed overnight in 4% PFA and transferred to 70% ethanol, then analyzed by high-resolution μCT using a deskop μCT (Scanco Medical) according to recommended guidelines. The scanner was set at a voltage of 55 kV and a resolution of 12.3 μm per pixel. The images were reconstructed, analyzed for HO bone volume and endogenous bone mass. Briefly cortical bone parameters and cancellous bone microarchitecture from tibia were determined using a 7-μm isotropic voxel size. For ectopic bone, bone volume was measured. For cancellous bone, bone volume fraction (bone volume per tissue volume [BV/TV]) (percentage), trabecular number (Tb.N) (per millimeter), trabecular thickness (Tb.Th) (millimeter), trabecular separation (Tb.Sp) (millimeter) was analyzed. For cortical bone, cortical area fraction (Ct. BV/TV) (percentage), cortical bone density (Ct. BMD) (mgHA/cm3) were evaluated.

### Alkaline Phosphatase Staining and Quantification.

For ALP staining, cells were fixed in 4% PFA for 20 minutes, washed with PBS for three times, then stained with 1-Step NBT/BCIP (Thermo, 34042) for 30 minutes and washed by PBS. There were three biological replicates for each group. ALP staining was quantified as previously described. Cells were scraped into a radioimmunoprecipitation assay (RIPA) buffer (containing 50 mM Tris-HCl pH 7.5, 150 mM NaCl, 5% Glycerol, 1 mM EDTA, 1% NP-40, 0.1% SDS and 0.25% Na-deoxycholate) and centrifuged. The enzymatic alkaline phosphatase activity in the supernatant of cell lysate was assayed by measuring the chromogenic p-nitrophenol formed from the enzymatic hydrolysis of p-nitrophenyl phosphate (the substrate) by light absorbance at 405 nm. The alkaline phosphatase activity was normalized to total protein, as measured by BCA protein assay. All experiments were performed in triplicates and means and standard deviations were calculated.

### Von Kossa Staining

For Von Kossa staining of cell cultures, cells were fixed in 4% PFA in PBS for 20 minutes, washed in distilled water, and then stained with 5% silver nitrate under a 60 watt lamp for 1 hr. The stained cells were washed in distilled water 3 times, then in 5% sodium thiosulfate for 5 min, and then rinsed in water.

### SMP Isolation and Culture in Osteogenic Media

Subcutaneous mesenchymal progenitor (SMP) cells are potential osteoprogenitor cells within the subcutaneous connective tissue which can be induced to become osteoblast cells and adipocytes. Subcutaneous skin tissue containing adipose deposits was removed under sterile conditions and washed in PBS supplemented with 100 U/mL penicillin and 100 μg/mL streptomycin on ice. The tissue was then minced, and digested with 1 mg/ml collagenase type I and 0.5% Trypsin in 0.1% BSA for 70 minutes at 37° C. The digested tissue was centrifuged at 500 g for 10 min and the pellet was carefully collected after aspirating off the floating fat depots. After a second centrifugation at 500 g for 10 min, the cellular pellet was filtered through a 100 μm mesh filter to remove debris.

### Adenovirus Treatment

Cre recombinase adenovirus from Berk (˜10¹² pfu/ml) were diluted 1:50 in 100 μl PBS solution and injected into the subcutaneous region of the limbs of 4 weeks old mice. Six weeks, 3 months or 8 months after injection, the limbs were harvested from euthanized mice and fixed in 4% PFA overnight. Ectopic bone formation was analyzed by μCT and skeletal preparation. The Cre recombinase or GFP adenovirus (˜10¹⁰ pfu/ml) was diluted 1:2000 to infect cells in vitro. Four hours later, fresh medium was added and 24 hours later, the medium was changed.

### Conditional Medium Experiment

The Gnas^{f/f}, Gnas^{f/f}; Shh^{f/f}, Gnas^{f/f}; Yap^{f/f} SMPs were infected with Ad-GFP or Ad-Cre and cultured with osteogenic medium. The medium was changed every two days and collected after filtration through 0.22 uM filter. Then the conditioned medium was added to wildtype SMP cells cultured for 7 days for ALP assay or 21 days for von Kossa staining.

### Calcium and Phosphorus Measurement

The serum was collected and centrifuged before measurement. Then calcium and phosphorus were measured using a kit (Stanbio Cat #0155-225; Stanbio Cat #0830-125) according to the manufacturer's procedures.

### Q-PCR

The skin was removed from the hindlimb covering the tibia and the subcutaneous tissue area (5 mm (1)×5 mm (w)×4 mm (h)) where the Ad-Cre were locally injected was isolated. Subcutaneous connective tissue and some muscle cells surrounding the ectopic bone would have been included. Total RNA was isolated first with Trizol (Invitrogen). 1st strand cDNA was generated from total RNA (1-3 mg) using SuperScript II Reverse Transcriptase with random primer (Life Technologies). qPCR was performed using an AB17900 light cycler and 40 cycles of 95° C. 15 seconds and 60° C. for 60 seconds. PCR product accumulation was detected using Sybr green (Invitrogen; Platinum SYBR Green qPCR SuperMix-UDG). Expression levels were always given relative to glyceraldehyde 3-phosphate dehydrogenase (Gapdh). Primers used for amplification are Ptch1:

Forward
5′-CTC TGG AGC AGA TTT CCA AGG-3′.
Reverse
5′-TGC CGC AGT TCT TTT GAA TG-3′;
Gli1: Forward
5′-GAA AGT CCT ATT CAC GCC TTG A-3′,
Reverse
5′-CAA CCT TCT TGC TCA CAC ATG TAA G-3′;
Hhip: Forward
5′-GGG AAA AAC AGG TCA TCA GC-3′,
Reverse
5′-ATC CAC CAA CCA AAG GGC-3′.
Osx: Forward
5′-CCC ACT GGC TCC TCG GTT CTC TCC-3′,
Reverse
5′-GCTBGAA AGG TCA GCG TAT GGC TTC-3′;
Col1a1: Forward
5′-CAC CCT CAA GAG CCT GAG TC-3′,
Reverse
5′-GTT CGG GCT GAT GTA CCA GT-3′.

The results were calculated based on three biological replicates.

### Immunohistochemistry

Tissues were fixed in 4% paraformaldehyde in phosphate-buffered saline (PBS) and processed for cryostat section according to standard protocol. Frozen sections were placed at room temperature for 15 minutes. Slides were placed in 0.5% Triton-X100 for 15 minutes, equilibrated in phosphate buffered saline with 0.1% Tween-20 (PBS-T), and blocked for 1 hour with 5% normal goal serum in PBS-T. Different primary antibody were applied overnight at 4° C., sections were incubated with universal secondary antibody for 60 min, room temperature. The sections were mounted in mounting medium with DAPI (Sigma, F6057).

### X-Gal Staining

The hind limbs were dissected with the skin removed, and then fixed in 0.5% formaldehyde and 0.1% glutaraldehyde for 1 h. X-gal staining was performed according to previously described procedures (Guo, X., et al. Genes Dev 18, 2404-2417 (2004)).

### Immunoblotting

Tissue or cell lysates were prepared using a lysis buffer [20 mM Tris (pH 7.4), 150 mM NaCl, 1% Tri-ton X-100, 1 mM EDTA, 1 mM EGTA, 2.5 mM sodium pyrophosphate, 1 mM β-glycerophosphate, 1 mM sodium orthovanadate] or RIPA buffer (Santa Cruz Biotechnology), respectively, containing protease inhibitor mixture (Roche). Total cell lysates were analyzed by Western blotting according to standard procedures.

### RNA Sequencing

The Gnas^{f/f} SMPs were infected with Ad-GFP or Ad-Cre and cultured for 7 days with osteogenic medium before total RNA isolation. Two replications were prepared for each group. The libraries were constructed using Ion AmpliSeq Transcriptome Mouse Gene Expression Panel, Chef-Ready Kit according to the manufacturer's protocols. The library qualities were checked using a BioAnalyzer 2100 and the concentrations were determined from the analysis profiles. Six barcoded libraries were pooled together on an equimolar basis and sequenced using the Ion 550 Chip Kit. Genes with a change fold ≥2.82 or ≤−0.35 and p<0.05 by 1-way ANOVA with F test were identified as differentially expressed genes and analyzed using the DAVID Bioinformatics Resources 6.8.

### ATAC Sequencing

The Gnas^{f/f} SMPs were infected with Ad-GFP or Ad-Cre and cultured for 7 days with osteogenic medium. Two replicates were prepared for each group. ATAC-seq were performed by following Omni-ATAC protocol as described before. Briefly, Nuclei were isolated from 50,000 cells for transposition reaction with Tn5 Transposase (Illumina, catalog #FC-121-1030). DNA were purified by using PCR purification kit (Qiagen). To generate sequencing libraries, purified DNA was amplified with NEBNext high-fidelity 2×PCR Master Mix (New England Biolabs). Sequencing libraries was sequenced using Illumina Nextseq 550 sequencer to obtain 150 bp paired-end reads at the Biopolymer Facility of Harvard Medical School.

### ATAC-Seq Analysis

All sequencing reads from ATAC-seq were trimmed by Cutadapt to remove adapter sequence. Trimmed reads were aligned to the mouse genome (Mm10, Genome Reference Consortium GRCm38) using Bowtie2. Peak-calling were performed using Genrich (https://github.com/jsh58/Genrich) with ATAC-seq mode, PCR duplicates and mitochondrial reads were excluded during peak-calling. Identified peaks from replicate data were subsequently combined and processed. ATAC-seq signal tracks were generated using ‘bamcoverage’ function in Deep tools and presented by Integrative Genomics Viewer (IGV) software. Peak comparison, annotation of motif enrichment was performed using HOMER v4.10. Ontologies of genes were identified by DAVID.

### Chromatin Immunoprecipitation Quantitative PCR (ChIP-qPCR)

The Gnas^{f/f} SMPs were infected with Ad-GFP or Ad-Cre and cultured for 7 days in osteogenic medium. ChIP assays were performed using the ChIP Kit (Active Motif, 53001/53004). Chromatin was immunoprecipitated using anti-Yap (CST, Cat #140745) antibody. Anti-Histone H3 antibody (CST) and normal rabbit IgG (CST) were used as positive control and negative control, respectively. ChIP-derived DNA was quantified using qRT-PCR. CRISPRi/sgRNA plasmid construction. The pLV hU6-sgRNA hUbC-dCas9-KRAB-T2a-Puro plasmid are available from Addgene (71236). Targeted sgRNAs (to the Shh promoter/enhancer) were inserted to the plasmid using BsmBI sites.

### Lentiviral Transduction

HEK293T cells were plated at a density of 5.1×10³ cells/cm² in DMEM supplemented with 10% FBS and 1% penicillin-streptomycin. The next day after seeding, cells were cotransfected with pLV hU6-sgRNA hUbC-dCas9-KRAB-T2a-Puro plasmid (20 μg), the second-generation packaging plasmid psPAX2 (Addgene, 12260, 15 μg), and the envelop plasmid pM@2.G (Addgene, 12259, 6 ug) using Lipofectamine 2000 (Life Technologies). After 14-20 h, the transfection medium was exchanged and conditioned medium containing lentivirus was collected 24 and 48 h after the first media exchange. Residual producer cells were cleared from lentiviral supernatant by filtration through 0.45 um filters. The Gnas mutant and control SMPs were seeded in 24-well plates. The following day each sample was infected with 100 μl of sgRNA-containing lentiviruses. 1 μg/ml puromycin was used to select transduced cells. The cells were cultured in osteogenic medium for 4 days and collected for RNA extraction and qPCR analysis.

### Human Subjects

The study was approved by ethics committee of First Affiliated Hospital of Sun Yat-sen University. Written informed consent was obtained from all subjects. HO identified radiographically were collected from 28 patients (19 male and 9 female, previously healthy, nonsmoking individuals; age ranging from 38 to 79 years), who were diagnosed with ossification in the posterior longitudinal ligament. 5-mm away from the edge of the ossified area was defined as the para-ossified area, and then was used for RNA extraction.

### Sample Size

In all experiments, representative images of several independent samples (N>3) were shown. To show whether there is a significant difference between two sample groups (wild type and transgenic mice with Ad-Cre injection or ATP surgery), we used a two-sample t-test. We have performed Power Analysis to estimate the number of mice needed for our studies based on our previous experience and preliminary data. For instance, the standard deviation (SD) within one genotype or treatment group was approximately 20%. Our goal is to detect a 50% difference between the control group and experimental group in our genetic studies. With a type error of 0.05 (Significance level), type II error of 0.2 (or equivalently power of 80%) and a two sided t test, the sample size would be 3/group (https://www.stat.ubc.ca/˜rollin/stats/ssize/n2.html). If the SD is >20%, we would need >3 mice/group.

### Statistical Analysis

Quantifications were done from at least three independent experimental groups. Statistical analysis between groups was performed by two-tailed Student's t test to determine significance when only two groups were compared. One-way ANOVA with Tukey's post-hoc tests were used to compare differences between multiple groups. p-Values of less than 0.05 and 0.01 were considered significant. Error bars on all graphs are presented as the SD of the mean unless otherwise indicated.

### Example 1. Progressive Expansion of Ectopic Bone in POH is Contributed by Recruitment of Wild Type Cells

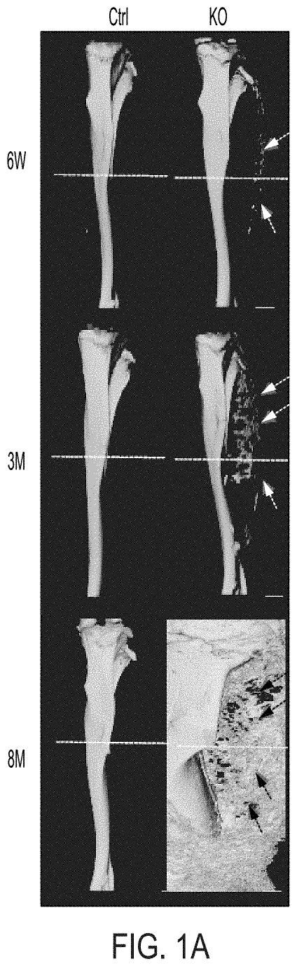

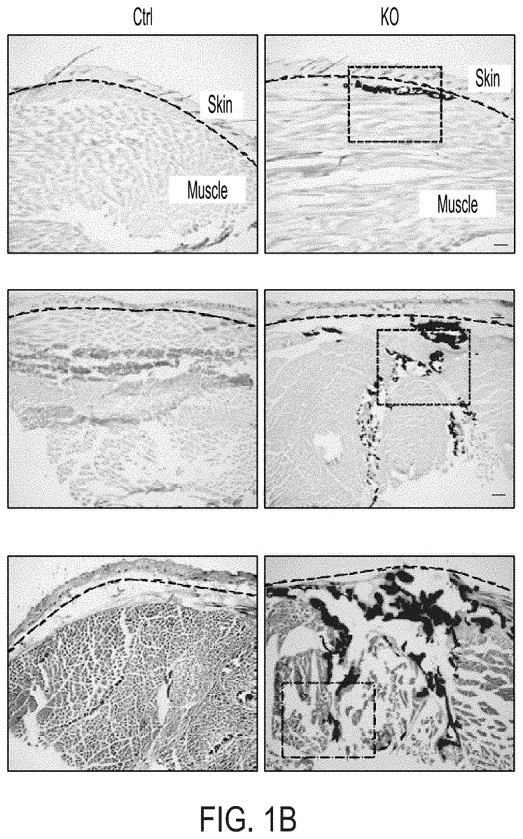

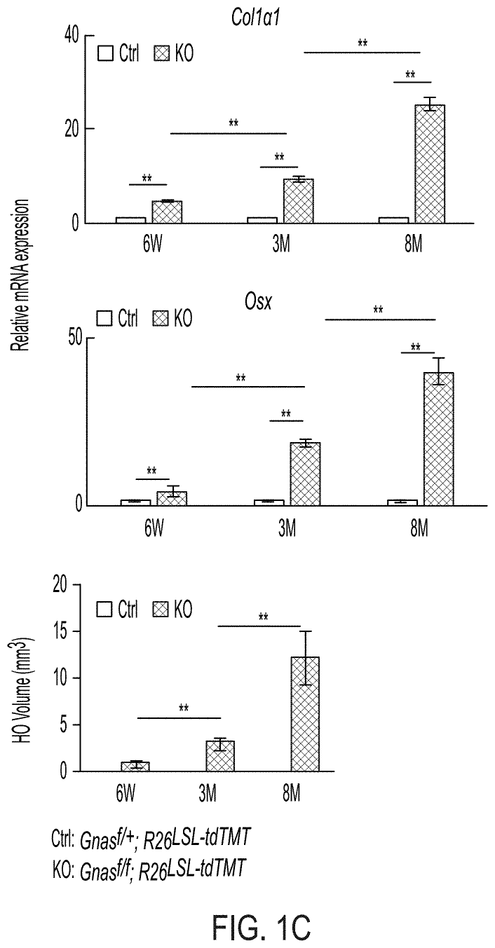

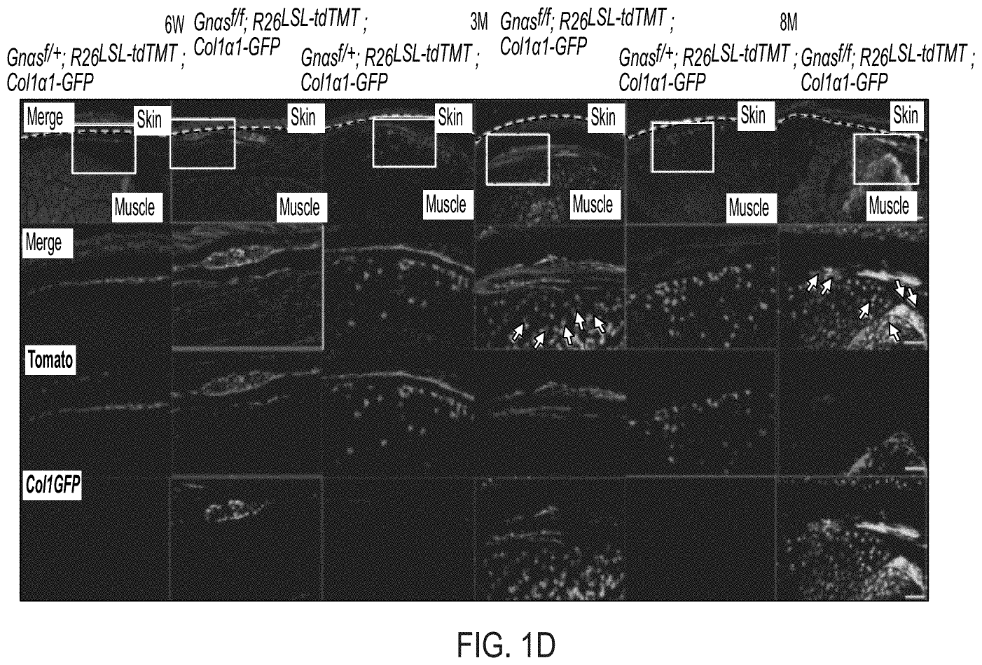

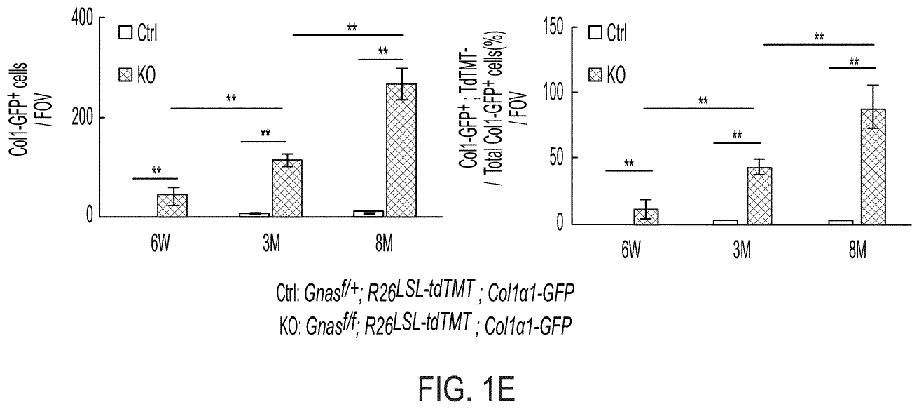

As most heterotopic ossification occurs in adults, a POH mouse model was established by subcutaneously injecting Ad-Cre to the hind limbs of 4-weeks-old Gnas^{f/f} mice, which allows Cre-mediated Gαₛ inactivation by deleting exon 1 of the Gnas gene (Regard, J. B., et al. Nat Med 19, 1505-1512 (2013)). A critical feature of POH is progressively increased ectopic bone formation, which causes increased pain and motion difficulties. In the Ad-Cre injected Gnas^{f/f} mice, ectopic bone formation was examined by μCT scanning and von Kossa staining of the tissue sections (FIGS. 1A and 1B). From 6 weeks to 3 and 8 months post injection, HO expanded progressively from mostly subcutaneous regions, where the Gnas gene deletion was induced by Ad-Cre, to deeper muscle areas (FIGS. 1A and 1B). Quantitative-RT-PCR (QRT-PCR) analysis of osteoblast markers Osterix (Osx) and Collagen 1a1 (Col1a1) also showed that expression of both progressively increased from 6 weeks to 8 months (FIG. 1C).

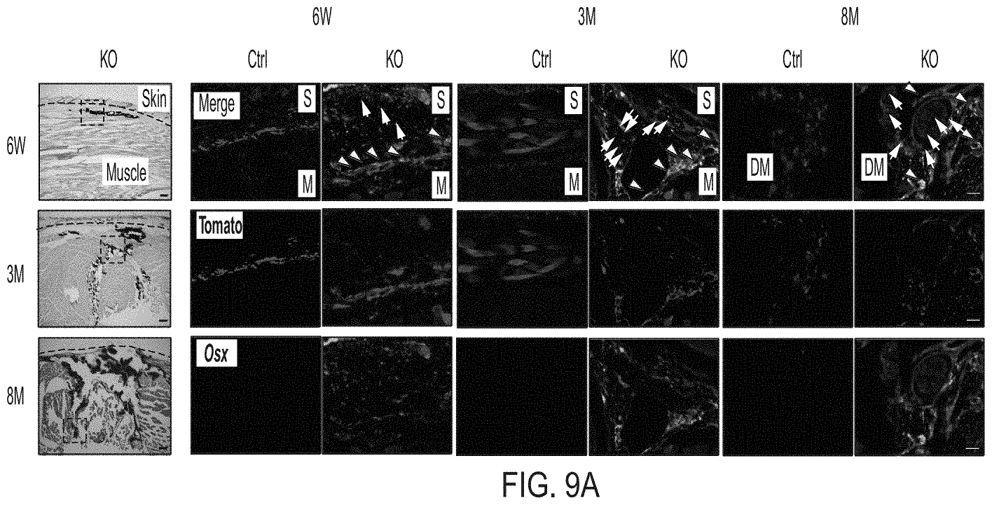

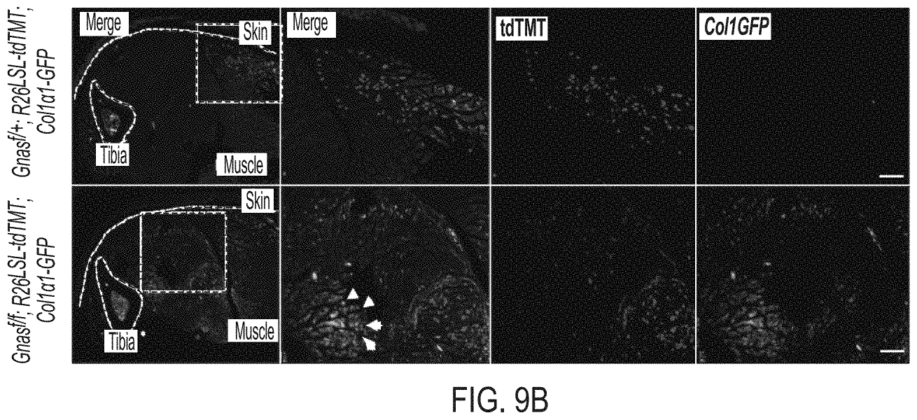

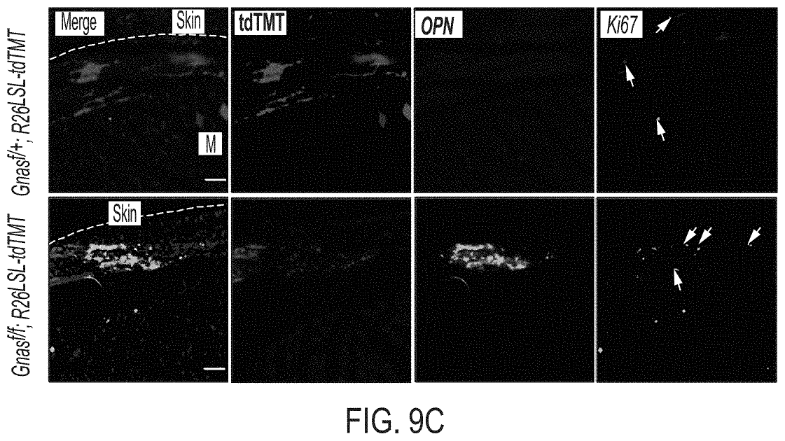

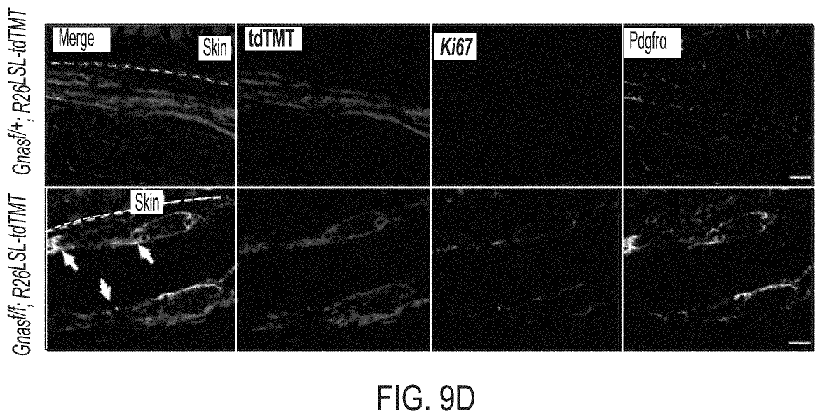

To understand the underlying cellular mechanisms, the cause of HO expansion was investigated, specifically, whether HO expansion is caused by expansion of Gnas^{-/-} cells or progressive recruitment of wild type (WT) cells to the forming bone by the Gnas^{-/-} cells. To distinguish these two possibilities, the Gnas^{-/-} cells were labelled with tdTomato (tdTMT) expression using the Ai9 (R26^{LSL-tdTMT)} mouse strain by generating the Gnas^{f/f}; R26^{LSL-tdTMT} mice (Madisen, L., et al. Nat Neurosci 13, 133-140 (2010)). Injecting Ad-Cre to the Gnas^{f/f}; R26L^{SL-tdTMT} mice allows simultaneous deletion of Gnas and expression of the tdTMT marker. Ectopic osteoblast differentiation was examined at 6 weeks, 3 and 8 months after subcutaneous injection by immunofluorescent Osx staining (FIG. 9A). While many of the Osx⁺ cells were also tdTMT and localized in the subcutaneous region 6 weeks post injection, many of the Osx⁺ cells were tdTMT⁻ cells 3 and 8 months post injection in deeper muscle areas (FIG. 9A). The fraction of these Osx⁺; tdTMT⁻ cells appeared to further increase 8 months post injection, suggesting that ectopic osteoblast differentiation could be controlled by Gnas^{-/-} cells non-cell autonomously.

To further determine the non-cell autonomous function of Gnas^{-/-} cells, lineage tracing experiments were performed and osteoblast cells were genetically labelled using the Col1a12.3-GFP line (Kalajzic, I., et al. J Bone Miner Res 17, 15-25 (2002)) (FIG. 1D). GFP⁺ osteoblast cells were detected in the endogenous bone (tibia) (FIG. 9B). In the soft tissue, GFP⁺ osteoblast cells were only detected after Ad-Cre injection and their numbers were increased between 6 weeks to 3 and 8 months post injection (FIGS. 1D and 1E). Interestingly, many of GFP⁺ cells were not tdTMT⁺ (FIG. 1D). While tdTMT⁺ cells were similar between Ad-Cre injected Gnas^{f/+} and Gnas^{f/f} mice, the number of GFP⁺ cells were progressively increased from 6 weeks to 3 and 8 months (FIG. 1E). Interestingly, many GFP+ cells were not tdTMT+, particularly in the Gnas^{f/f} mice 3 or 8 months post injection (FIG. 1D). Therefore, progressively more tdTMT⁻ WT cells were induced by Gnas^{-/-} cells to become osteoblast cells, contributing to progressive HO expansion.

In addition, Gnas loss also increased cell proliferation shown by Ki67 staining. At an early stage of HO induction (6 weeks after Ad-Cre injection), some of the Ki67+ cells expressed an osteoblast marker Osteopontin (Opn). Most of the Ki67+ cells were also tdTMT+ and some of the Ki67+ cells were also tdTMT⁻ (FIG. 9C). Therefore, induced by Gnas^{-/-} cells, progressively more tdTMT⁻ WT cells were differentiated into osteoblast cells, contributing to the progressive HO expansion in POH. As platelet-derived growth factor receptor α (Pdgfrα) marks fibro/adipogenic progenitor (FAP) cells which contribute to ectopic bone formation, we examined whether the Ki67+ cells were Pdgfra+ cells and indeed most Ki67+ cells were Pdgfra+ and loss of Gnas led to increased Pdgfra+ cell numbers (FIG. 9D).

### Example 2. Gnas Loss Induces Bone Formation Non-Cell Autonomously by Inducing Shh Expression

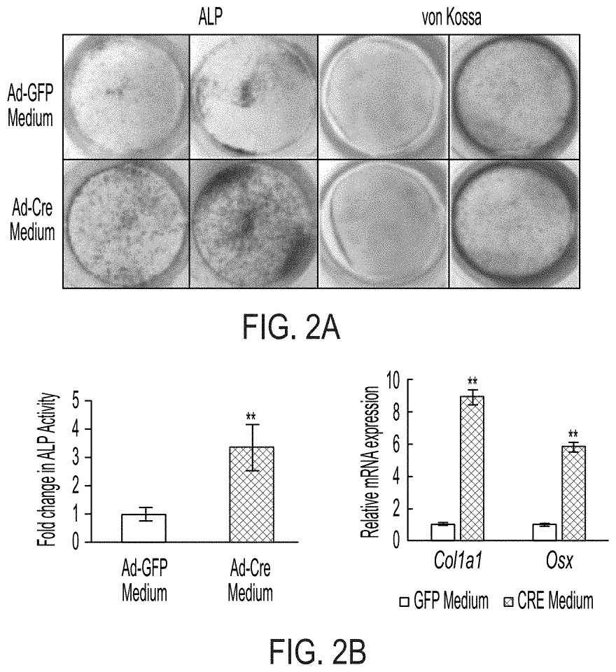

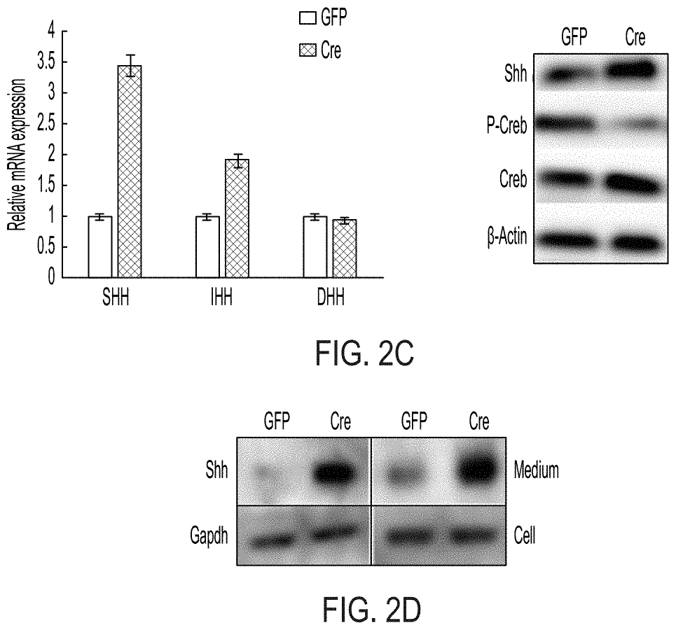

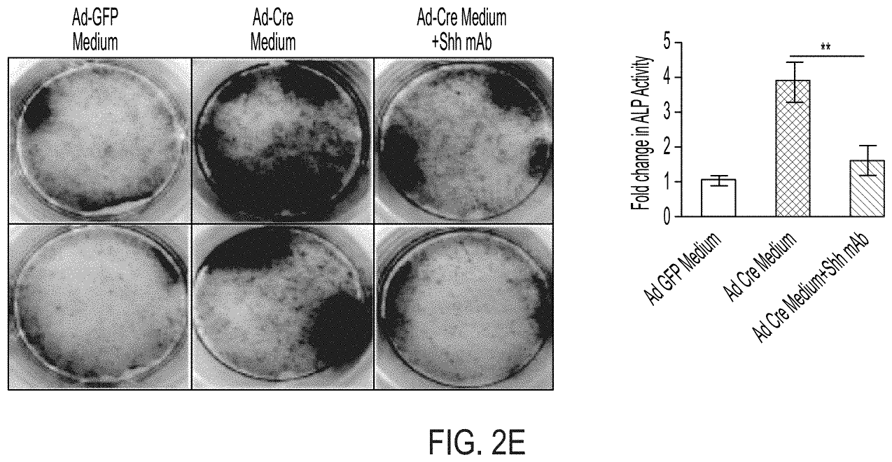

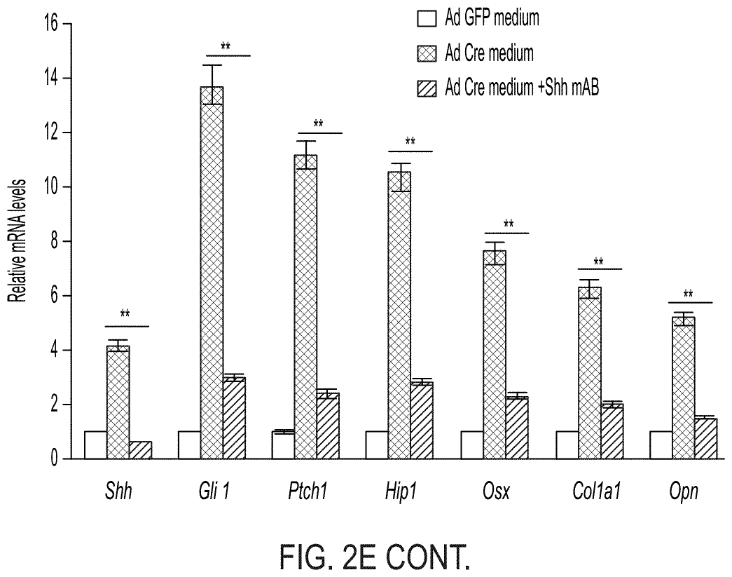

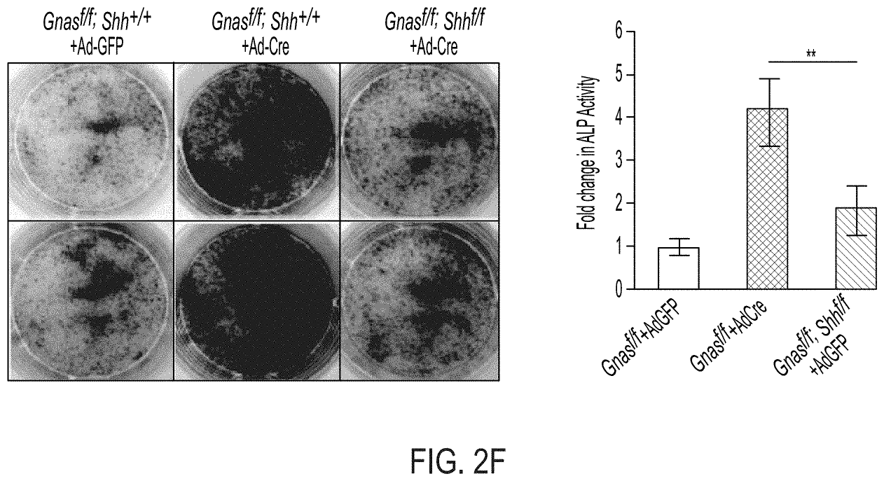

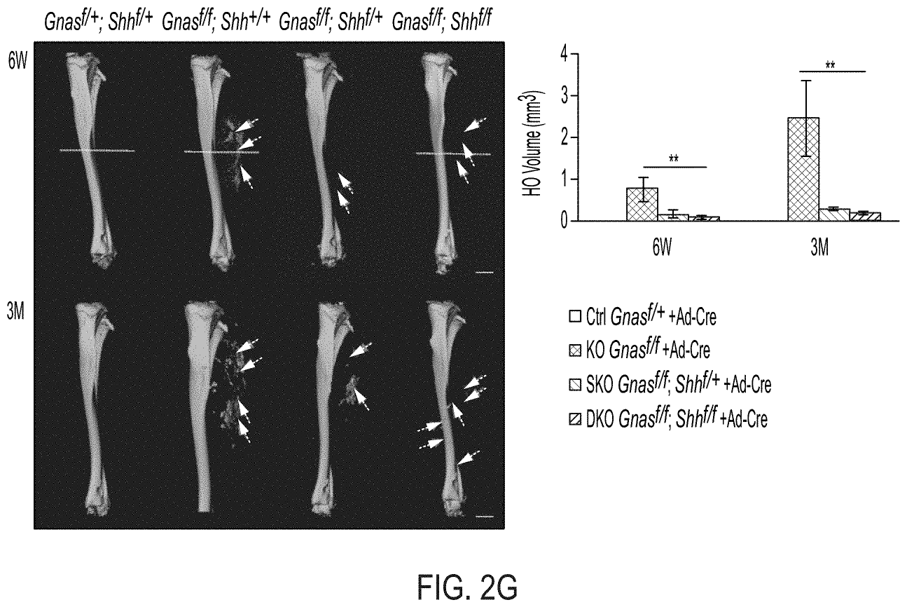

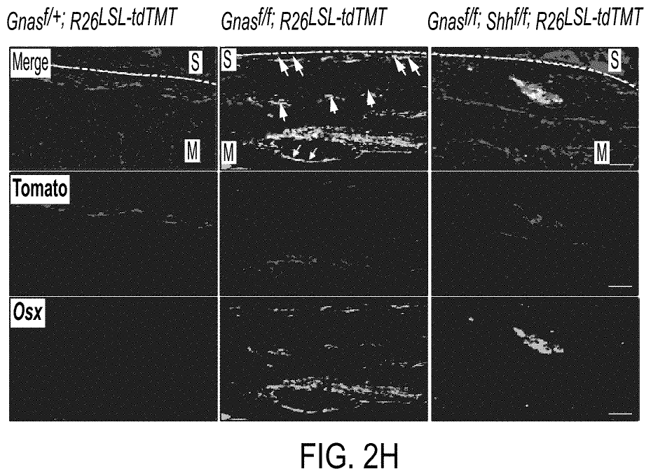

To identify the molecular mechanism underlying the cell non-autonomous function of Gnas^{-/-} cells in osteoblast differentiation, WT SNIP cells were cultured with conditioned medium (CM) collected from Ad-GFP or Ad-Cre infected Gnas^{f/f} SNIP cells. Alkaline phosphatase (ALP) and Von Kossa staining was performed to determine osteogenic differentiation and matrix mineralization 7 and 21 days later, respectively (FIG. 2A), and both were enhanced by CM from the Gnas^{-/-} cells. In addition, expression of osteoblast differentiation markers such as Osx and Col1α1 were upregulated (FIG. 2B). These results showed that the Gnas^{-/-} osteoblasts secreted factors to induce osteoblast differentiation of WT cells.

Previous studies showed that activated Hh signaling was both necessary and sufficient to induce HO in POH (Regard, J. B., et al. Nat Med 19, 1505-1512 (2013)). Hh signaling is controlled non-cell autonomously by Hh ligands (Ingham, P. W. & McMahon, A. P. Genes Dev 15, 3059-3087 (2001)), which are Sonic hedgehog (Shh), Indian hedgehog (Ihh) and Desert hedgehog (Dhh) in mammals. All Hh ligands signal through the same pathway, but determined by their expression patterns in the developing limb bud, Shh acts at early stages to regulate patterning and growth (Echelard, Y., et al. Cell 75, 1417-1430 (1993); Riddle, R. D., Johnson, R. L., Laufer, E. & Tabin, C. Cell 75, 1401-1416 (1993); Zhu, J., et al. Dev Cell 14, 624-632 (2008)), while Ihh acts later during endochondral bone formation to not only control chondrocyte proliferation and hypertrophy, but also induce osteoblast differentiation from mesenchymal progenitor cells in the perichondrium (Kronenberg, H. M. Nature 423, 332-336 (2003)).

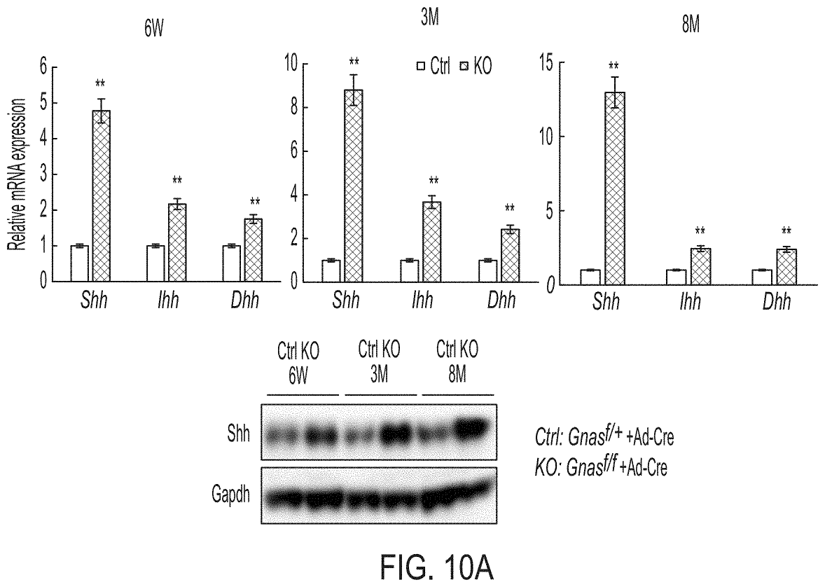

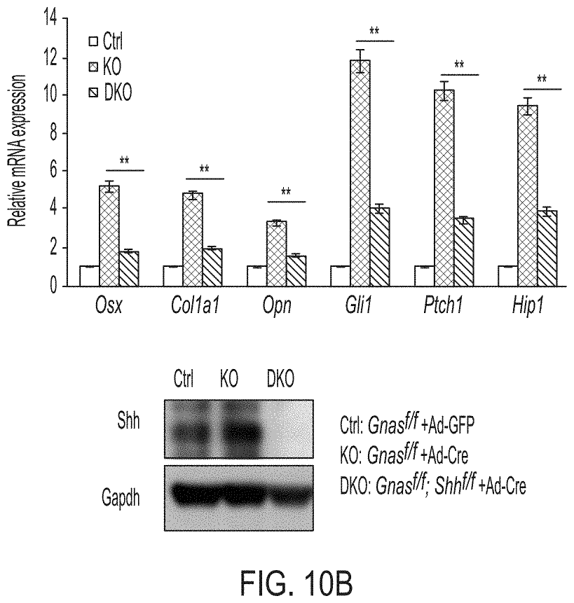

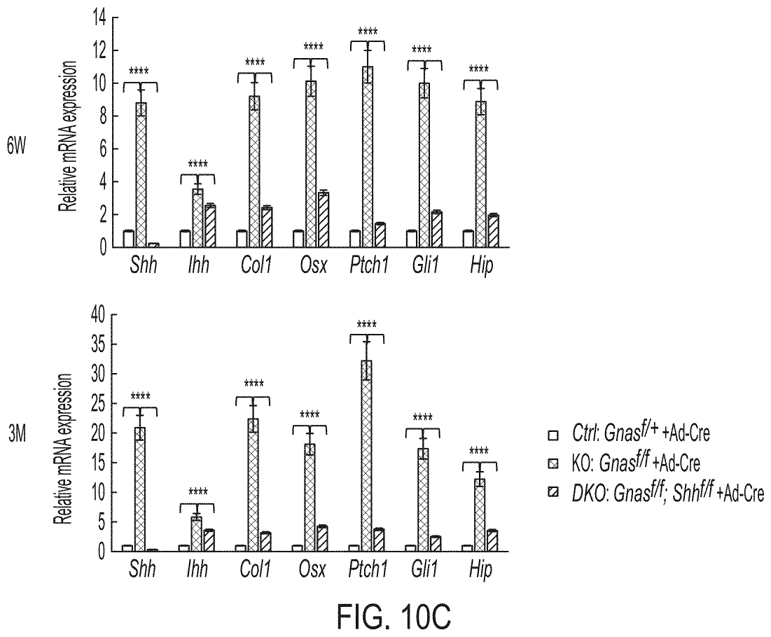

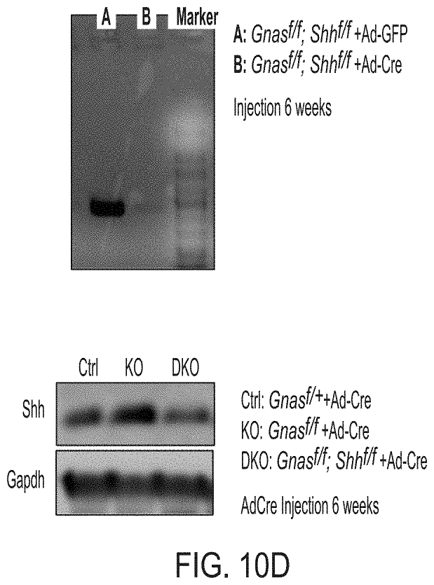

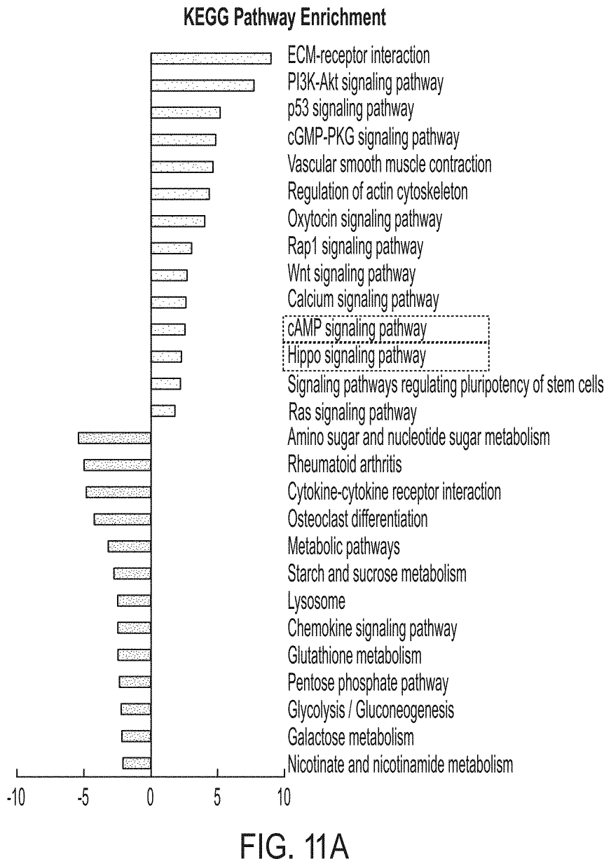

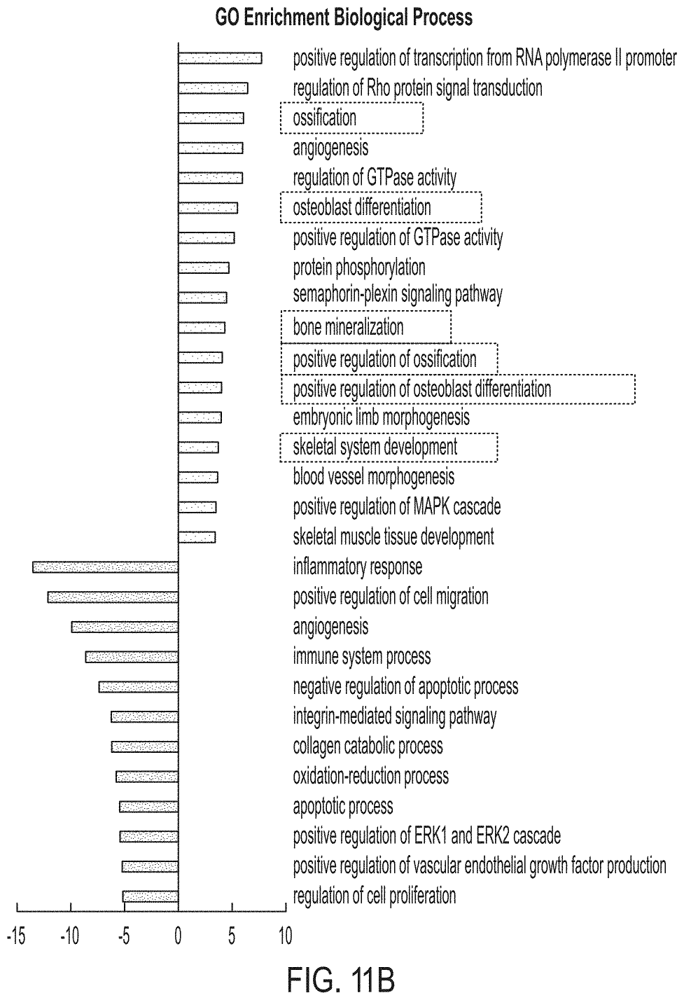

To test whether the WT cells were induced to become osteoblast cells by Hh signaling activation, expression of Shh, Ihh and Dhh were first examined by QRT-PCR. Shh expression was found to be most upregulated and Dhh expression did not change (FIG. 2C). Furthermore, Western blotting analyses showed that Shh protein was much increased in the Gnas^{-/-} CM (FIG. 2D) and a neutralizing monoclonal antibody against Shh (Sanchez, P., et al. Proc Natl Acad Sci 101, 12561-12566 (2004)) blocked osteoblast differentiation promoted by the Gnas^{-/-} CM (FIG. 2E). Upregulated Shh mRNA and Shh protein were also confirmed in vivo in HO tissues induced by Gnas loss (FIG. 10A).

To determine whether increased Shh plays an essential role in HO, Shh was removed together with Gnas by generating the Shh^{f/f}; Gnas^{f/f} SMPs and gene deletion was induced by Ad-Cre infection (Lewis, P. M., et al. Cell 105, 599-612 (2001)) (FIG. 2F). Loss of Shh led to reduced osteoblast differentiation and Hh signaling (FIGS. 2F and 10B) in vitro. Furthermore, removing one copy of Shh drastically reduced Gnas^{-/-} induced HO in vivo, whereas removing both Shh copies further reduced HO, particularly 3 months after injection, leading to almost complete phenotypic and molecular rescue (FIGS. 2G and 10C). Efficient Shh removal by Ad-Cre injection was confirmed by genomic PCR of the Shh floxed allele and Western blotting of Shh protein (FIG. 10D). The more profound rescue effect of Shh loss observed 3 months post Ad-Cre injection supported the notion that ectopic Shh expression mediates cell non-autonomous effects of Gnas^{-/-} cells during HO expansion, while Gnas^{-/-} cells differentiate into osteoblast cells even in the absence of Shh. In the tissue sections, Osx immunostaining showed that while there were substantial number of Osx⁺; tdTMT⁻ cells in the Gnas^{f/f}; R26^{LSL-tdTMT} mice 3 months after Ad-Cre injection, these cells were drastically reduced in number, but the Osx⁺; tdTMT cells were still present in the Shh^{f/f}; Gnas^{f/f}; R26^{LSL-tdTMT} mice (FIG. 2H). These results together show that Shh secretion by the Gnas^{-/-} SMP cells promoted osteoblast differentiation of the surrounding WT cells.

### Example 3. Gnas Loss Upregulates Shh and Activates Yap Transcription Activities

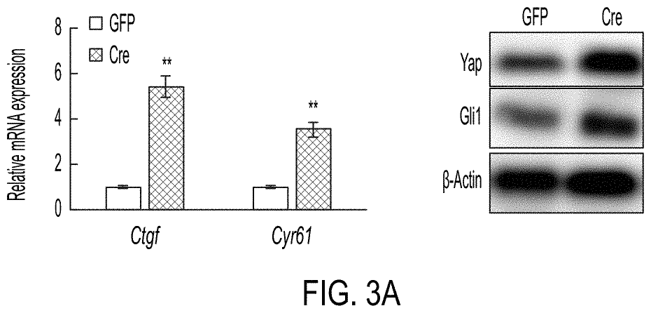

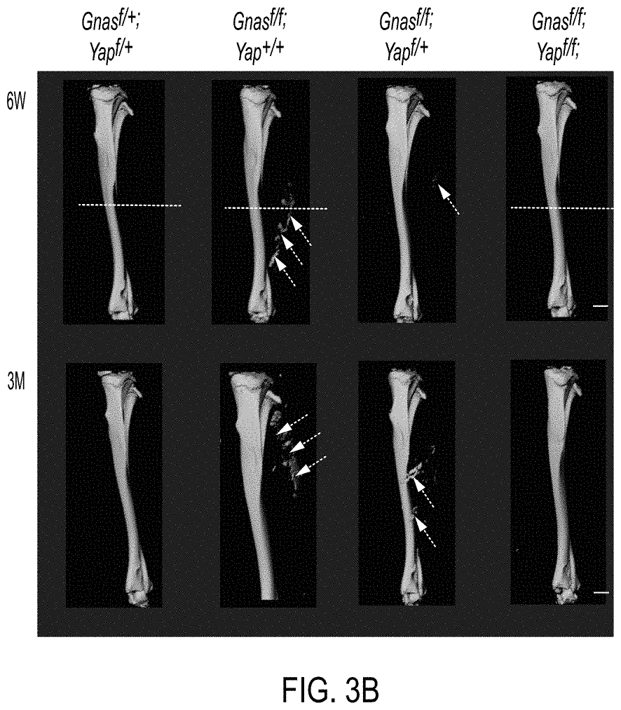

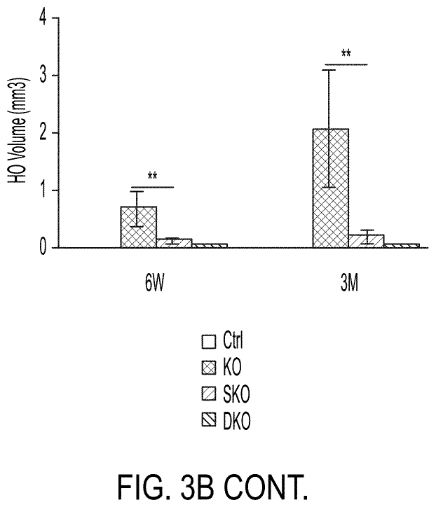

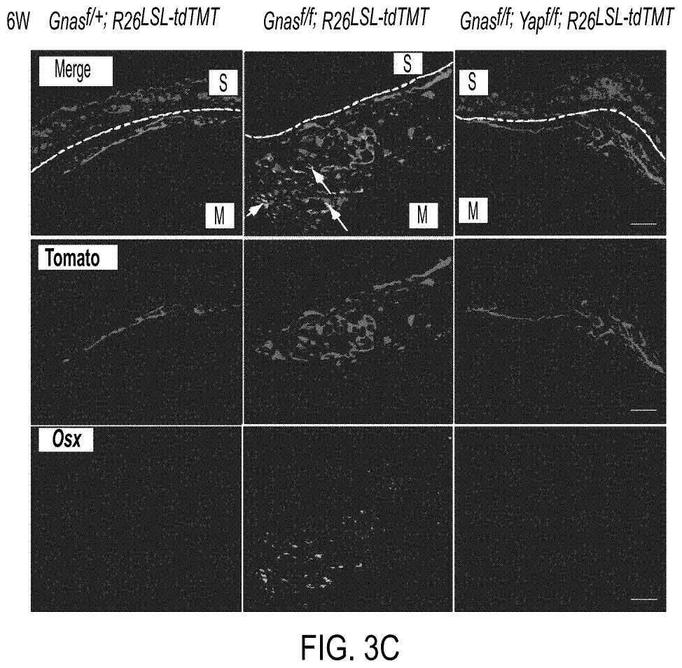

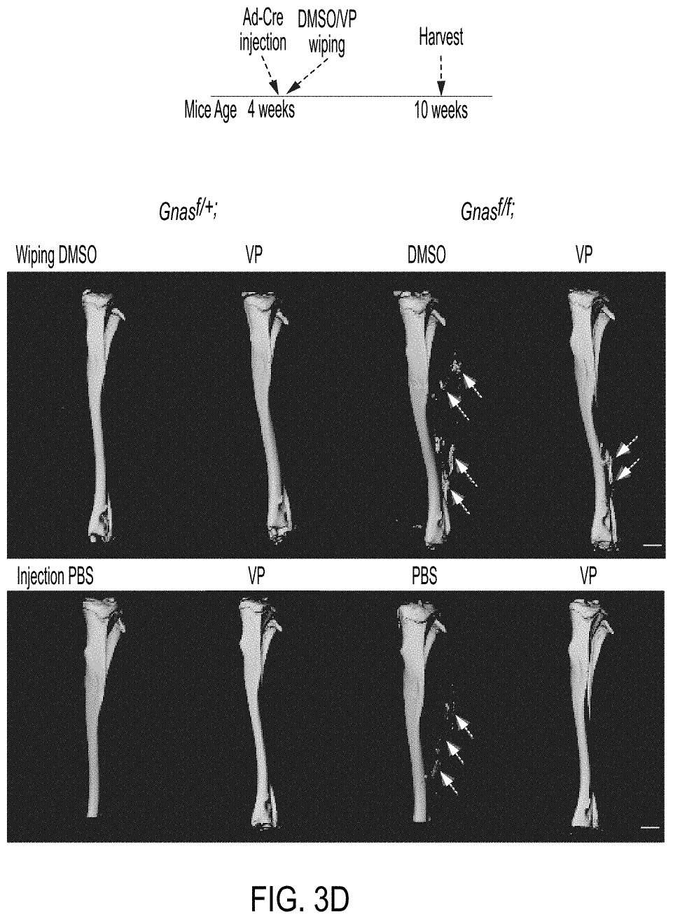

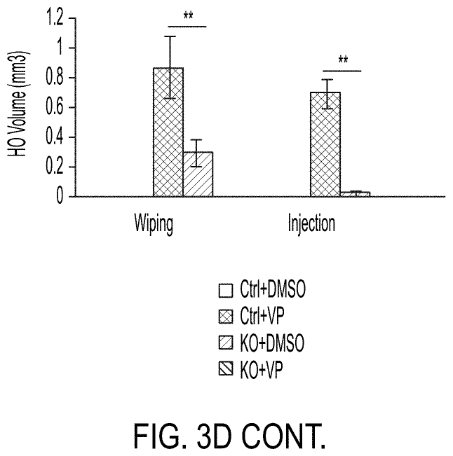

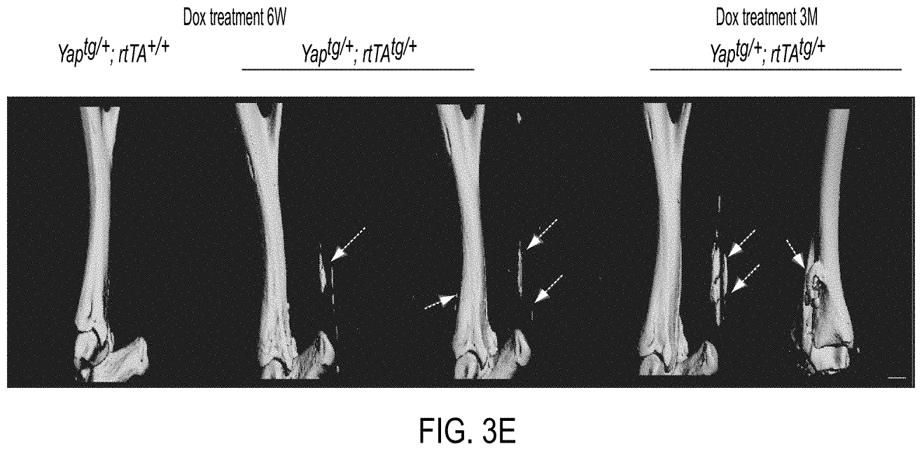

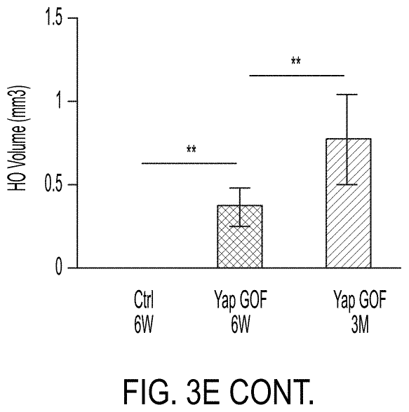

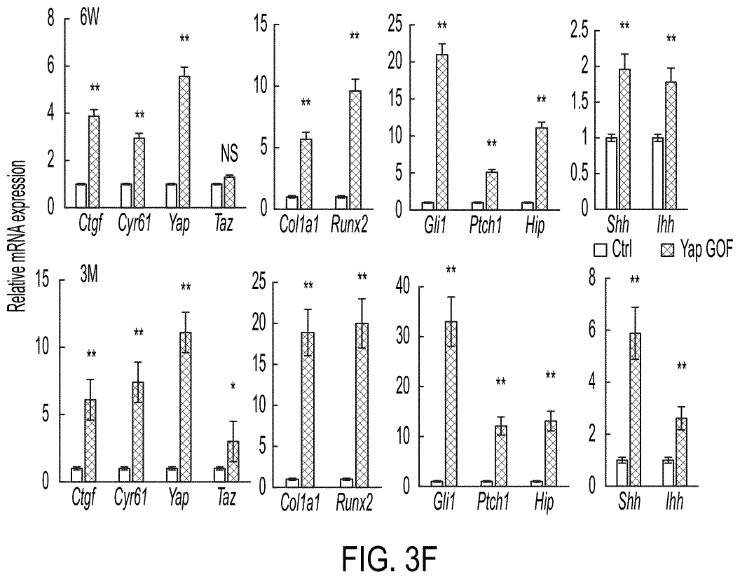

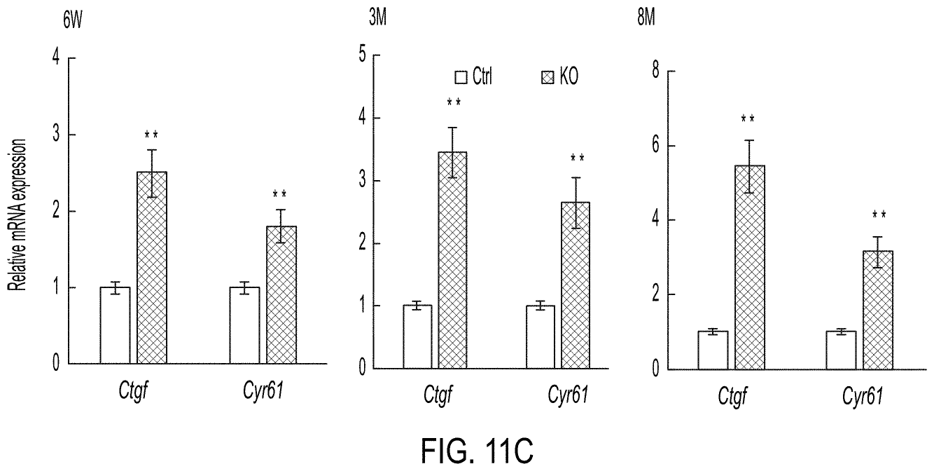

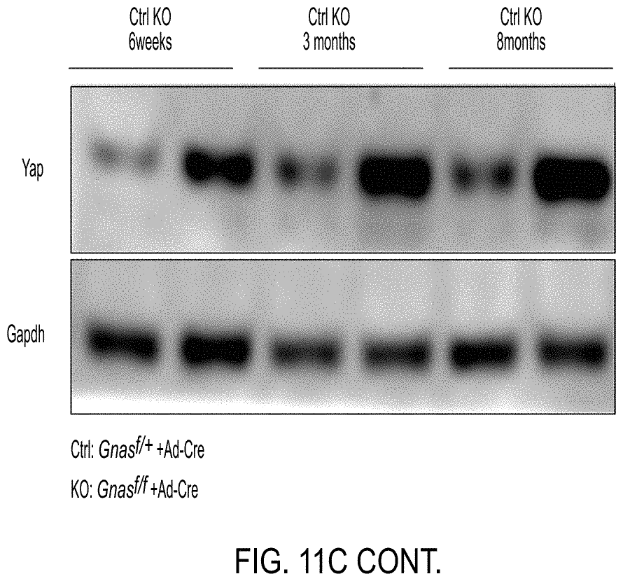

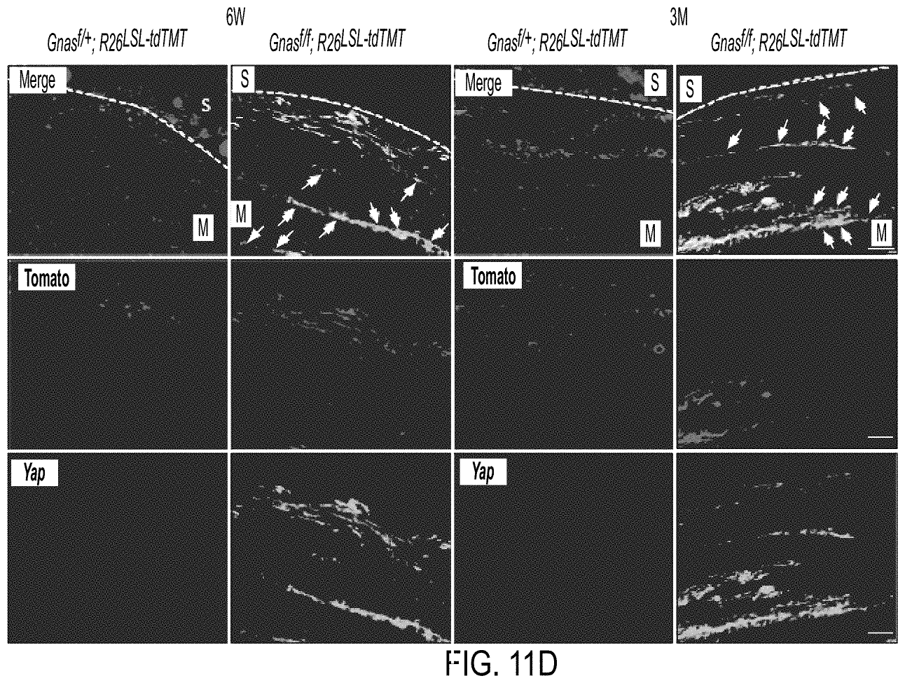

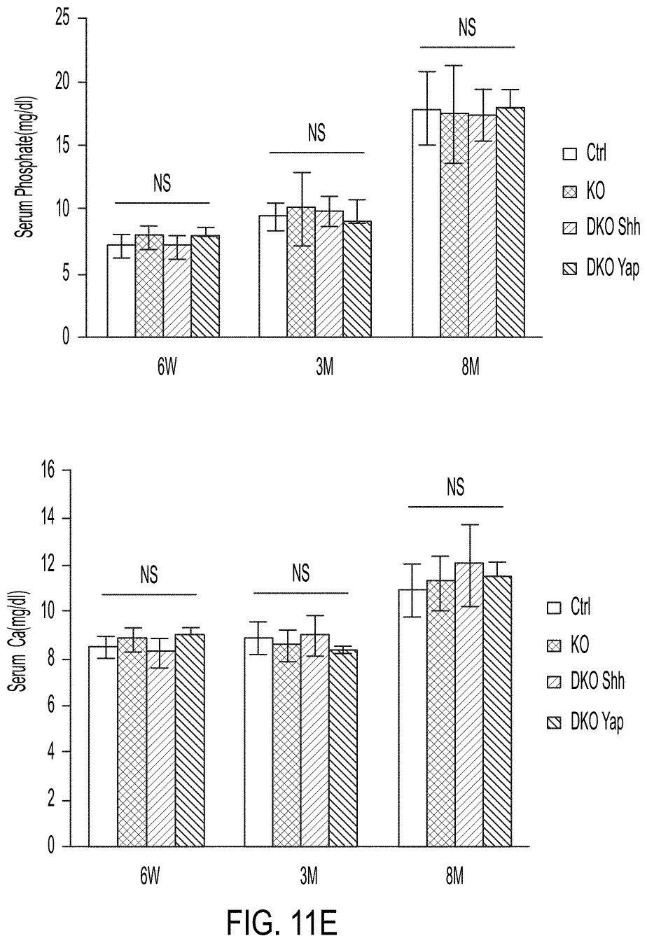

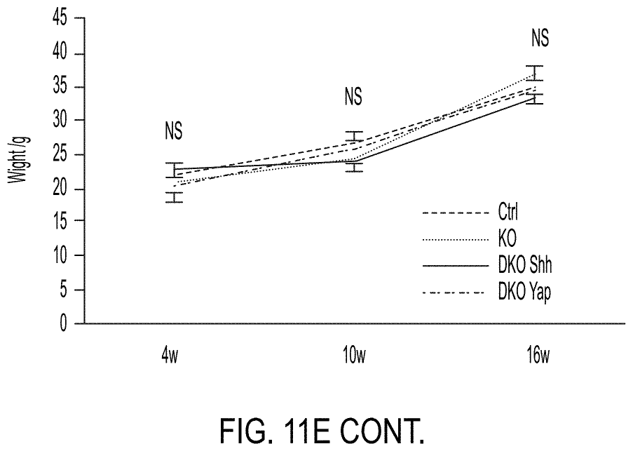

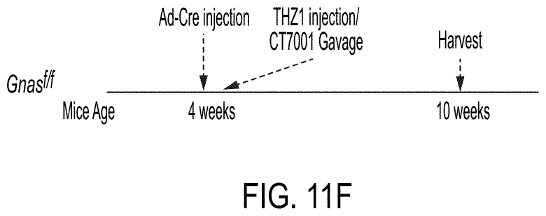

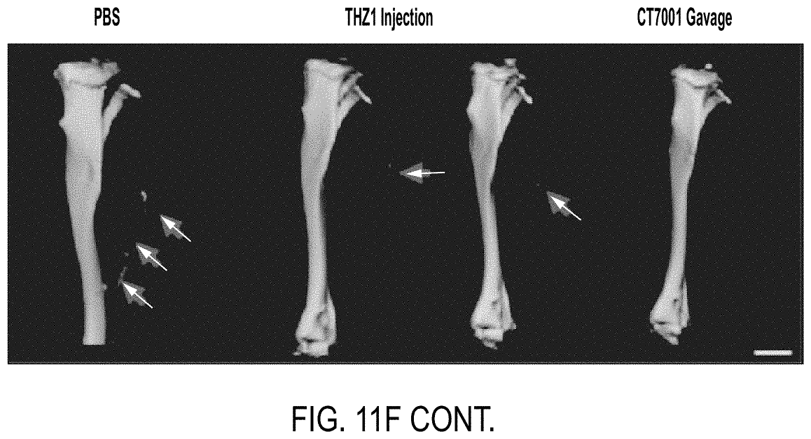

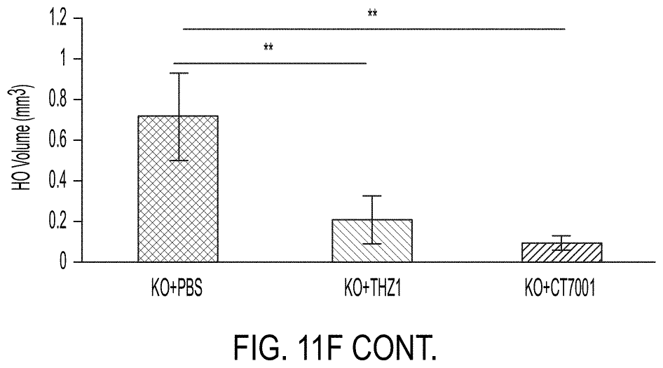

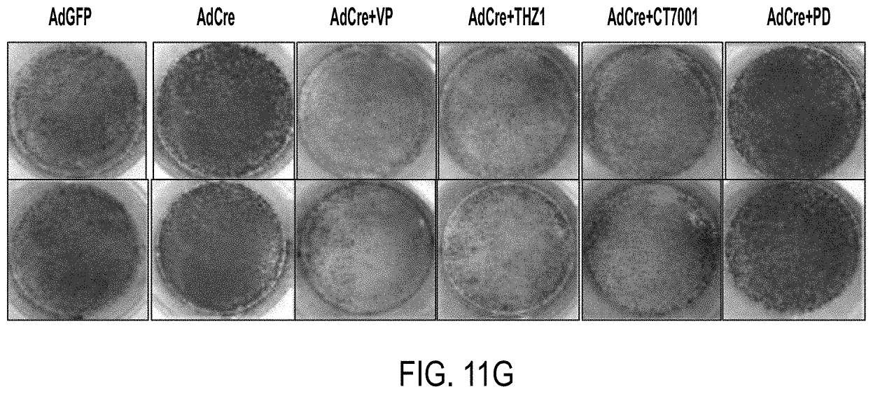

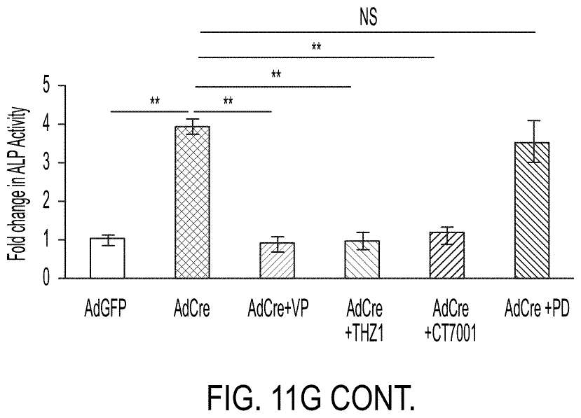

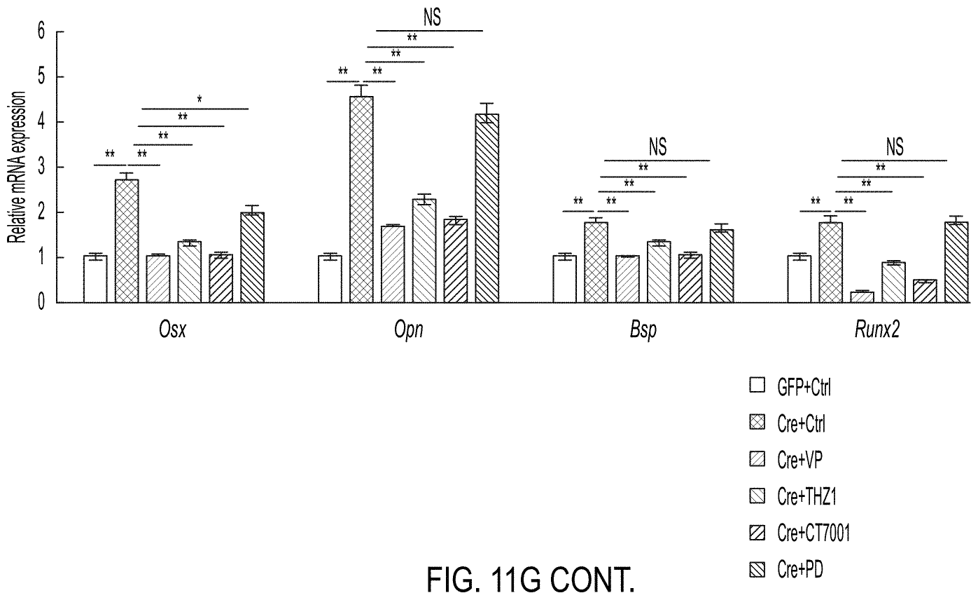

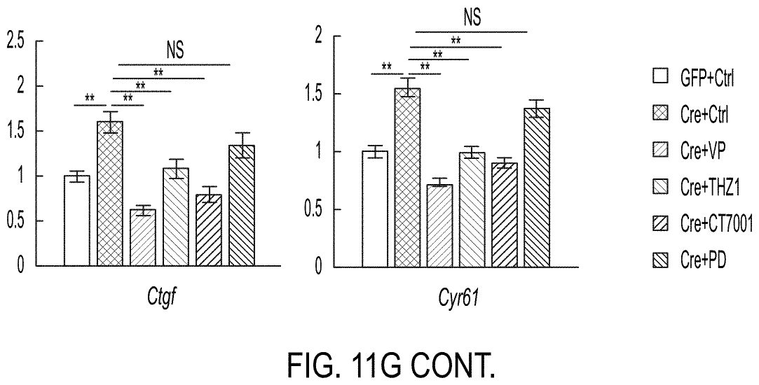

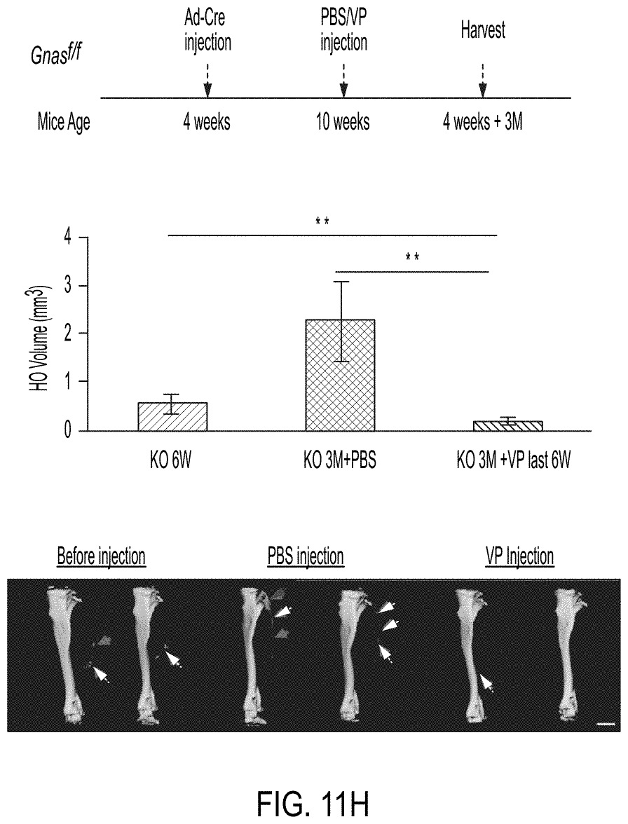

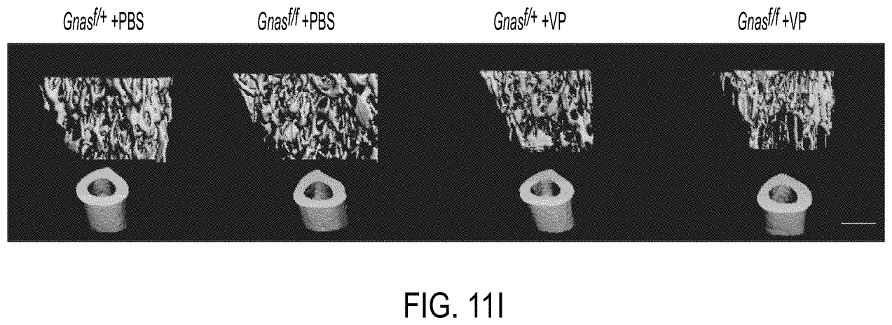

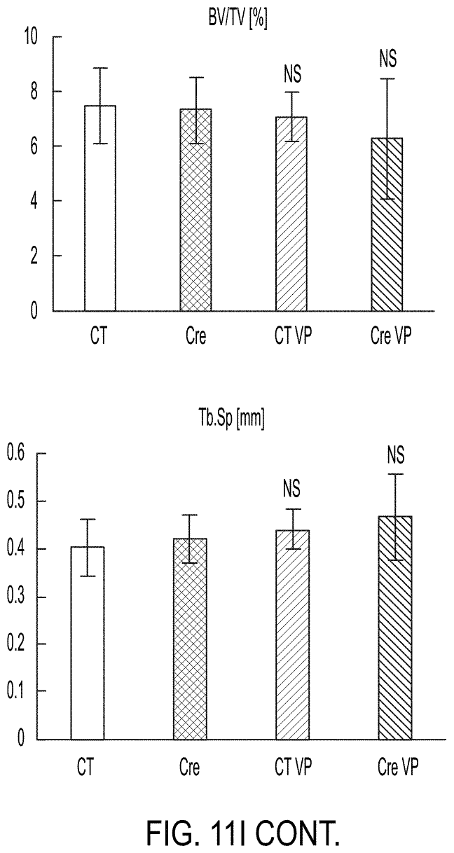

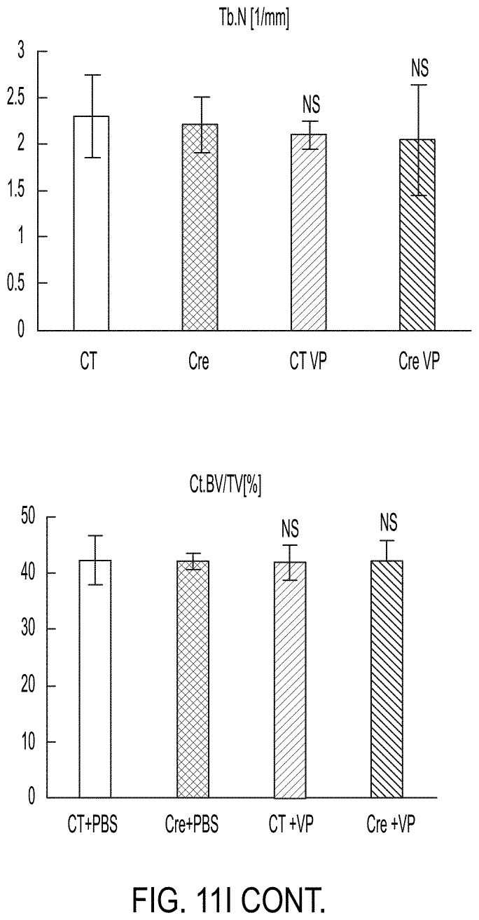

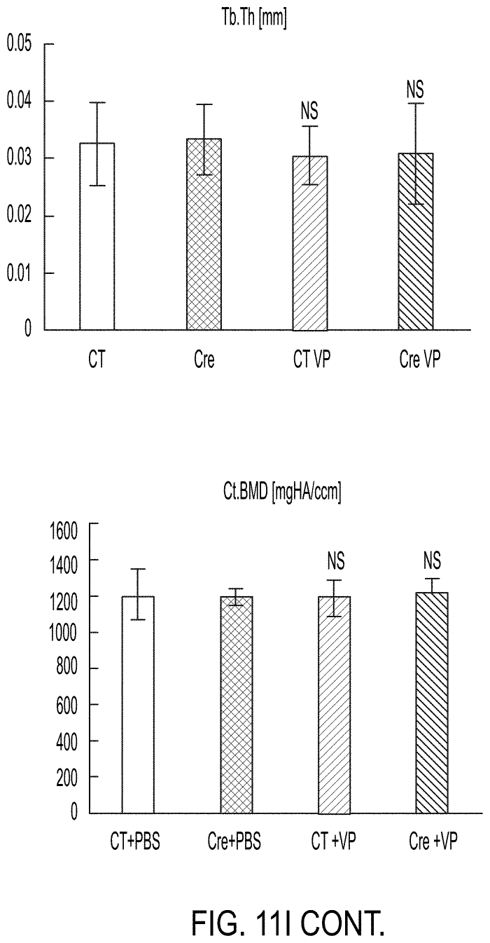

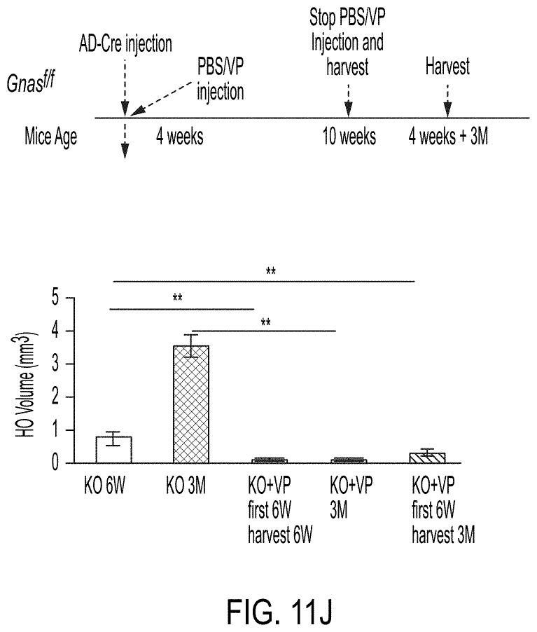

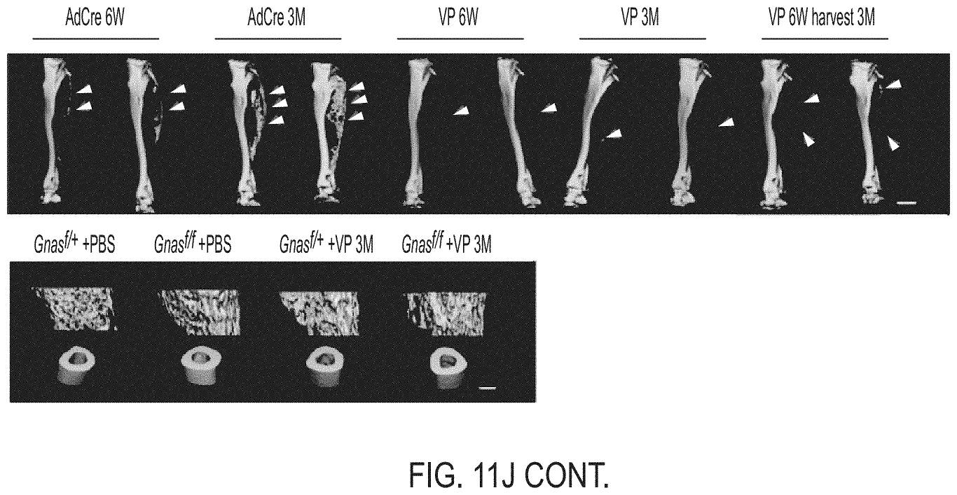

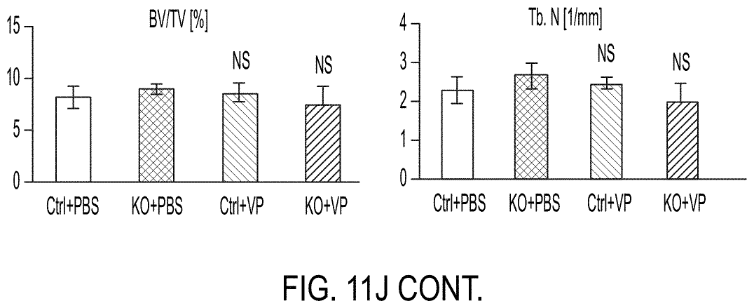

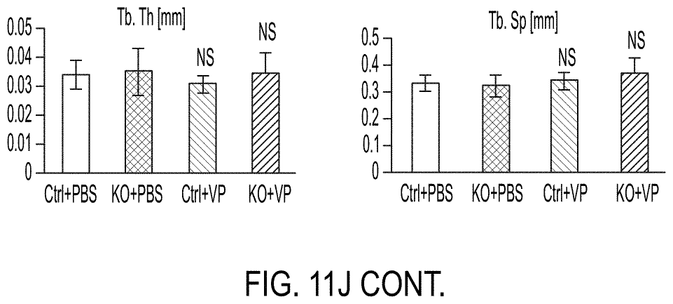

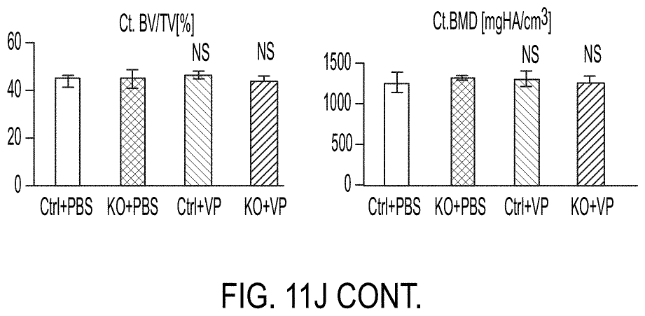

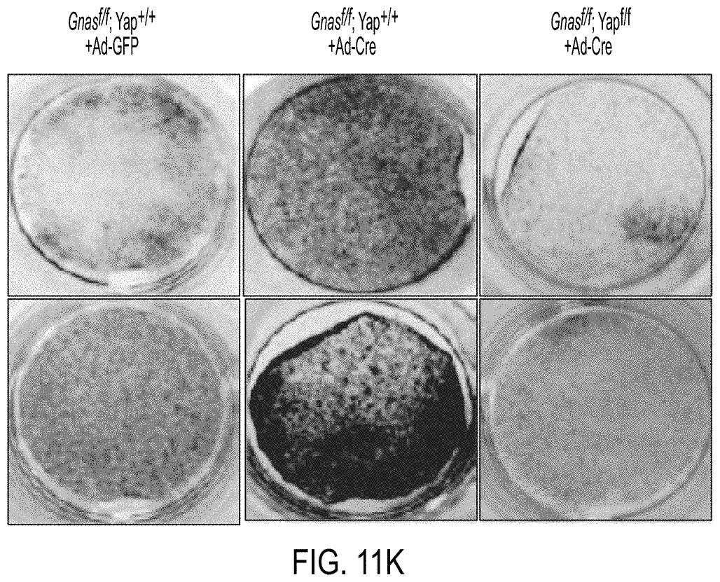

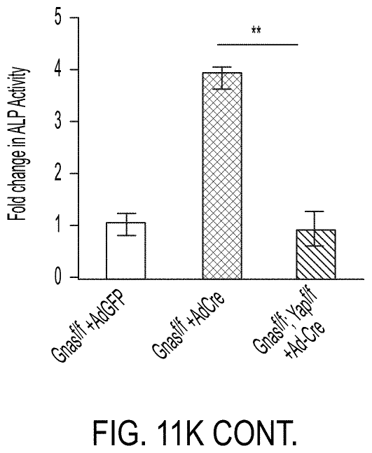

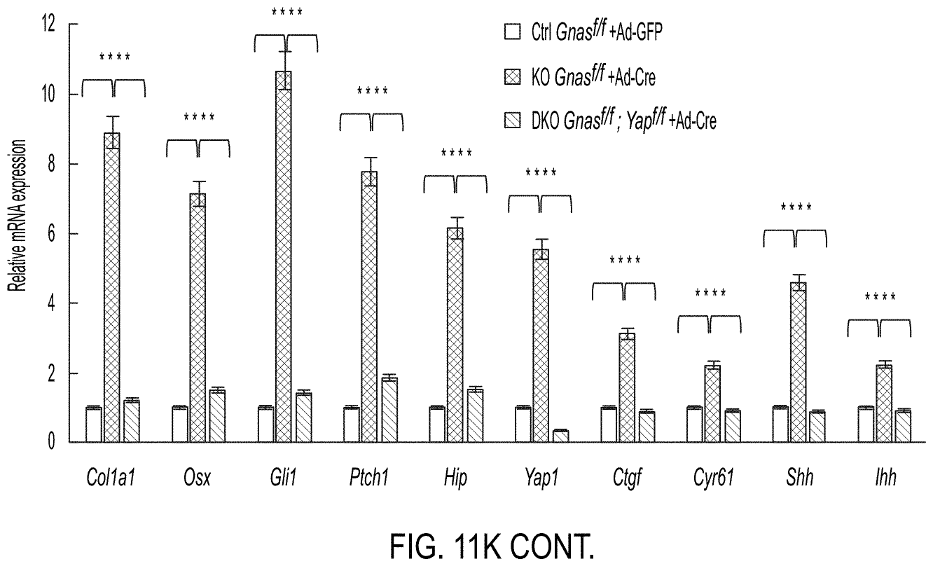

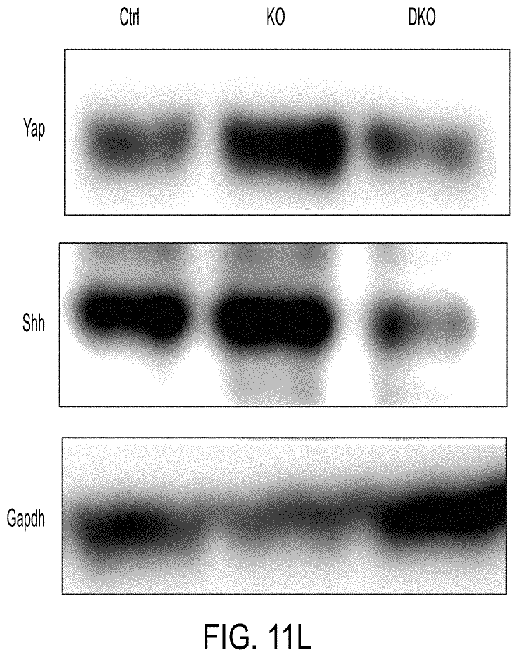

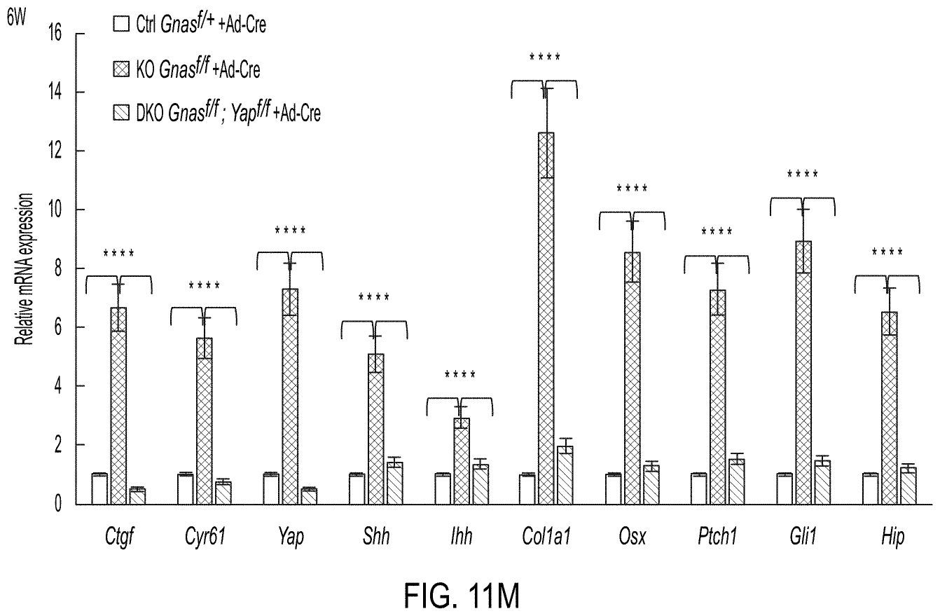

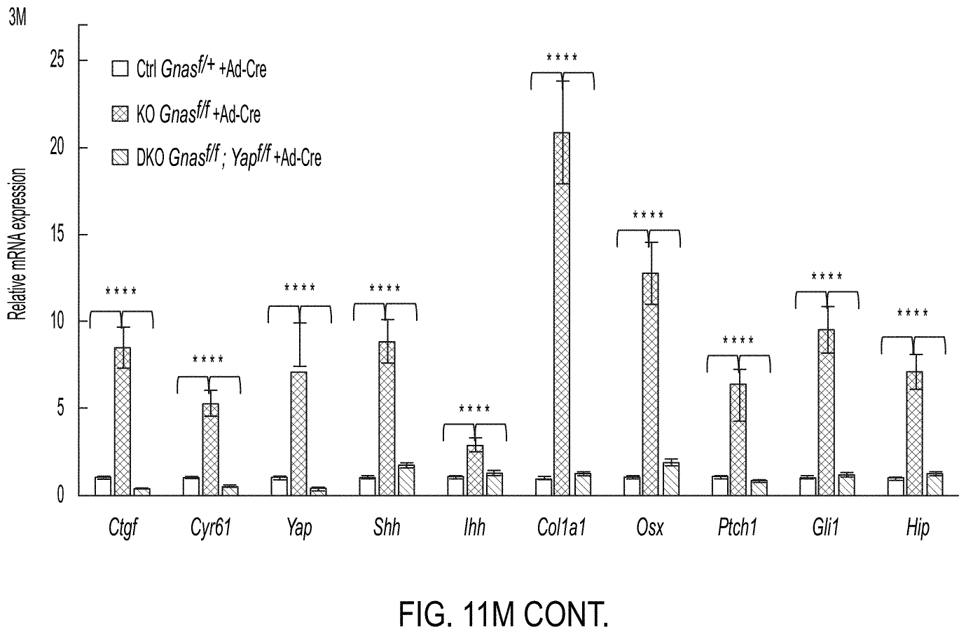

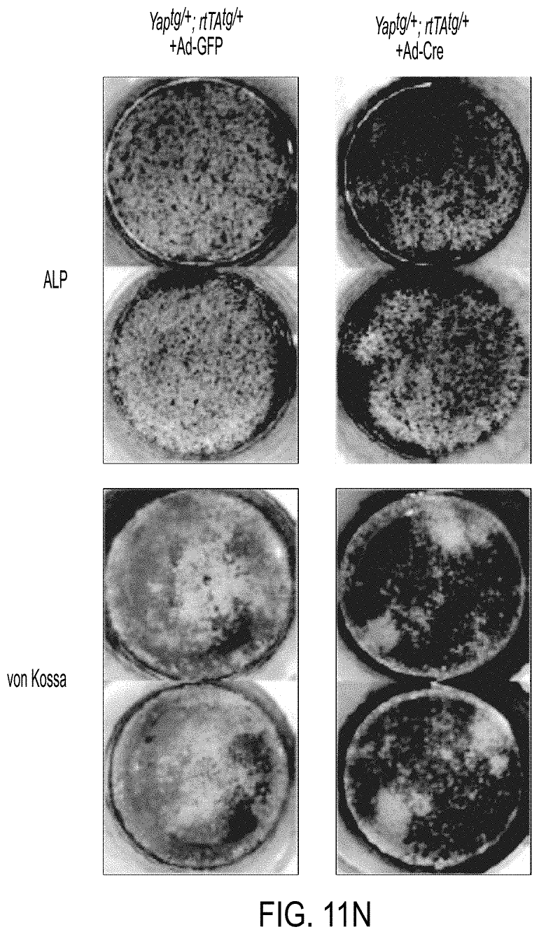

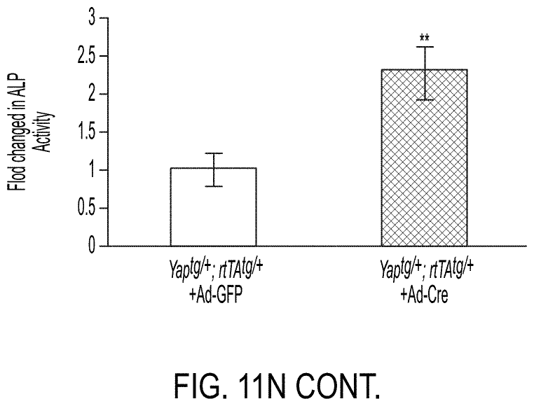

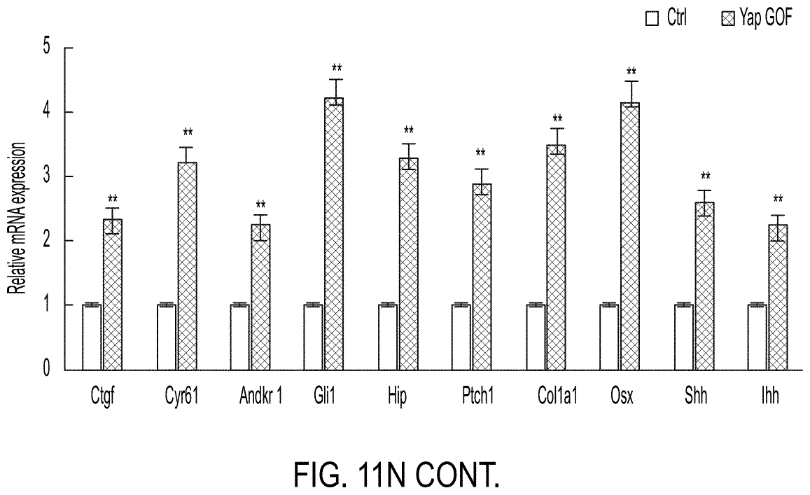

To determine the mechanism underlying ectopic Shh expression, RNA seq experiments were performed for WT and Gnas deficient SMP cells (FIGS. 11A and 11B). The Cyclic adenosine 3′,5′-monophosphate (cAMP) signaling was paradoxically upregulated, possibly due to upregulation of some of the negative regulators of cAMP signaling such as Phosphodiesterase 3a (Pde3a). PKA activity, shown by phosphor-Creb levels, was indeed reduced in the Gnas-deficient SMP cells shown by Western blotting analysis (FIG. 2C). Apart from the altered cAMP signaling, Yes-associated protein (Yap) transcription activity was also increased. Yap is a transcriptional factor in the Hippo signaling pathway that critically regulates cell proliferation, differentiation and survival in development and tumorigenesis (Pan, D. Dev Cell 19, 491-505, (2010); Barron, D. A. & Kagey, J. D. Clin Transl Med 3, 25 (2014); Mo, J. S., Park, H. W. & Guan, K. L. EMBO Rep 15, 642-656 (2014)). Upregulation of Yap activities were further confirmed by quantification of RNA seq data and Western blotting (FIG. 3A). Expression of Yap target genes Ctgf and Cyr61 were increased in the Gnas^{-/-} SMP cells (FIG. 3A). Yap protein levels were also increased along with expected increase of Gli1 protein levels and reduction of PKA activities shown by reduced pCREB levels (FIG. 3A). In HO tissues at 6 weeks, 3 months and 8 months post Ad-Cre injection, it was shown that Yap transcription activities and protein levels were also increased in vivo compared to the control (FIG. 11C). Furthermore, Yap immunoflorescent staining of tissue sections showed that Yap was upregulated in most tdTMT cells in the Gnas^{f/f}; R26^{LSL-tdTMT} mice after Ad-Cre injection (FIG. 11D). These results show that loss of Gnas activated Yap activities both in vitro and in vivo in subcutaneous mesenchymal cells, which are consistent with previous studies in the skin stem cells, where loss of Gnas activated both Gli and Yap activities in the context of basal-cell carcinoma Interestingly, some of the Yap⁺ cells were not tdTMT⁺ and the Yap⁺; tdTMT⁻ cells were increased from 6 weeks to 3 months after Ad-Cre injection (FIG. 11D), suggesting that Yap activation can also be non-cell autonomous.

To test whether Yap is required for HO formation and expansion, Yap was removed together with Gnas by generating the Gnas^{f/f}; Yap^{f/+} and Gnas^{f/f}; Yap^{f/f} mice and Ad-Cre was injected subcutaneously. Indeed, partial loss of Yap drastically reduced HO caused by Gnas loss, while complete Yap loss resulted in more complete HO inhibition at 6 weeks or 3 months after Ad-Cre injection (FIG. 3B). Importantly, HO was not detected in mice with complete Yap loss, while some HO was always present in mice with Shh loss (Compare FIG. 3B with FIG. 2G). Furthermore, immunohistochemistry showed that while many Osx⁺ cells were found in both tdTMT⁺ and tdTMT⁻ fractions of the HO tissue of the Gnas^{f/f}; R26^{LSL-tdTMT} mice, no Osx⁺ cells were found in the Gnas^{f/f}; Yap^{f/f}; R26^{LSL-tdTMT} mice (FIG. 3C). In all of the Gnas, Shh or Yap single or double conditional mutant mice, HO was induced locally by Ad-Cre injection and no adverse effects were detected systemically (FIG. 11E) These results together indicate that Yap is required for ectopic osteoblast differentiation regardless of the Gnas status.

As the results showed that Yap loss resulted in complete HO inhibition, further experiments were performed to test whether HO can be inhibited by a Yap inhibitor Verteporfin (VP) that interferes Yap-Tead binding required for Yap mediated gene expression (Giraud, J., et al. Int J Cancer (2019)). VP was topically applied or IP injected to the Gnas^{f/f} mice immediately after Ad-Cre injection (FIG. 3D), both led to HO reduction, with more effective HO blockage by IP injection of VP (FIG. 3D). In addition, recent studies found that Yap activity is enhanced in the nucleus by CDK7. Inhibition of CDK7 by two distinct inhibitors THZ1 and CT7001 (ICEC0942) (Kwiatkowski, N., et al. Nature 511, 616-620 (2014); Patel, H., et al. Mol Cancer Ther 17, 1156-1166 (2018)) in the Gnas^{f/f} mice after Ad-Cre injection both reduced HO (FIG. 11F). Indeed, osteoblast differentiation was reduced by treatment of Gnas^{-/-} SMP cells in vitro with VP, THZ1 and CT7001, but not Palbociclib (PD-0332991), an inhibitor of CDK4/6 (FIG. 11G).

Furthermore, to determine whether Yap inhibition could lead to reduced HO after its formation, VP was injected into Gnas^{f/f} mice 6 weeks after Ad-Cre injection and we found that HO was obviously reduced 6 weeks later compared to the untreated ones, indicating that Yap inhibitor can reduce already formed HO (FIG. 1111). To determine whether VP treatment may reduce the endogenous bone and therefore cause osteoporosis, the tibia bone mass was analyzed 6 weeks after VP IP injection and no significant change was detected between treated and untreated groups (FIG. 11I), suggesting that Yap inhibition reduced HO, but not the normal bone. To ask whether short inhibition could have long-term protective effects, we stopped the VP treatment after 6 weeks and harvested the samples 3 months after initial Ad-Cre injection (FIG. 11J). Indeed, early and short VP treatment for 6 weeks significantly reduced HO even after VP withdrawal. Furthermore, we found that even after 3 months of non-stop VP treatment, the endogenous bone mass was not reduced (FIG. 11J), suggesting that the bone reduction effects of Yap inhibition is largely confined to HO.

To understand the cellular and molecular mechanism whereby Yap promotes HO, in vitro experiments were first performed using SMP cells cultured under osteogenic conditions. Loss of Yap together with Gnas reduced osteoblast differentiation compared to Gnas loss (FIG. 11K). Importantly, Hh signaling was also reduced by Yap loss and furthermore, Shh expression and Shh protein production were much reduced by Yap loss (FIGS. 11K and 11L). QRT-PCR analysis of the tissues also showed that expression of osteoblast markers, Hh signaling target genes and Shh expression were reduced in the Ad-Cre injected Gnas^{f/f}; Yap^{f/f} mice compared to the similarly treated Gnas^{f/f} mice (FIG. 11M). These results indicate that Yap promotes HO by promoting Shh expression and Hh signaling.

To determine whether Yap activation alone is sufficient to induce HO, expression of a constitutively active, phospho-deficient Yap was induced by subcutaneous Ad-Cre injection of the Cre- and Tet-inducible Yap^{tg/+}; rtTA^{tg/+} mice (Yimlamai, D., et al. Cell 157, 1324-1338 (2014); Ganem, N. J., et al. Cell 158, 833-848 (2014), followed by Doxcycline (Dox) water treatment (FIG. 3E). Indeed, Yap activation alone is sufficient to induce HO and HO size was also progressively increased after Ad-Cre injection (FIGS. 3E and 3F). Further QRT-PCR analysis showed that Yap activation led to increased expression of osteoblast markers, Shh/Ihh expression and upregulated Hh signaling in the SMP cells (FIG. 3F and FIG. 11N). Compared to Gnas loss, Yap activation induced less ectopic osteoblast differentiation and HO was preferentially found in the Achilles tendon, suggesting that though Yap is required for ectopic osteoblast differentiation, Yap needs to work with other transcription factors activated by Gnas loss to efficiently promote osteoblast differentiation. These results demonstrate that Gnas loss activates Yap that is necessary to induce HO and Shh expression. Our results also identify Yap or Shh inhibition as potential therapeutic strategies for HO reduction in POH.

### Example 4. Yap and Shh Form a Positive Feedback Loop

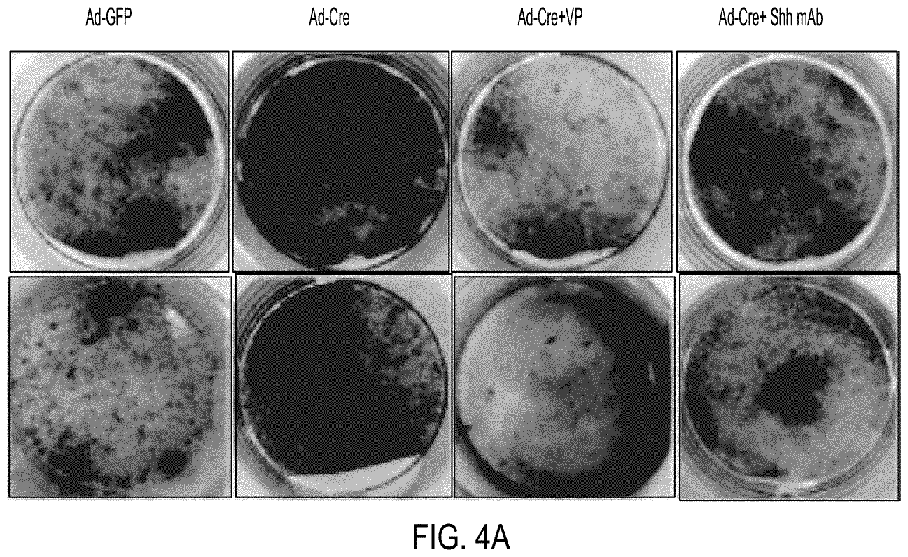

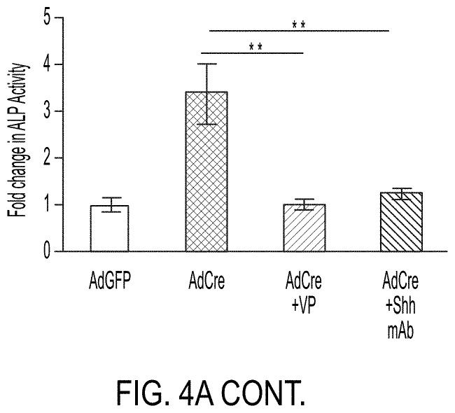

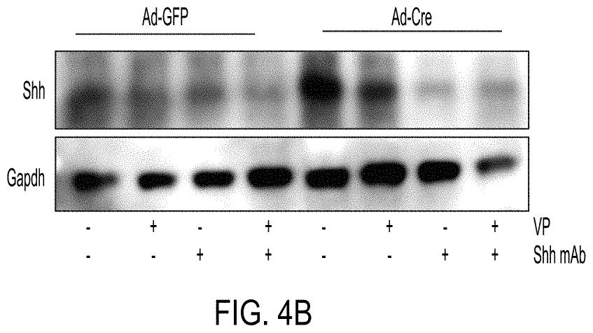

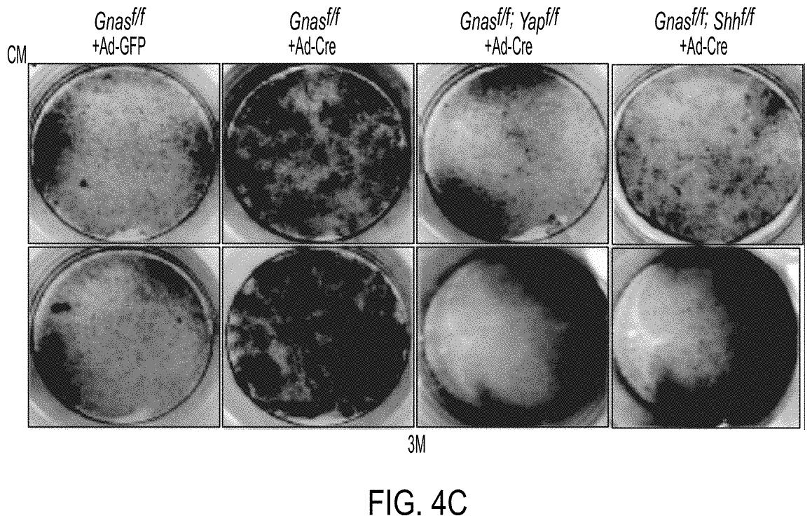

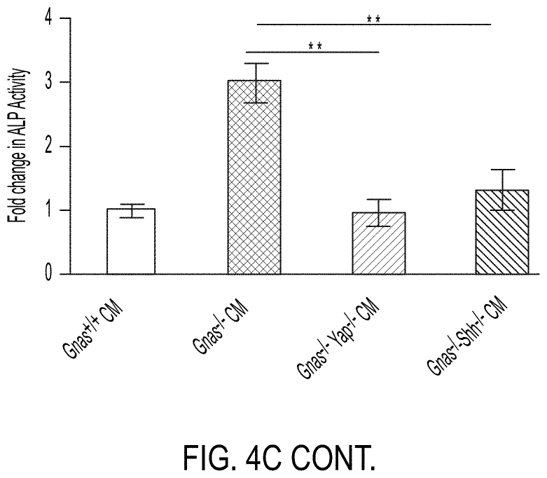

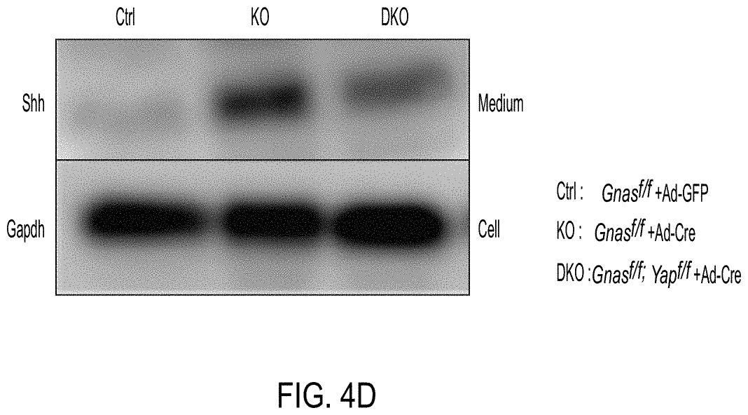

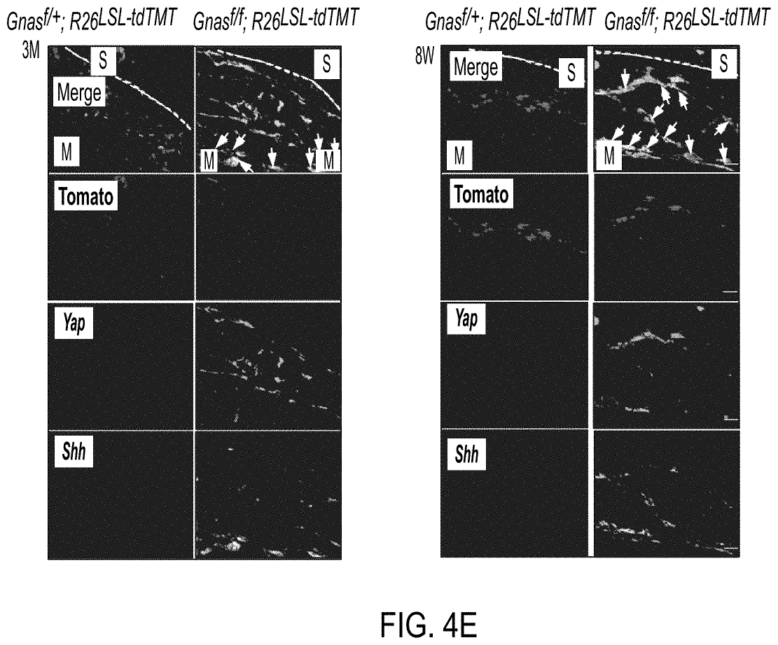

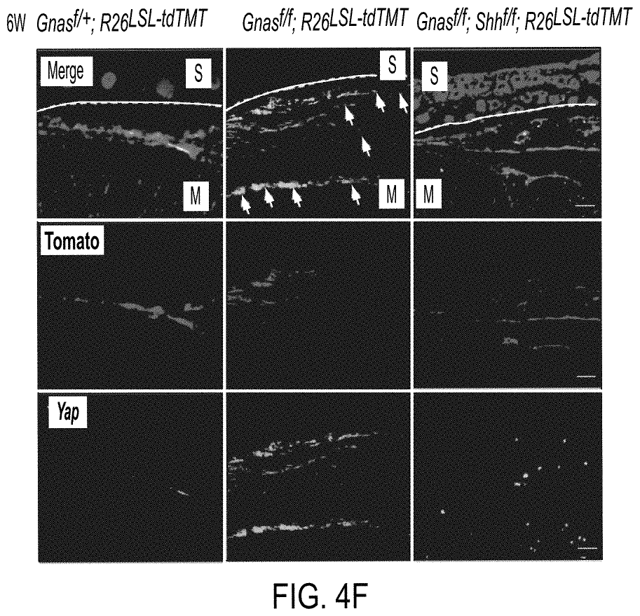

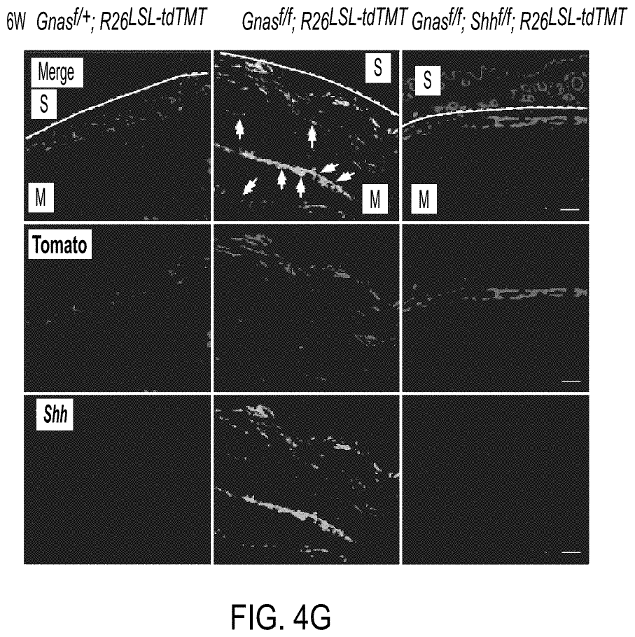

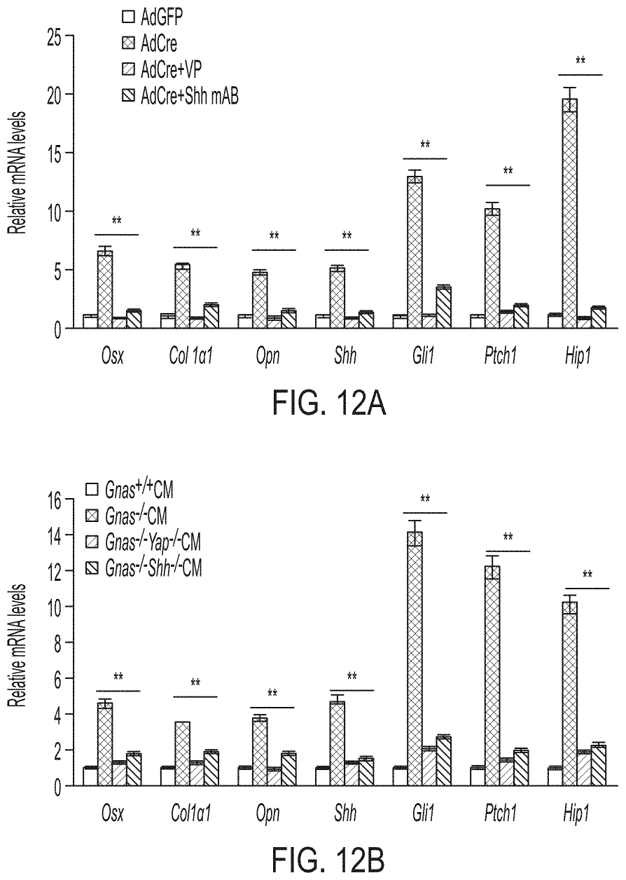

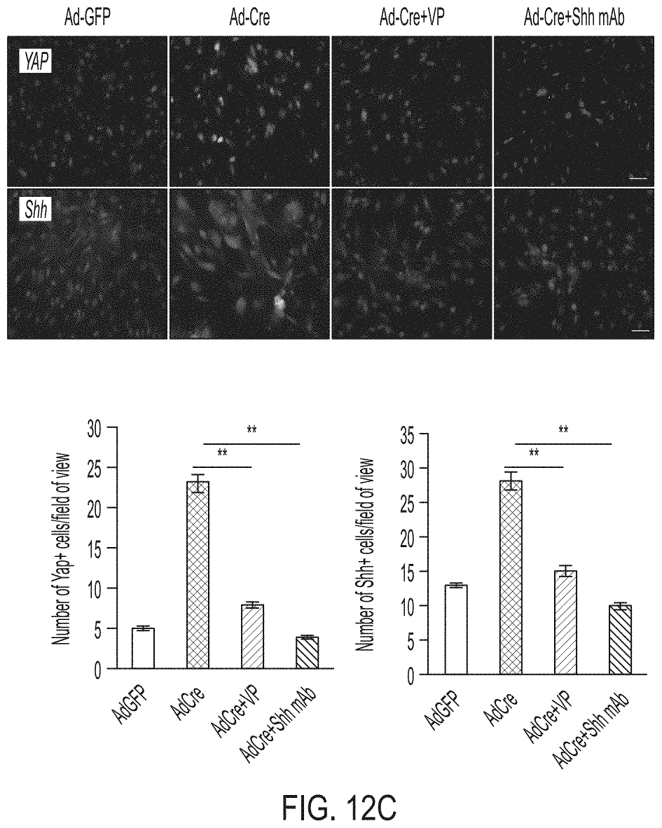

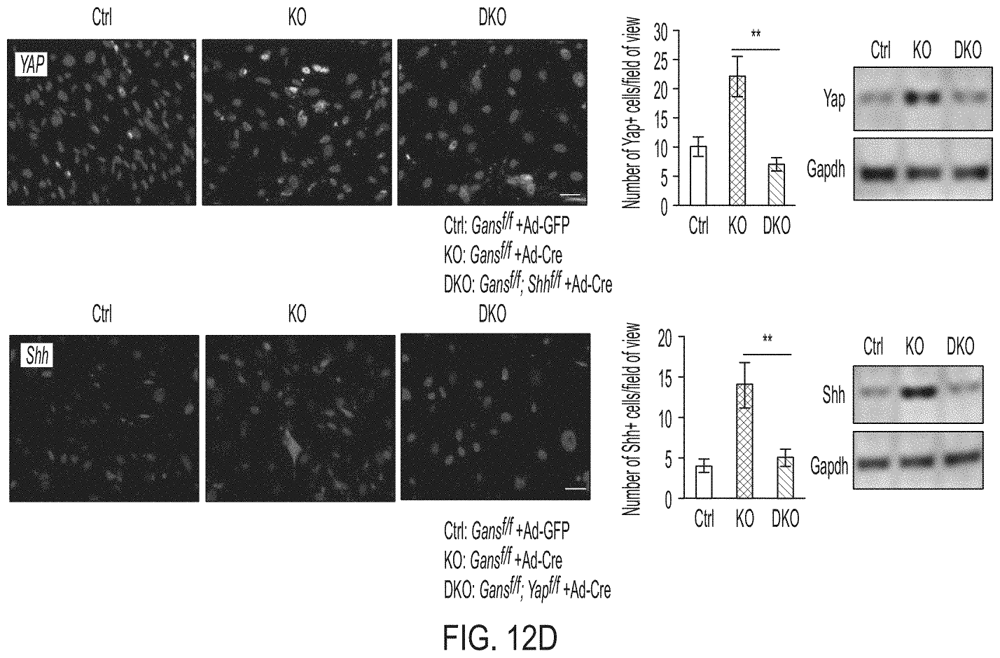

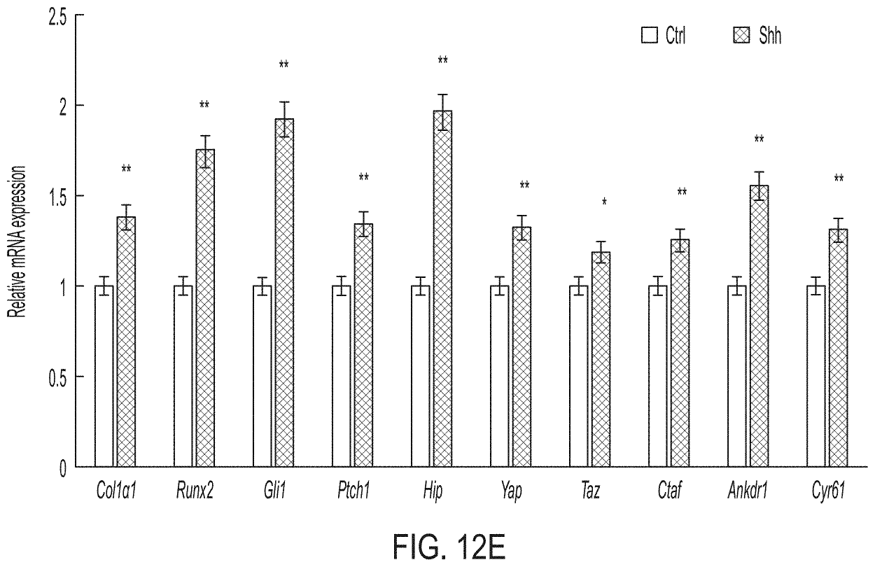

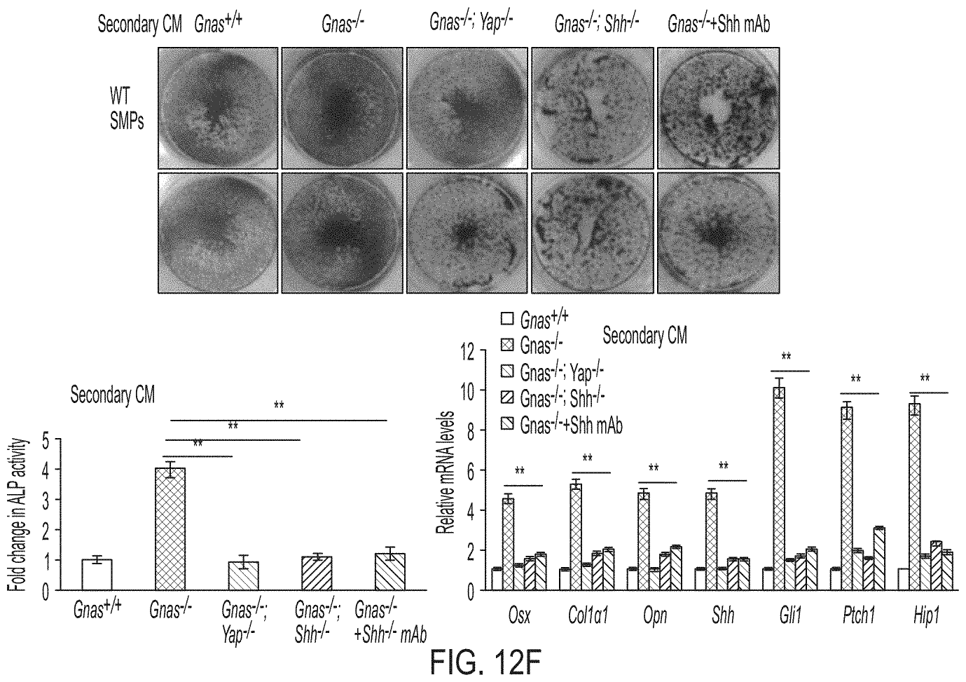

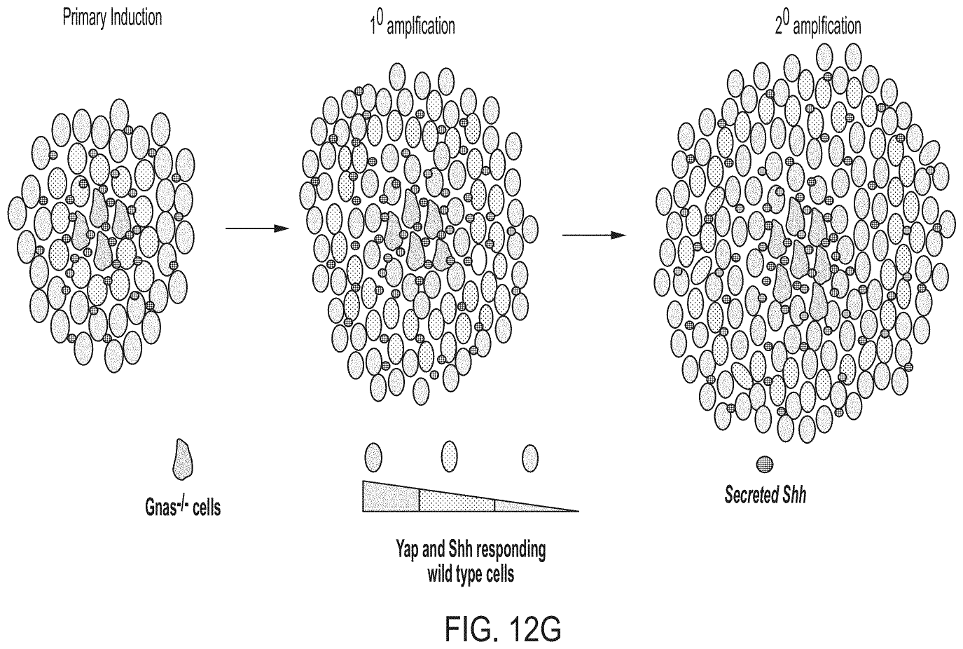

To determine the relationship between Yap activation and Shh expression, both are downstream events of Gnas loss in SMPs, SMPs were first treated with the Yap inhibitor VP or Shh monoclonal blocker for 7 days after Ad-Cre infection. Both treatments decreased osteoblast differentiation and Shh protein levels (FIGS. 4A and 4B, FIG. 12A), suggesting that Yap may activate Shh transcription and there may be a positive feedback loop between Yap and Shh. The results also showed that Yap is required for Shh production in SMPs in vitro and in vivo (FIG. 3F, FIGS. 11K and 11L).

In addition, to test whether Shh is required to activate Yap in SMP cells, the SMPs from Gnas^{f/f}, Gnas^{f/f}; Yap^{f/f} and Gnas^{f/f}; Shh^{f/f} mice were induced by Ad-Cre and the CM was applied to WT SMPs. While the Gnas^{-/-} medium showed elevated osteogenic activities, deletion of either Yap or Shh similarly abolished such activities (FIG. 4C and FIG. 12B). As Yap activates Shh expression, Yap deletion in SMPs indeed reduced Shh protein in the CM (FIG. 4D), indicating that Yap activation is required for progressive HO by recruiting wildtype cells to become osteoblasts via Shh expression and Shh protein secretion (FIG. 4D). Importantly, Shh also activates Yap in SMP cells. In Gnas^{-/-} SMPs, Shh loss reduced Yap levels (FIGS. 12C and 12D). Activation of Yap by Hh signaling has been shown in the skin in the context of basal-cell carcinoma. In SMPs, treatment with recombinant Shh activated expression of Yap target genes, osteoblast markers and Shh-signaling targets (FIG. 12E), indicating that Shh and Yap form a positive feedback loop that promotes osteoblast differentiation of SMPs.

To further determine the relationship between Yap and Shh in vivo, Yap and Shh immunohistochemistry was performed (FIG. 4E). Yap and Shh expression were both upregulated in Gnas^{-/-} cells marked by Tdtomato⁺ after Ad-Cre injection and extended to Tdtomato⁺ wildtype cells (FIG. 4E), suggesting that both Yap and Shh are activated non-cell autonomously, which drive expansion of osteoblast differentiation in progressive HO. Indeed, in the Gnas^{f/f}; Shh^{f/f} mice 6 weeks post Ad-Cre injection, ectopic Yap expression was much reduced (FIG. 4F). In addition, ectopic Shh expression was abolished in the Gnas^{f/f}; Yap^{f/f} mice 6 weeks after Ad-Cre injection (FIG. 4G), demonstrating that Yap and Shh regulate each other.

In addition, while loss of Yap reduced Shh expression in Gnas^{-/-} SMPs (FIG. 11L), loss of Shh in Gnas^{-/-} SMPs also resulted in reduced Yap levels (FIGS. 12C and 12D). Furthermore, when the SMP cells were treated with Shh recombination protein, osteoblast markers as well as target genes of both Shh and Yap were upregulated in expression (FIG. 12E). To further test whether WT cells with Yap activation will express and secrete Shh to induce osteoblast differentiation of more wild type cells, thereby progressively expand the ectopically formed bone, CM from the WT SMPs that had been induced by the CM from the Gnas^{-/-} cells was applied to WT SMPs, and elevated osteoblast induction ability was also observed (FIG. 12F) Taken together, these data indicate that the Gnas^{-/-} cells progressively recruited wild type cells to become osteoblasts by activating Yap and Shh expression. Secreted Shh signals to the nearby WT cells to induce Yap activation, ectopic Shh expression and osteoblast differentiation and a new cycle starts. Therefore, Yap and Shh form a self-amplifying positive feedback loop in wild type cells to promote progressive HO (FIG. 12G).

### Example 5. Yap Directly Activates Shh to Promote HO

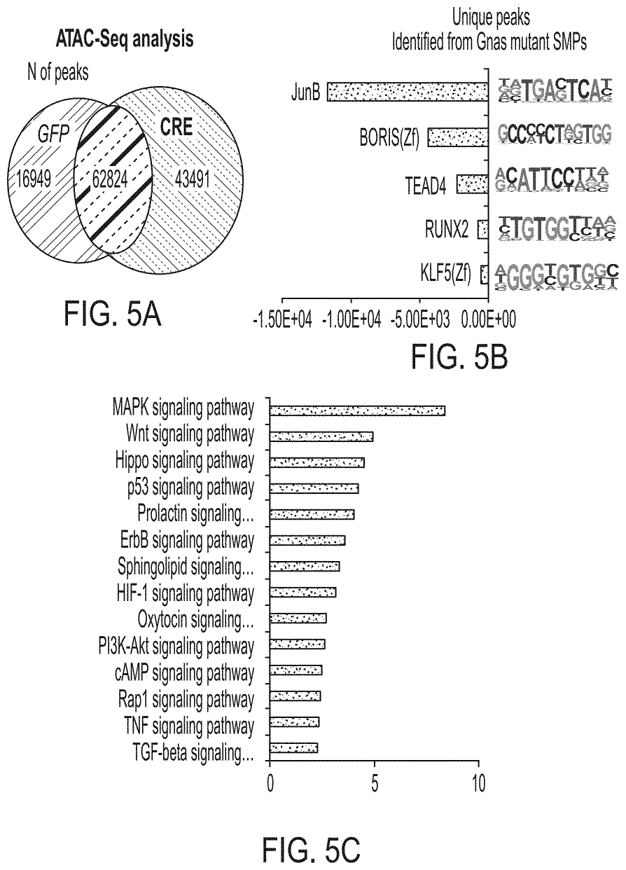

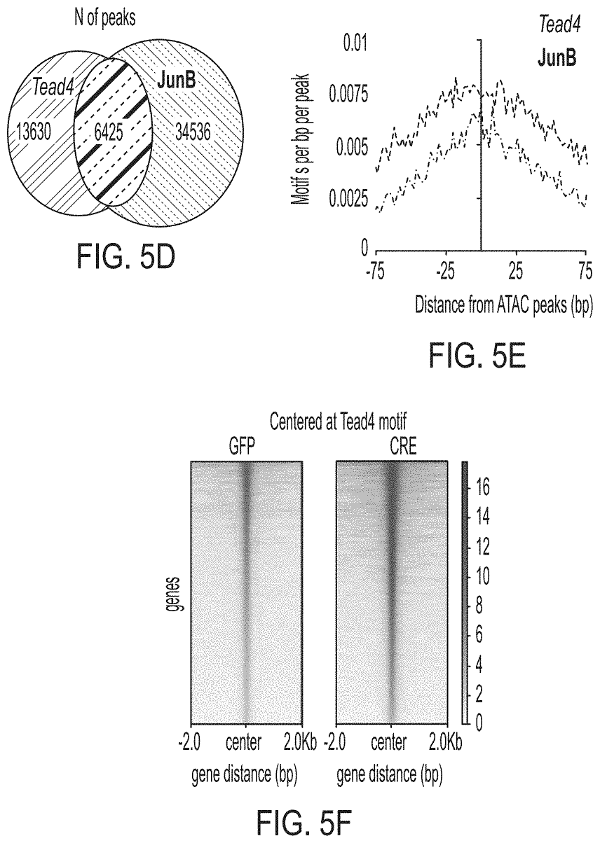

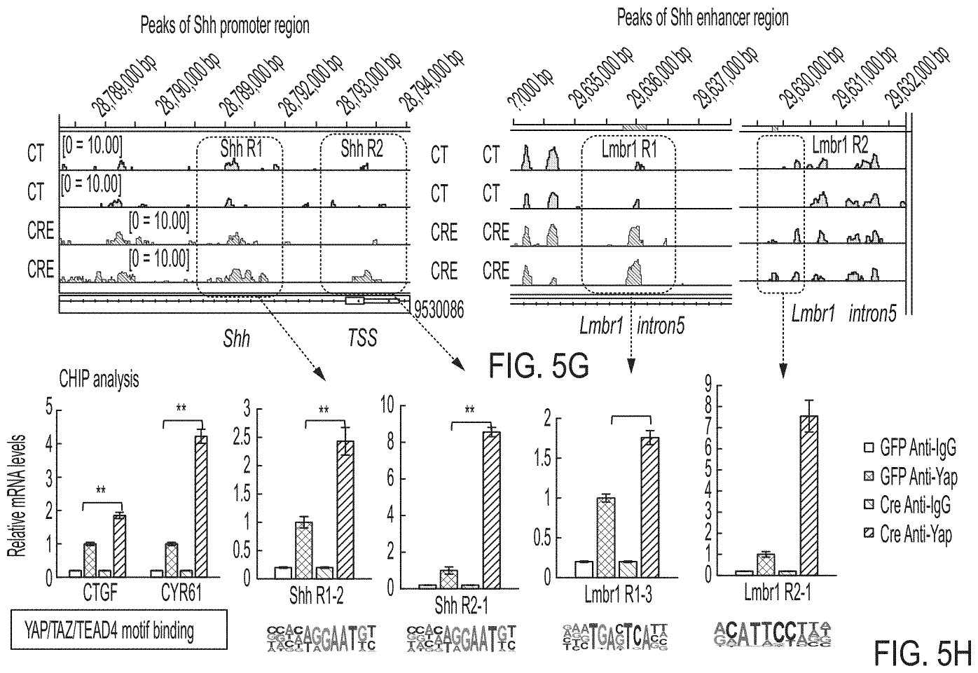

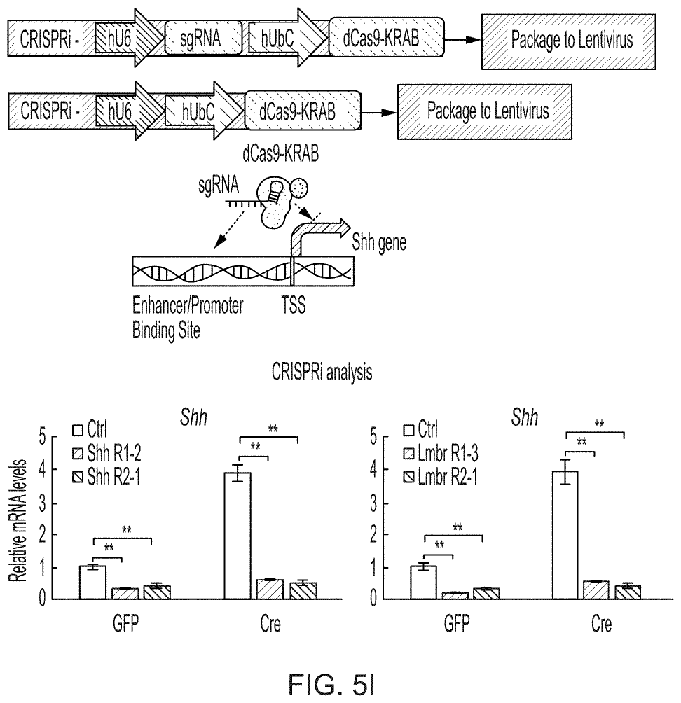

Since ectopic Shh expression was regulated by Yap, alteration in gene expression was further investigated in the Gnas^{-/-} mutant SMP cells. Chromatin remodeling is known to occur during cell fate change. Thus, to understand the molecular mechanism underlying HO induction at a genome-wide gene expression level, Assay for Transposase-Accessible Chromatin using sequencing (ATAC-seq) was performed to determine chromatin accessibility in WT and Gnas^{-/-} SMP cells. The number of ATAC seq peaks were increased in the Gnas^{-/-} SMP cells, indicating that gene transcription is likely more active in the absence of Gnas, consistent with activated Hh signaling and Yap activities in the Gnas^{-/-} SMP cells (FIG. 5A).

To understand changes of transcriptional regulation in the Gnas^{-/-} SMP cells, unbiased de novo motif discovery was performed with the 43491 unique peaks by HOMER software and the top 5 enriched transcription factor (TF) binding motifs were found to be binding sites of JunB, Bris or CCCTC-Binding Factor Like (CTCFL), Tead4, Runx2, and Klf5 (FIG. 5B). Enriched CTCFL binding motif indicates chromatin remodeling in the Gnas^{-/-} SMP cells, consistent with the observed cell fate change, and increased Runx2 binding motifs confirmed osteoblastic differentiation of the Gnas^{-/-} SMP cells. As Yap/Tead and AP1 (JunB) are known to coordinate in gene regulation in different tumor cells, enriched JunB and Tead4 binding motifs suggest Hippo and MAPK signaling may play important roles in osteoblastic differentiation. Indeed, KEGG pathway mapping of the genes with transcriptional start site (TSS) present in the unique ATAC peaks of the Gnas^{-/-} SMP cells showed both Hippo and MAPK signaling pathways were highly up-regulated (FIG. 5C).

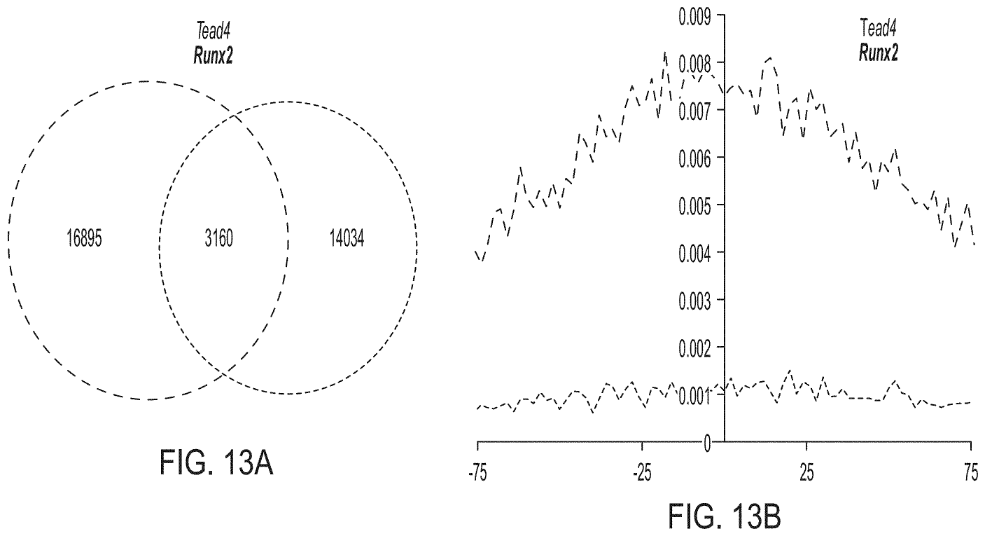

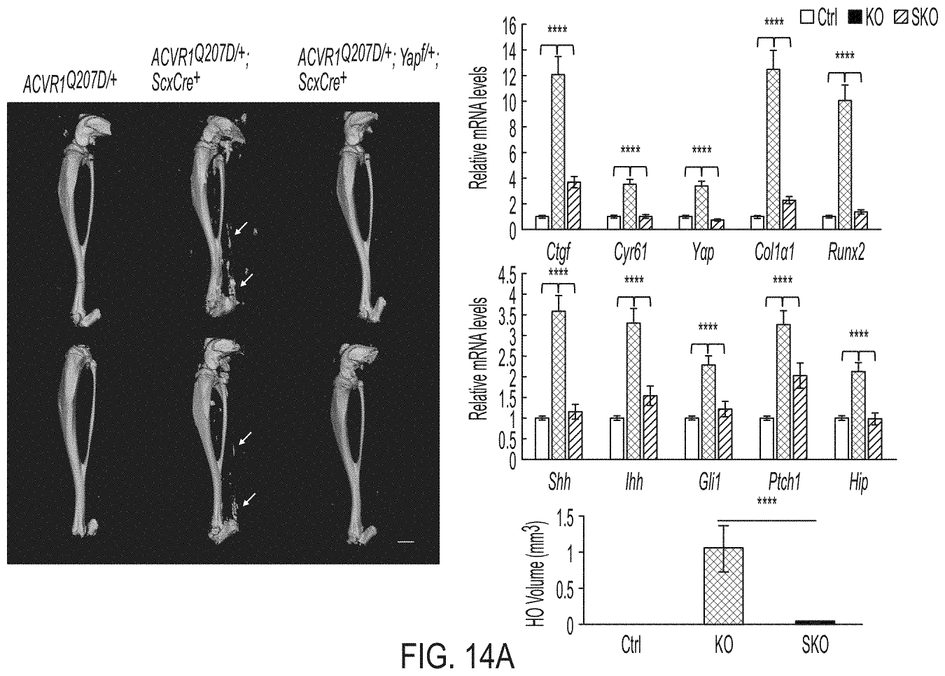

To test whether Tead4 and JunB coordinate in gene regulation, all identified putative Tead4 and JunB binding peaks were separated into unique and overlapping sets, ˜30% of Tead4 binding peaks also contain JunB binding motifs (FIG. 5D). In addition, high co-occurrence of Tead4 and JunB binding motifs was observed in the unique ATAC peaks of the Gnas^{-/-} SMP cells (FIG. 5E). However, although 15% of the Tead4 binding peaks contain Runx2 binding sites, the co-occurrence of Runx2 and Tead4 was low (FIGS. 13A and 13B). These data suggest that in the Gnas^{-/-} SMP cells, Yap/Tead also coordinate with AP1 in transcription regulation to promote osteoblastic differentiation.

Lastly, to determine Yap/Tead4 mediated gene regulation, it was further investigated whether the chromatin accessibility around the Tead4 binding sites were increased in the Gnas^{-/-} SMP cells. The Tead4 motifs were analyzed in all ATAC peaks and it was found that ATAC seq signal intensities were higher around the Tead4 motifs in the Gnas^{-/-} SMP cells (FIG. 5F). Taken together, the ATAC seq data analyses confirmed activation of Yap transcription in the Gnas^{-/-} SMP cells.

As Shh expression was increased in HO, their expression regulation was investigated. The results showed that ATAC seq signal intensities were strongly enhanced in the Shh promoter and enhancer (Lmbr1 intron 5 region (Lettice, L. A., et al. Hum Mol Genet 12, 1725-1735 (2003); Sagai, T., et al. Development 132, 797-803 (2005); Potuijt, J. W. P., et al. Genet Med 20, 1405-1413 (2018)) regions, indicating the chromatin accessibility in the Shh locus was higher in the Gnas^{-/-} SMP cells compared to control cells (FIG. 5G).

To test whether Shh is a direct transcriptional target of Yap, ATAC seq results in Shh promoter and enhancer were analyzed. Several Tead4 binding sites were identified in each region, which were confirmed by CHIP-PCR analysis in SMP cells (FIG. 51I).

To further determine whether the identified Tead4 binding sites are required for Shh expression, the Tead4 sites were blocked in the Shh promoter/enhancer regions by placing mutant Cas9 protein to the Tead4 binding sites with respective guide RNAs (Yeo, N.C., et al. Nat Methods 15, 611-616 (2018); Thakore, P. I., et al. Nat Methods 12, 1143-1149 (2015)). The SMP cells were infected with a lentivirus encoding a dead-Cas9-KRAB fusion protein (dCas9-KRAB) plus guide RNAs targeting each Tead4 binding sites. Targeting dCas9-KRAB to putative enhancers or promoters recruits the H3K9 methyltransferase SETDB1, hence increasing local H3K9me3 levels and repressing target genes (Thakore, P. I., et al. Nat Methods 12, 1143-1149 (2015)). Indeed, occupation of Tead4 sites by dCas9-KRAB in the Shh promotor/enhancer regions significantly reduced Shh expression of Shh (FIG. 5I). Taken together, these data demonstrate that Shh is a direct transcriptional target of Yap.

### Example 6. Yap and Shh are Activated in and Required for HO in FOP Mouse Models

HO in POH is governed by intramembranous ossification, while HO in FOP undergoes endochondral ossification, in which Hh signaling is required for osteoblast differentiation in development (Rodda, S. J. & McMahon, A. P. Development 133, 3231-3244 (2006)). It was then investigated whether the Yap-Shh positive feedback loop also controls HO in FOP.

FOP is caused by mutations in ACVR1 (also known as ALK2) that make it constitutively active (Shore, E. M., et al. Nat Genet 38, 525-527 (2006)). A transgenic mouse line that allows conditionally inducible expression of an artificial, constitutively active variant of the type I BMP receptor (caALK2, or ACVR1^{Q207D}) has been established (Fukuda, T., et al. Genesis 44, 159-167 (2006)). Induction of caACVR1/ACVR1^{Q207D} expression in adult mice has proven valuable for HO studies (Bagarova, J., et al. Mol Cell Biol 33, 2413-2424 (2013)) and specifically, caACVR1/ACVR1^{Q207D} expression driven by the tendon-specific Scx-Cre causes spontaneous HO (Agarwal, S., et al. Stem Cells 35, 705-710 (2017); Dey, D., et al. Sci Transl Med 8, 366ra163 (2016)). Interestingly, Dey et al. reported that heterotopic cartilage, but not bone, was derived from Scx⁺ lineage cells (FIG. 2 in Dey, D., et al. Sci Transl Med 8, 366ra163 (2016)) suggesting that HO in this model is formed cell non-autonomously.

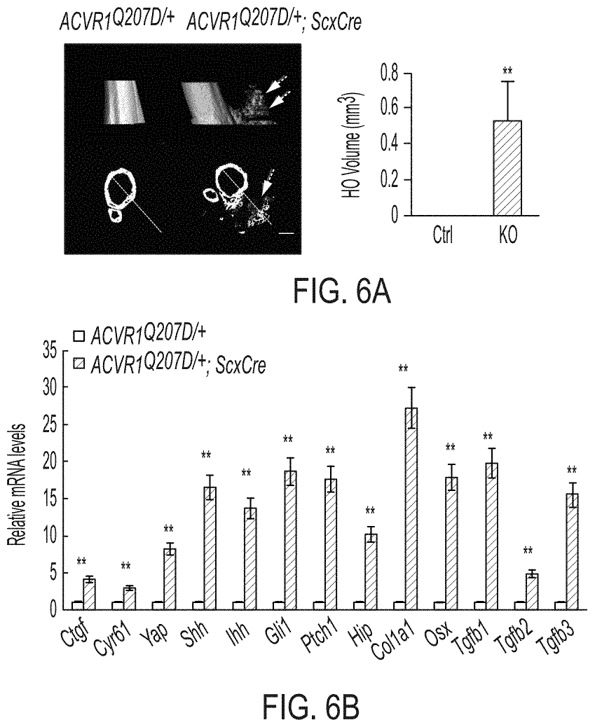

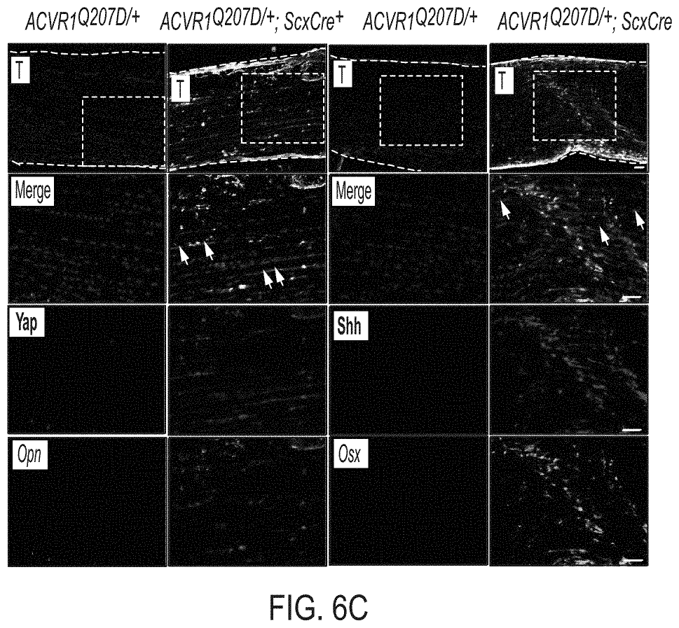

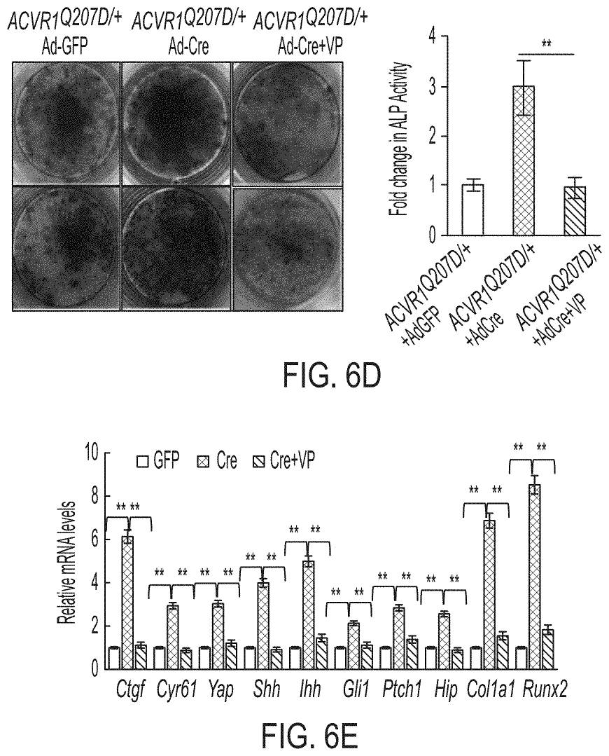

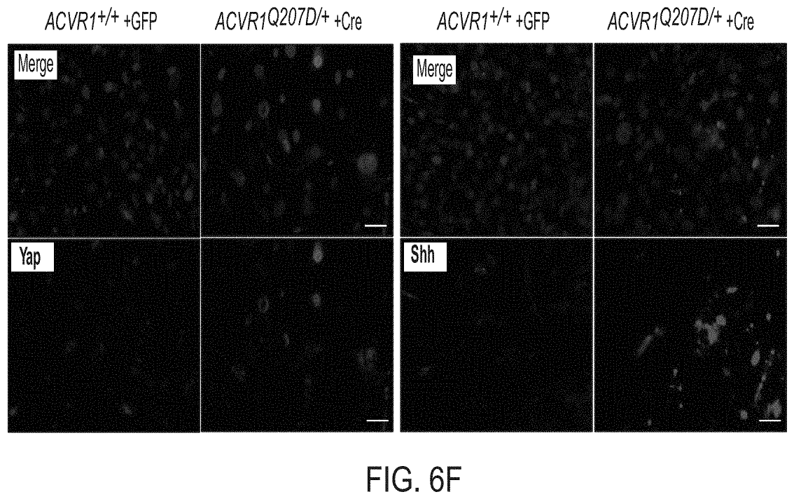

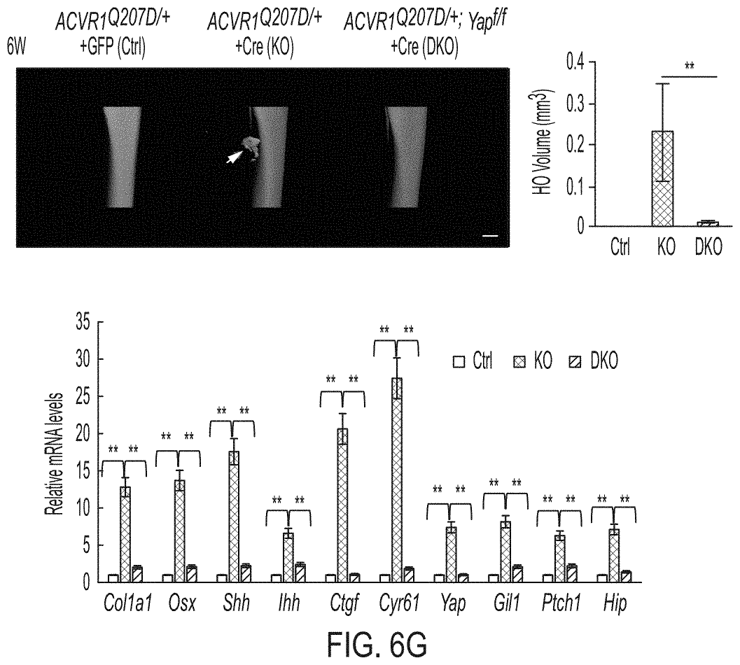

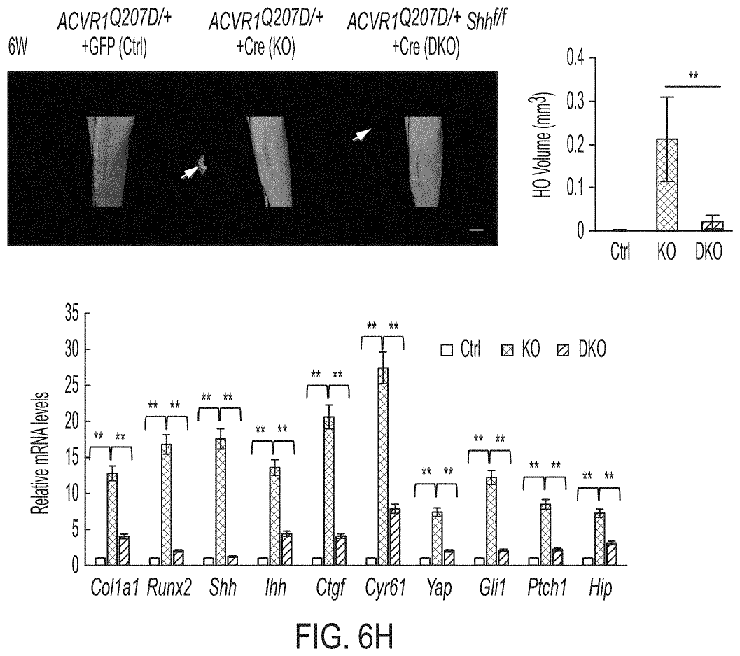

The present invention re-established the ScxCre; ACVR1^{Q207D/+} HO model (FIG. 6A) and found by QRT-PCR analysis that expression of Yap and Hh target genes, Shh, Ihh, as well as Tgfβ1-3 were upregulated in the mutant Achilles tendon (FIG. 6B). Ectopic expression of Yap, Shh, Osx and Opn was detected by immunofluorescent staining in the mutant Achilles tendon (FIG. 6C). Interestingly, while many cells with higher Yap or Shh expression also expressed osteoblast markers Opn and Osx, many Yap⁺; Opn⁻ or Shh⁺; Osx⁻ cells were also detected, suggesting that these cells may have been primed to be differentiated into osteoblasts. In addition, in the ACVR1^{Q207D/+} SMPs, Pdgfrα was also expressed (FIG. 9D) and Pdgfrα+ fibro/adipogenic cells contribute to HO in FOP models. Indeed, osteoblast differentiation was enhanced and expression of Yap and Hh target genes, Shh and Ihh were increased, all of which were reduced by Yap inhibition with VP treatment (FIGS. 6D and 6E). In addition, Ad-Cre infection increased Yap⁺ and Shh⁺ cells in ACVR1^{Q207D/+} SMPs (FIG. 6F). These results suggest that Yap and Shh and/or Ihh are also required for HO in FOP models.

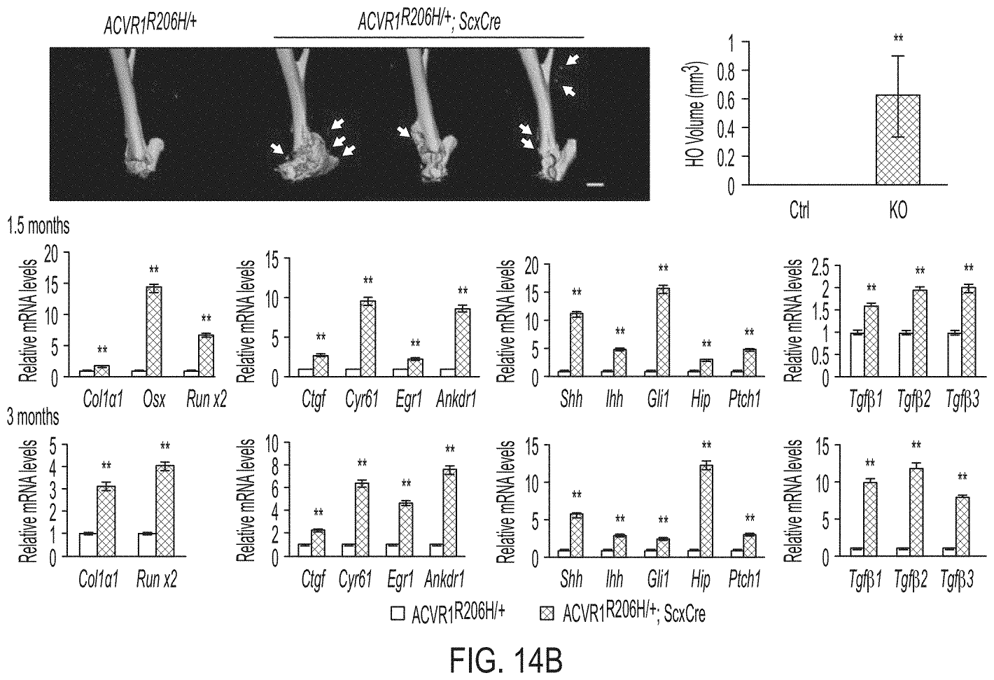

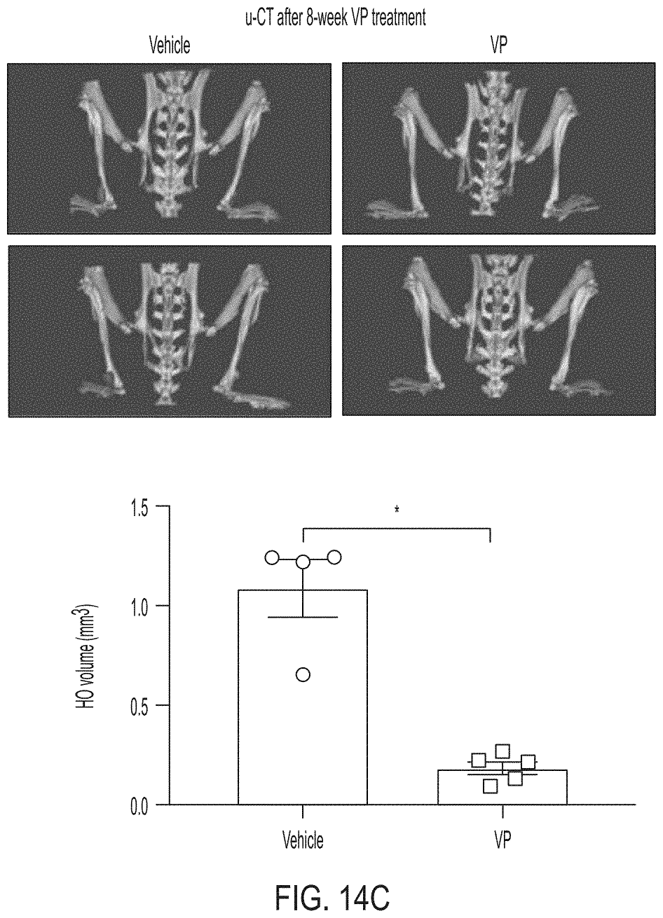

To determine whether Yap and Shh are also required for HO in FOP, the Yap^{f/f} ACVR1^{Q207D/+} mice were generated and Yap was removed while ACVR1^{Q207D/+} expression was activated upon injection of Ad-Cre and cobra venom factor (Wang, X., et al. Nature communications 9, 551 (2018)). Heterotopic bone was detected 6 weeks after injection and was completely abolished by Yap deletion (FIG. 6G). Consistently, expression of osteoblast markers, Yap and Shh signaling target genes as well as Shh/Ihh were significantly reduced (FIG. 6G). Similarly, when Shh is removed in the Shh^{f/f}; ACVR1^{Q207D/+} mice with the same approach, HO as well as the expression of osteoblast markers, Yap and Hh target genes as well as Ihh were significantly decreased (FIG. 6H). In addition, in the ScxCre; ACVR1^{Q207D/+} FOP model, reduction of Yap in the ScxCre; ACVR1^{Q207D/+}; Yap^{f/+} mice led to drastically reduced HO (FIG. 14A). Consistently, expression of osteoblast markers, Shh/Ihh, Hh and Yap signaling targets were also drastically reduced (FIG. 14A). These results show that Yap and Shh upregulation are also required for HO in FOP mouse models. Furthermore, as ACVR^{R206H} was found in human FOP patients, it was investigated whether Yap inhibition could reduce HO in the ScxCre; ACVR^{R206H} FOP model (Hatsell, S. J., et al. Sci Transl Med 7, 303ra137 (2015)). HO was detected in the tendon in 3 months old ACVR1^{R206H}; ScxCre mice and expression of Yap target genes, Shh, Hh target genes, and Tgfβ1-3 were increased in both 1.5 months and 3 months old ACVR1^{R206H}; ScxCre mice (FIG. 14B). Treatment of the ACVR1^{R206H}; ScxCre mice with VP (4 mg/kg/d, 5 days per week) starting at 6 weeks of age for 8 weeks, significantly reduced tendon, ligament, and intra-articular HO (FIG. 14C). The results suggested that VP treatment indeed significantly reduced HO in the ACVR^{R206H} FOP model (FIGS. 14B and 14C).

### Example 7. Yap and Shh are Upregulated in and Required for HO in an Injury-Induced HO Mouse Model

As Yap and Shh are required for HO in both POH and FOP mouse models that are controlled by the two ossification mechanisms, it was further tested whether they are required for acquired HO as genetic models provide critical insights into more common non-genetic conditions. A trauma-induced HO mouse model of percutaneous Achilles tendon puncture (ATP) was used (Wang, X., et al. Nature communications 9, 551 (2018)).

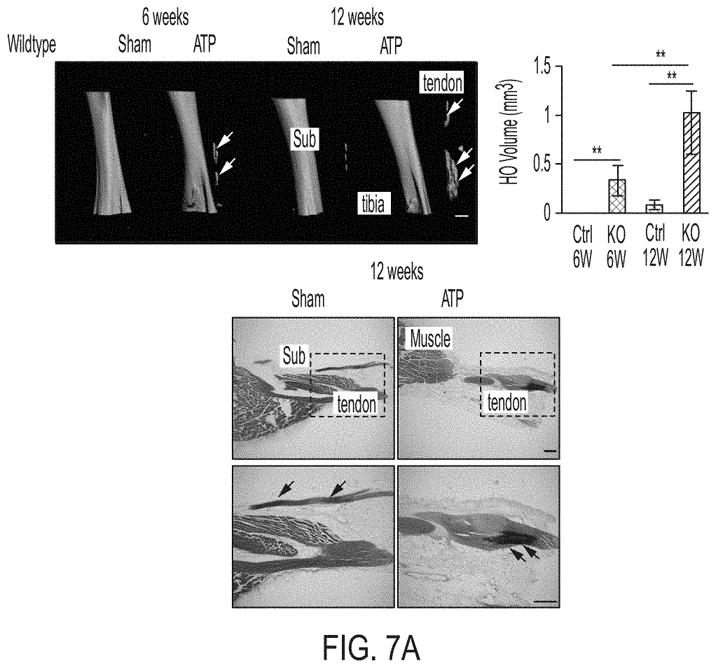

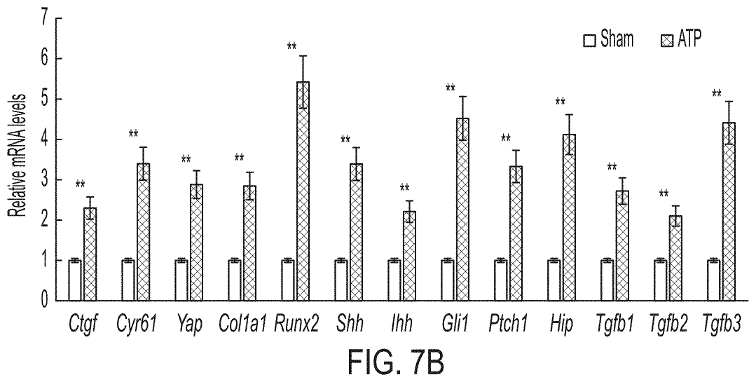

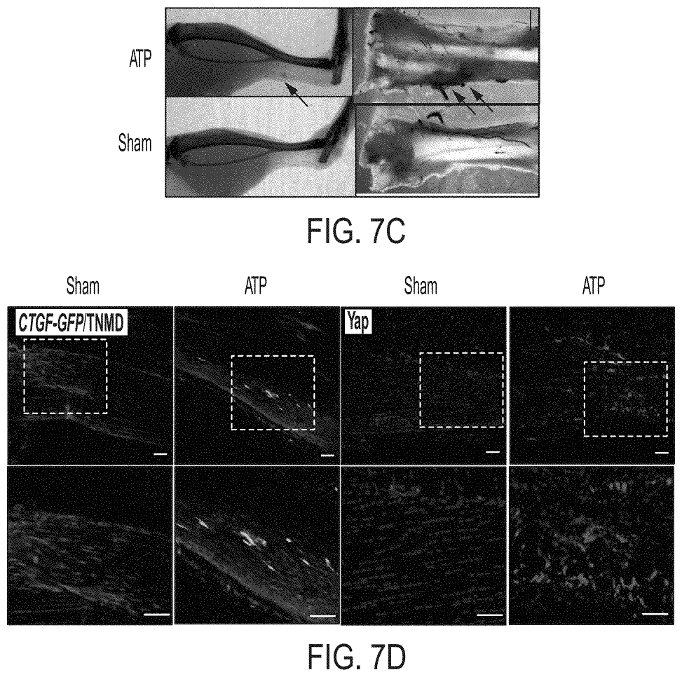

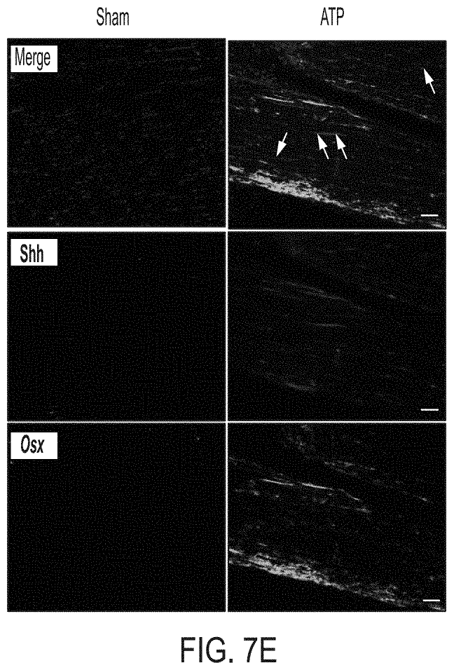

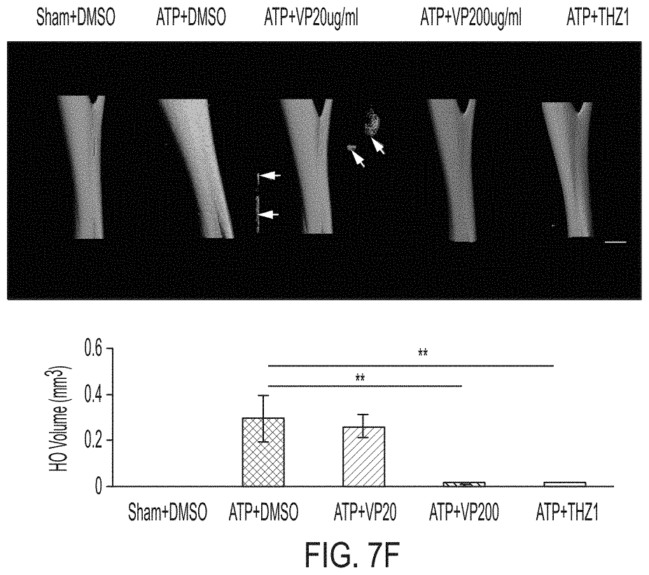

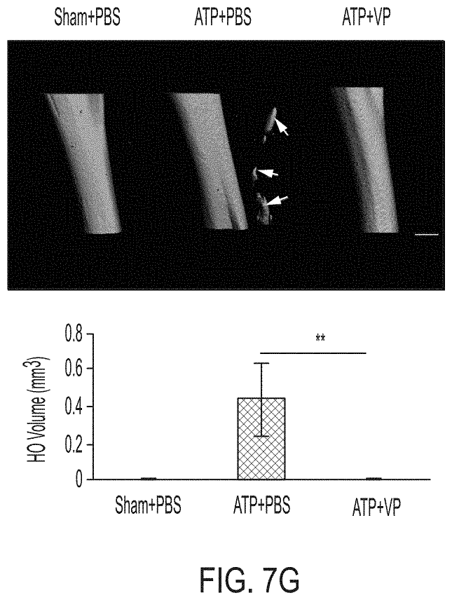

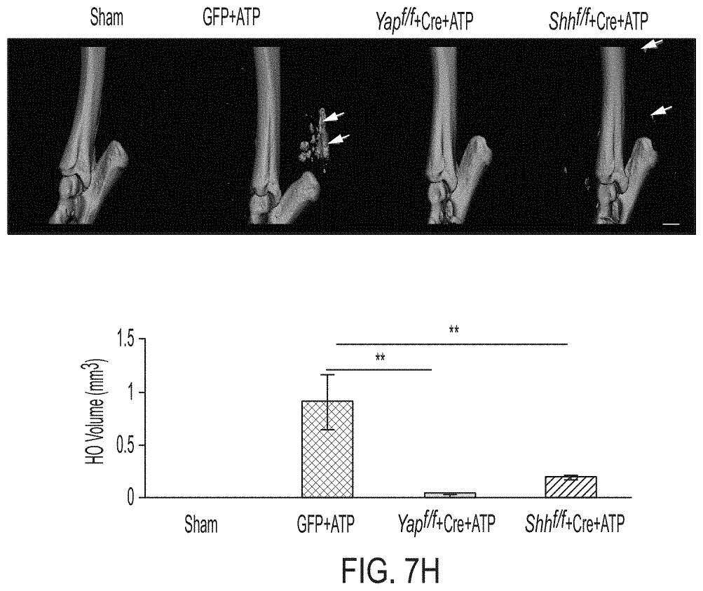

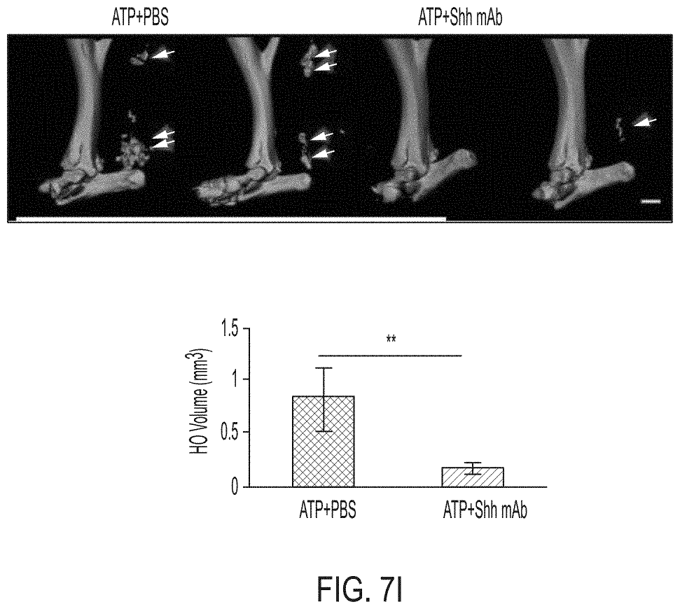

Heterotopic bone was formed in the trauma area of the ATP mouse model 6 weeks post puncture and continued to enlarge up to 12 weeks (FIG. 7A). Weaker mineralization in the subcutaneous area in the sham group was also observed. Expression of Col1a1, Runx2, Yap and Hh signaling target genes, Shh as well as Tgfβ1-3 were increased in the injured Achilles tendon based on QRT-PCR analysis (FIG. 7B). Hh signaling and Yap activities were further examined in vivo in the injured tendon in the Ptch1^{lacZ} and Ctgf-GFP mice, respectively, in which LacZ expression is a readout of Hh signaling activity and GFP expression is controlled by Yap (Yimlamai, D., et al. Cell 157, 1324-1338 (2014); Goodrich, L. V., et al. Science 277, 1109-1113 (1997)). Strong ectopic β-gal staining and GFP expression were observed 10 days post puncture in the Achilles tendon (FIGS. 7C and 7D). Immunostaining indeed showed increased Shh and Osx expression in the injured Achilles tendon 6 weeks post puncture (FIG. 7E).

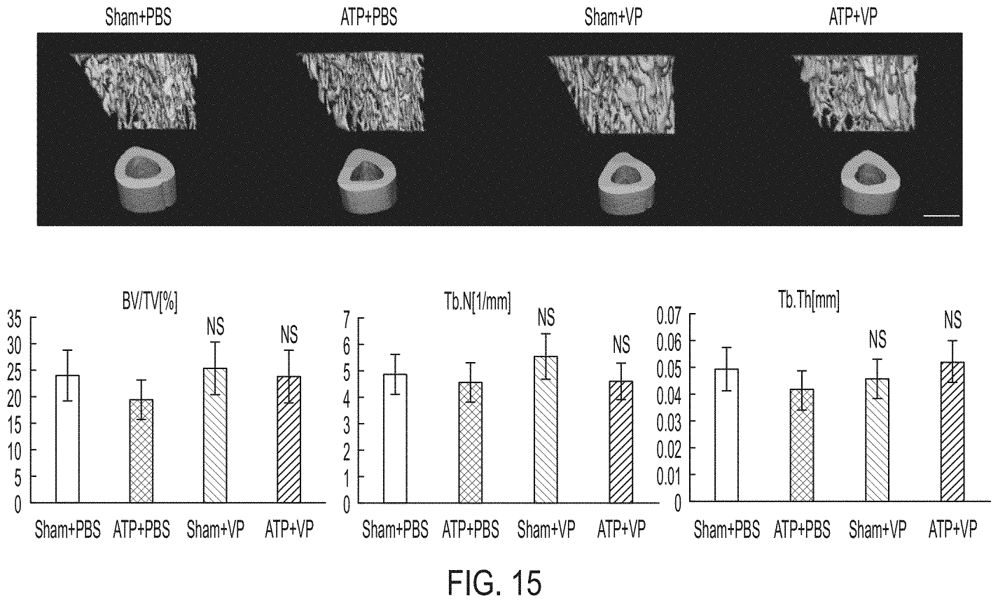

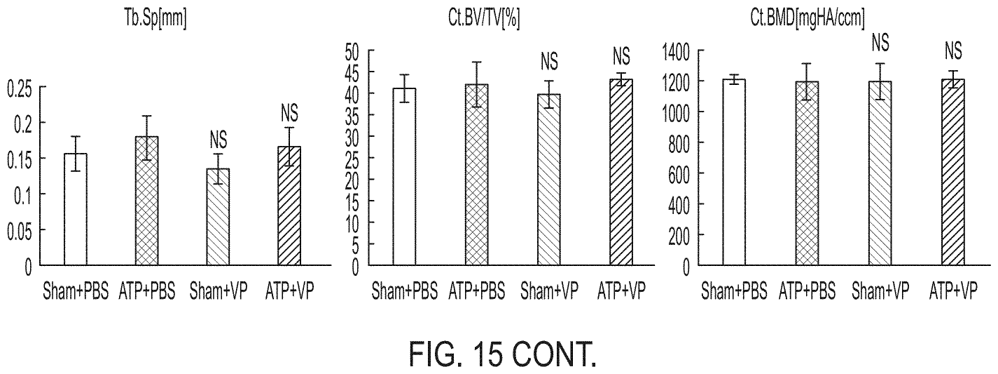

To test whether pharmacological inhibition of Yap attenuates HO in the ATP model, the Yap inhibitor VP and CDK7 inhibitor THZ1 were applied on the skin of the trama area three times a week from the day of ATP for 6 weeks. HO formation was reduced by low dose of VP (20 μg/ml) treatment, but completely blocked by higher doses of VP (200 μg/ml) and THZ1 compared to vehicle treated mice (FIG. 7F). Similar HO blockage was observed in the mice intraperitoneally injected with VP 5 times a week (2.5 mg/kg) from the day of ATP for 6 weeks (FIG. 7G). To determine whether Yap inhibition by VP treatment may reduce endogenous bone mass in ATP mouse model, the tibia bones were analyzed and no significant difference was found between VP treated and control groups (FIG. 15). To further demonstrate the essential role of Yap and Shh in HO, Yap or Shh was removed by injecting Ad-cre to Achilles tendon of the Yap^{f/f} and Shh^{f/f} mice, respectively. Both led to reduced HO caused by injury (FIG. 711). Furthermore, injection of Shh monoclonal antibodies to the WT mice greatly reduced HO caused by ATP (FIG. 7I), suggesting a Shh monoclonal antibody-based therapeutic treatment strategy for HO.

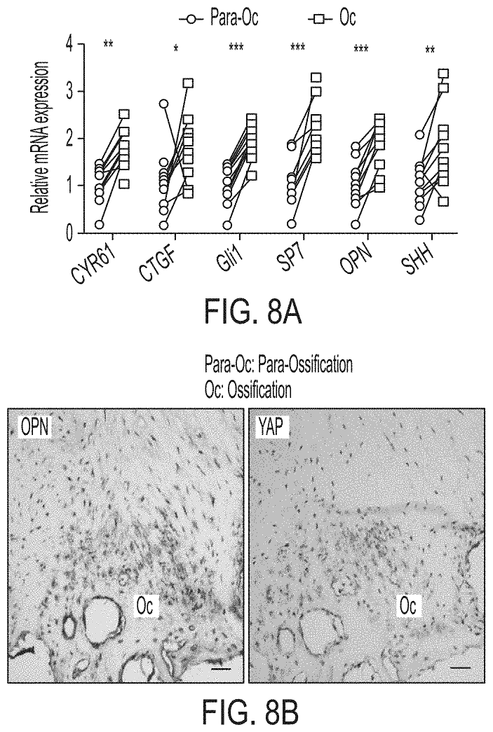

To determine whether upregulation of YAP activity and SHH signaling also contribute to human HO formation, more than 28 human HO samples were collected from Ossification of the Posterior Longitudinal Ligament (OPLL) patients and QRT-PCR analysis and immunostaining of HO and neighboring normal tissues were performed. OPLL is a multi-factorial disease involving HO in spinal ligaments, which causes serious neurological problems and affects 0.8-3.0% aging Asian and 0.1-1.7% aging European Caucasian. In agreement with the data from the mouse models, the results suggested that expression of YAP target genes CTGF and CYR61, SHH, GLI1 and osteoblast markers SP7 (OSX) and OPN were all upregulated in ossified tissues compared to the surrounding soft tissues (FIG. 8A). In addition, immunohistochemistry confirmed that YAP and OPN expression was upregulated in ossified tissues of OPLL patients (FIG. 8B).

Taken all data together, the present invention successfully demonstrated that Yap and Shh activation is a common molecular mechanism whereby HO is induced and expanded in both genetic and acquired HO. Inhibition of HO by Yap inhibitor or Shh inhibitor, e.g., antibody, provide novel therapeutic strategies to treat HO.

### DISCUSSION

The present invention have unexpectedly identified a positive feedback loop between Yap transcription activation and Shh ectopic expression as an essential and fundamentally important core mechanism underlying bone formation and expansion in HO. Importantly, this positive feedback loop and the resulted self-propagation of osteoblast differentiation of wild type mesenchymal progenitor cells during progressive HO (FIG. 12G) are novel shared molecular and cellular mechanisms underlying two distinct genetic HO and injury-induced, common and non-hereditary HO in mice. As YAP and SHH are also upregulated in human HO, findings of this present invention hold promise for new strategies of more effective treatment of human HO, which is still a significantly unmet medical change. In this regard, the present invention shows that new therapeutic approaches based on the mechanistic findings were able to prevent HO formation, block and even shrink expansion of already formed HO in mouse models. The present invention highlights the importance of rare disease studies in identifying pathological mechanisms that are also applied to common diseases, and provides a mechanistic answer to a critical clinical problem in HO, the progressive expansion of the ectopic bone that eventually generates severe pain and interferes with normal muscular-skeletal functions.

Yap is a critical transcription factor in the Hippo signaling pathway that regulates a diverse array of fundamental cellular activities including cell proliferation, differentiation and survival (Zheng, Y. & Pan, D. Dev Cell 50, 264-282 (2019); Meng, Z., et al. Genes Dev 30, 1-17 (2016)). Upregulated Yap expression and activity are found in various cancers. Relevant to the present invention, Gαₛ has been found to cell autonomously control YAP activities in basal cell carcinomas and schwann cells and intraductal papillary mucinous neoplasms (IPMNs) (Iglesias-Bartolome, R., et al. Nat Cell Biol 17, 793-803 (2015); Ideno, N., et al. Gastroenterology 155, 1593-1607 e1512 (2018)). In the basal cell carcinomas (BCC) mouse model, loss of Gnas in the epidermal stem cell compartment cell autonomously activated Yap and Gli transcriptional activities (Iglesias-Bartolome, R., et al. Nat Cell Biol 17, 793-803 (2015)). Furthermore, activation of Hh signaling in the epidermal stem cell compartment in BCC is sufficient to activate Yap, loss of which blocked BCC development without dampening Hh or Wnt signaling actitives (Iglesias-Bartolome, R., et al. Nat Cell Biol 17, 793-803 (2015)). Conversely, in the pancreatic ductal adenocarcinomas (PDACs) induced by activated KRAS in mice (Potter, B. K., et al. J Am Acad Orthop Surg 14, S191-197 (2006)), expression of a constitutive active form of Gnas led of Hippo signaling activation and cytoplasmic sequestration of phosphorylated Yap. However, the relationship between Hh signaling and Yap is context-dependent. In the mesenchyme of the developing gut, zone-specific Yap/Taz downregulation enables smooth muscle specification by Hh Activation (Cotton, J. L., et al. Dev Cell 43, 35-47 e34 (2017)).

As abnormal bone growth in soft tissues such as muscle causes pain and interferes with normal musculoskeletal function, it is essential to block ectopic bone formation and expansion in HO treatment or prevention after its surgical removal. In this regard, identification of previously unknown cell non-autonomous role of Yap in activating and propagating Hh signaling activation to surrounding cells by directly activating Shh expression and also responding to Hh signaling represents a major advancement in the cellular and molecular mechanisms underlying abnormal bone growth in disorders with HO. The present invention was made with combined genetic, genomic and biochemical studies of the subcutaneous mesenchymal progenitor cells that can be induced by Hh signaling to become osteoblast cells. Yap has been found to serve as a co-regulator for phosphor-smad1/5/8, Runx2, and β-catenin (Huang, Z., et al. Development 143, 2398-2409 (2016); Zaidi, S. K., et al. EMBO J23, 790-799 (2004); Azzolin, L., et al. Cell 158, 157-170 (2014), all of which promote osteoblast differentiation. However, Yap in mesenchymal progenitor cells is not required for bone formation in development (Xiong, J., Almeida, M. & O'Brien, C. A. Bone 112, 1-9 (2018); Kegelman, C. D., et al. FASEB J 32, 2706-2721 (2018)), consistent with the observation that pharmacological inhibition of Yap did not reduce bone mass of the normal bone. As Yap genetic removal or pharmacological inhibition led to complete loss of HO (FIG. 3B), regulation of ectopic osteoblast differentiation is not the same as that in normal bone development or homeostasis. This notion is further supported by the surprisingly critical function of Shh in HO. Shh was only found previously to be required for early embryonic growth and patterning, but not bone formation. The present invention teaches that Ihh was also upregulated by Gas loss, but not as robustly as Shh (FIGS. 2C and 10A). In the FOP models, because of the presence of cartilage where Ihh is normally expressed and induces osteoblast differentiation (St-Jacques, B., Hammerschmidt, M. & McMahon, A. P. Genes Dev 13, 2072-2086. (1999)), Ihh was expressed at a stronger level, similar to Shh, in the ectopic bone (FIGS. 6B and 14A). In addition, it was found that Wnt signaling is upregulated in Gnas^{-/-} cells. It is possible that Ihh removal or Wnt inhibition could also reduce or block HO. However, YAP and SHE are much better therapeutic targets as they are not required for normal bone formation. Between Yap and Shh, Shh inhibition appeared to be less potent in blocking HO (FIG. 2G). This is likely due to the redundant functions of Ihh and cell autonomous role of Gas in suppressing Gli transcription activities independently of Hh ligands. Loss of Gas should still be able to activate Hh signaling to induce osteoblast differentiation cell-autonomously in the absence of Shh. Therefore, Shh strongly potentiates Hh signaling initially activated by Gas loss, contributing to rapid HO expansion. Furthermore, the present invention shows that Yap is not acting independently of Hh signaling as indicated (Iglesias-Bartolome, R., et al. Nat Cell Biol 17, 793-803 (2015)). Instead, it interacts intimately with Hh signaling by acting both downstream and upstream of Hh signaling activation in SMPs. Abnormal bone expansion and frequent recurrence after surgical removal of HO bear certain similarities to rapid tumor growth and recurrence. Given the significant pro-tumor growth roles of Yap and Hh signaling activation, it is possible that similar regulatory positive feedback loop also exists in tumors, causing rapid tumor growth. The present invention showed YAP-TEAD-AP1 cooperation by directly interacting on the target gene promoters to regulate their expression (FIGS. 5D and 5E), which has already been described in tumors (Zanconato, F., et al. Nat Cell Biol 17, 1218-1227 (2015); Iglesias-Bartolome, R., et al. Nat Cell Blot 17, 793-803 (2015); Liu, B., et al. Cell 164, 406-419 (2016)). In addition, given the critical roles of Shh in embryonic growth and patterning, it is possible that Yap is required to regulate Shh expression in a particular developmental process.

The present invention highlights the invaluable contributions of rare disease studies in identifying the core cellular and molecular processes that are also affected in non-inheritable common diseases. The Yap-Shh positive feedback loop in ectopic bone formation and growth was first found in POH mouse models, but also found to operate in several FOP mouse models in which ectopic bone was induced by constitutively active ALK2/ACVR1 mutants. As POH and FOP are two genetic diseases with HO arisen by two distinct ossification mechanisms, the present invention also successfully demonstrated that both Yap and Shh are essential for injury induced acquired HO and furthermore, human non-hereditary HO samples exhibit increased YAP and SHH expression. In mouse models of POH, FOP and ATP, genetic and pharmacological inhibition of Yap and Shh effectively blocked ectopic bone formation and expansion in HO, suggesting that inhibition of YAP and SHH may also inhibit HO in human patients. These findings provide a strong base for strategic development of more effective HO treatment.

### EQUIVALENTS

Those skilled in the art will recognize, or be able to ascertain using no more than routine experimentation, many equivalents to the specific embodiments of the invention described herein. such equivalents are intended to be encompassed by the following claims. The contents of all references, patents and published patent applications cited throughout this application are incorporated herein by reference.

1. A method of inhibiting heterotopic ossification (HO) of a cell, comprising
contacting the cell with an agent that inhibits the expression and/or the activity of Yes-associated protein (Yap),
thereby inhibiting heterotopic ossification of the cell.

2. The method of claim 1, wherein the activity of Yap is transcriptional activation of sonic hedgehog (Shh) expression and/or enhancing Shh protein activity.

3. The method of claim 2, wherein the Shh protein activity is enhancing Yap expression.

4. The method of claim 1, wherein the cell is a mesenchymal progenitor cell, a mesenchymal stem cell, or a mesenchyme derived differentiated cell.

5. The method of claim 1, wherein the contacting occurs in vitro.

6. The method of claim 1, wherein the cell is within a subject.

7. The method of claim 6, wherein the subject is a human subject.

8. The method of claim 7, wherein the subject has heterotopic ossification (HO).

9.-12. (canceled)

13. The method of claim 7, wherein the subject is at risk of developing heterotopic ossification (HO).

14. (canceled)

15. The method of claim 1, wherein the agent that inhibits the expression and/or activity of Yap is selected from the group consisting of a small molecule, an anti-Yap antibody-or antigen-binding fragment thereof, an antisense agent targeting Yap, a double stranded RNA agent targeting Yap, an RNA-guided nuclease targeting Yap, a Yap fusion protein; a Yap inhibitory peptide; an anti-Shh antibody-or antigen-binding fragment thereof, an antisense agent targeting Shh, a double stranded RNA agent targeting Shh, an RNA-guided nuclease targeting Shh, an Shh fusion protein; an Shh inhibitory peptide, and a CDK7 inhibitor

16. The method of claim 15, wherein the agent is Verteporfin (VP).

17. The method of claim 15, wherein the CDK7 inhibitor is THZ1 or CT7001.

18. The method of claim 15, further comprising inhibiting the expression level and/or activity of hedgehog (Hg) signaling pathway; further comprising inhibiting the expression level and/or activity of Shh; or further comprising inhibiting the expression level and/or activity of CDK7.

19. (canceled)

20. (canceled)

21. A method of treating a subject having heterotopic ossification (HO) or a subject at risk of developing HO, comprising
administering to the subject a therapeutically effective amount of an inhibitor of Yes-associated protein (Yap) expression and/or the activity,
thereby treating the subject.

22.-26. (canceled)

27. The method of claim 21, wherein the agent that inhibits the expression and/or activity of Yap is selected from the group consisting of a small molecule, an anti-Yap antibody-or antigen-binding fragment thereof, an antisense agent targeting Yap, a double stranded RNA agent targeting Yap, an RNA-guided nuclease targeting Yap, a Yap fusion protein; a Yap inhibitory peptide; an anti-Shh antibody-or antigen-binding fragment thereof, an antisense agent targeting Shh, a double stranded RNA agent targeting Shh, an RNA-guided nuclease targeting Shh, an Shh fusion protein; an Shh inhibitory peptide, and a CDK7 inhibitor.

28. The method of claim 27, wherein the agent is Verteporfin (VP).

29. The method of claim 27, wherein the CDK7 inhibitor is THZ1 or CT7001.

30. A method for identifying a compound that inhibits heterotopic ossification (HO) of a cell, comprising providing a cellular indicator composition, contacting the indicator composition with a test compound, determining the effect of the compound on the expression and or activity of YAP in the indicator composition, wherein a decrease in the expression and/or activity of Yap indicates that the test compound inhibits HO, thereby identifying a compound that inhibits HO of the cell.

31. The method of claim 30, wherein the indicator composition comprises a mesenchymal progenitor cell, a mesenchymal stem cell, or a mesenchyme derived differentiated cell.

32. The method of claim 30, wherein the indicator composition comprises a cell comprising an inactive Gnas gene; a cell comprising a constitutively active caALK2 gene; or a cell comprising an ACVR^{R206H} gene.

33.-36. (canceled)
